(19) United States
(12) Patent Application Publication
(10) Pub. No.: US 2024/0173483 A1
(43) Pub. Date: May 30, 2024

(54) DEVICES AND METHODS FOR DELIVERY OF SUBSTANCES WITHIN A PREFILLED SYRINGE

(71) Applicant: kaleo, Inc., Richmond, VA (US)

(72) Inventors: Paul F. MEYERS, Fishers, IN (US); Michael J. ROE, Powhatan, VA (US)

(73) Assignee: kaleo, Inc., Richmond, VA (US)

(21) Appl. No.: 18/385,618

(22) Filed: Oct. 31, 2023

Related U.S. Application Data
(60) Provisional application No. 63/428,557, filed on Nov. 29, 2022.

(51) Int. Cl. A61M 5/20; A61M 39/22

(52) U.S. Cl. CPC A61M 5/2046; A61M 39/22

(57) ABSTRACT

An apparatus includes a housing, a gas container, a medicament container assembly, and a movable seal member, with the housing defining a gas chamber and an equalization bypass. The gas container is disposed within the housing and configured to produce pressurized gas within the gas chamber when the apparatus is actuated. The movable seal member is configured to move from a first seal position to a second seal position. When the movable seal member is at the first seal position, the gas chamber is in fluid communication with the exterior volume surrounding the apparatus via the equalization bypass. When the movable seal member is at the second seal position, the gas chamber is fluidically isolated from the exterior volume by the movable seal member.

### CROSS-REFERENCE TO RELATED APPLICATIONS

This application claims benefit of priority to U.S. Provisional Application No. 63/428,557, entitled “Devices and Methods for Delivery of Substances Within a Prefilled Syringe,” filed Nov. 29, 2022, which is incorporated herein by reference in its entirety.

### BACKGROUND

The embodiments described herein relate to medicament delivery devices, pharmaceutical compositions, and drug products. More particularly, the embodiments described herein relate to medicament delivery devices for delivery of medicaments contained within a prefilled syringe.

Known prefilled syringes are commonly used to contain and inject medicaments. Known prefilled syringes include a syringe body, often constructed from glass, within which a medicament is contained. The distal end portion of some known prefilled syringes includes a staked needle (i.e., a needle that is permanently coupled to the syringe body during manufacture), the end of which is disposed within a needle cover to maintain the sterility of the needle prior to use. Other known prefilled syringes include a Luer fitting or adapted such that the distal end portion of the syringe body can be coupled to a needle. The proximal end portion of the syringe body of known prefilled syringes includes a plunger (usually constructed from an elastomer) that defines a portion of the container closure, and that can be moved within the syringe body to inject the medicament. The proximal end portion also includes a flange to allow the user to grasp the syringe body and manually apply a force to a piston to move the plunger, thereby causing injection of the medicament.

Although prefilled syringes can be cost effective devices for storing and delivering medicaments, known methods for using prefilled syringes include manually inserting the needle into the body followed by manually applying the injection force. Moreover, upon completion of the injection, known methods include covering the needle to avoid needle sticks. Thus, known prefilled syringes are often used by healthcare professionals that are trained in such procedures. To facilitate the self-administration of medicaments contained in prefilled syringes, some known autoinjectors have been adapted to contain prefilled syringes. Such known devices include a source of stored energy for inserting the needle and/or injecting the medicament.

Known autoinjectors, however, are often designed for a medicament container having a specific size and/or shape, and are therefore often not configured to receive known prefilled syringes. For example, using a prefilled syringe within a known autoinjector can often result in high forces being applied to the flange of the syringe body during the insertion operation, which can lead to breakage of the syringe flange or body. Moreover, because many known prefilled syringes include a staked needle that is in fluid communication with the medicament, applying a force to the plunger during storage and/or during an insertion operation is undesirable. For example, the application of a force against the plunger during storage, which can result, for example, when a spring-loaded member is placed in contact with the plunger, can cause in leakage of the medicament. As another example, the application of a force against the plunger during a needle insertion event can result in the injection of the medicament before the needle is inserted to the desired location. Similarly stated, some known auto-injectors are not configured to control the force applied to the plunger within the syringe body during storage and/or needle insertion.

Known autoinjectors configured to incorporate a prefilled syringe often include a spring-based actuation system that moves a piston rod to insert the needle and inject the medicament. The size (e.g., length) of such known systems, however, can be larger than desired because of the need to incorporate the piston rod. Moreover, known medicaments or therapeutic substances are formulated to include high molecular weight compounds, compounds with complex molecular structures, living cells, and/or biologics. Such medicaments often have a very high viscosity (e.g., greater than about 100 centipoise at room temperature), which must be accommodated by the delivery system. For example, the force and pressure necessary to overcome the resistance of a spring-based actuation system in an autoinjector may be incompatible with the force and pressure required for the proper delivery of medicaments or therapeutic substances including high molecular weight compounds. Accordingly, many known auto-injectors that accommodate a prefilled syringe may not be able to provide appropriate force and/or develop the desired flow rate for effective delivery of such higher viscosity substances. Moreover, even if an auto-injector is capable of producing the desired force, such devices may result in undesirable delivery conditions or rates, which can compromise the substance being delivered or cause excessive pain or discomfort during the delivery process. For example, if the rate of delivery is too high, the resulting shear forces may damage the molecules within the substance, thereby reducing efficacy. Furthermore, known auto-injectors that include automatic needle retraction after delivery of the medicament to prevent accidental needle prick often include one or more guide shafts or linkages to detect the completion of the medicament delivery in order to initiate the retraction process. The guide shafts and linkages introduce additional friction during the medicament delivery process that works against providing desirable delivery rates and conditions. The guide shafts and linkages also require additional internal housing space, thereby increasing the overall size and bulk of the auto-injector.

To address the challenges associated with spring-based actuation system, some known systems use a pressurized gas to generate the required insertion and/or medicament delivery forces. Typically, such systems utilize a gas chamber that receives a pressurized gas from a gas container when the system is actuated. The gas chamber is typically sealed at time of manufacture and has an internal pressure that corresponds to the atmospheric pressure of the manufacturing location at time of manufacture. Accordingly, in such known systems, a pressure differential can develop between the pressure within the gas chamber and the atmospheric pressure surrounding the device due to changes in the pressure and/or temperature of the environment in which the device is located. The pressure differential can negatively affect the device in a stored state and/or negatively impact the functioning of the device upon actuation. For example, when the device is exposed to altitudes that are higher than the place of manufacture (e.g., during shipment by air), the pressure surrounding the device may be less than the pressure within the gas chamber. In such an instance, the greater pressure within the gas chamber can result in leakage of the medicament. Similarly, when the device is exposed to altitudes that are lower than the place manufacture, the pressure surrounding the device may be greater than the pressure within the gas chamber. In such an instance, the negative pressure differential can preclude complete delivery of the medicament upon actuation of the device.

Thus, a need exists for improved methods and devices for delivering medicaments contained within a prefilled syringe.

### SUMMARY

Medicament delivery devices for administration of medicaments contained within a prefilled syringe are described herein. In some embodiments, an apparatus includes a housing, a gas container, a medicament container assembly, and a movable seal member. The housing defines a gas chamber and an equalization bypass. The gas container is configured to produce a pressurized gas within the gas chamber when the apparatus is actuated. The medicament container assembly is positioned within the housing and includes a container body and an elastomeric member. The elastomeric member is positioned within the container body and configured to move within the container body to convey a medicament contained therein. The elastomeric member is configured to move within the container body in response to a force exerted by the pressurized gas within the gas chamber. The movable seal member is also positioned within the housing. The movable seal member is configured to move from a first seal position to a second seal position. When the movable seal member is at the first the scal position, the gas chamber is in fluid communication with the exterior volume surrounding the housing via the equalization bypass. However, when the movable seal member is at the second position, the gas chamber is fluidly isolated from the exterior volume by the movable seal member.

In some embodiments, the apparatus includes a system actuator assembly that has a release member and a puncturer coupled to the release member. The release member is movable within the housing between a first release member position and a second release member position. The puncturer is spaced apart from the gas container when the release member is in the first release member position. The puncturer pierces a portion of the gas container when the release member is in the second release member position. The movable seal is coupled to the release member. The movable seal being at the first seal position when the release member is in the first release member position. The movable seal is in the second seal position when the release member is at the second release member position.

In some embodiments, the apparatus includes a system actuator assembly that has a release member, a puncturer, and an actuation spring disposed within the housing. The release member is movable within the housing. The release member is in a locked configuration and the actuation spring has a stored-energy configuration prior to actuation of the apparatus. The release member is transition to a released configuration in response to an actuation force being exerted by the actuation spring on the release member when the apparatus is actuated. The puncturer is coupled to the release member and positioned to puncture the gas container in response to the release member transitioning from the locked configuration towards the released configuration. The movable seal is coupled to the release member and is positioned between the release member and an inner wall of the housing. The movable seal is at the first seal position when the release member is in the locked configuration, and the movable seal is configured to move to the second seal position in response to the release member transitioning from the locked configuration towards the released configuration.

In some embodiments, the system actuator assembly includes a positioning member coupled to the release member. The positioning member has a first shape when the release member is in the locked configuration and a second shape when the release member is in the released configuration. The positioning member engages a receiving portion of the housing when in the second shape. The movable seal is maintained at a third seal position when the positioning member engages the receiving portion of the housing. The gas chamber is fluidically isolated from the exterior volume by the movable seal member when the movable seal member is at the third seal position. In some embodiments, the third seal position is between the first seal position and the second seal position along a path of motion.

In some embodiments, the apparatus includes a carrier coupled to the medicament container assembly and configured to move within the housing in response to the force exerted by the pressurized gas. A proximal surface of the carrier defines a portion of a boundary of the gas chamber.

In some embodiments, the movable seal is coupled to the carrier and is positioned between the carrier and an inner wall of the housing. The movement of the carrier in response to the force exerted by the pressurized gas causes the movable seal to move relative to the housing from the first seal position to the second seal position to isolate the gas chamber from the exterior volume.

In some embodiments, the apparatus includes a gas vent assembly configured to selectively place the gas chamber in fluid communication with the exterior volume. The gas vent assembly includes a valve member configured to seal a vent opening defined by the housing. The valve member occludes the vent opening when at a first valve position prior to actuation of the apparatus, and the valve member is configured to move within the housing from the first valve position to a second valve position in response to the movement of the elastomeric member. The gas chamber is in fluid communication with the exterior volume via the vent opening when the valve member is at the second valve position.

In some embodiments, the apparatus includes an expandable assembly that has a first member, a second member and a third member. The first member is coupled to the elastomeric member, the second member is coupled between the first member and the third member, and the third member is coupled to the valve member. The expandable assembly is configured to transition from a first configuration to a second configuration when the elastomeric member moves within the container body. The valve member moves from the first valve position to the second valve position when the expandable assembly transitions from the first configuration to the second configuration to release the pressurized gas from the gas chamber to the exterior volume.

In some embodiments, the vent opening is sized to maintain the force of the pressurized gas within the gas chamber at a magnitude that is greater than a force applied by a retraction spring during a time to dispense the medicament, the force of the pressurized gas decreasing to a magnitude that is less than a force applied by the retraction spring concurrent with a completion of the dispensing of the medicament.

In some embodiments, the vent opening is sized such that the pressurized gas causes a first 20% of the medicament to dispense over a first time duration and a last 20% of the medicament to dispense over a second time duration that is less than two times the first time duration.

In some embodiments, the movable seal member is a first movable seal member. The apparatus includes a carrier coupled to the medicament container assembly and configured to move within the housing in response to the force exerted by the pressurized gas. A proximal surface of the carrier defines a first portion of a boundary of the gas chamber. A second movable seal member is coupled to the carrier and positioned between the carrier and an inner wall of the housing. The second movable seal member defines a second portion of the boundary of the gas chamber.

In some embodiments, the movable seal is configured to move from the first position to the second position in response to a force exerted by the pressurized gas within the gas chamber. The movable seal is configured to move from the second seal position to a third seal position in response to the movement of the elastomeric member, with the gas chamber being in fluid communication with the exterior volume via the equalization bypass when the movable seal member is at the third seal position.

In some embodiments, the apparatus includes a gas vent assembly configured to selectively place the gas chamber in fluid communication with the exterior volume. The gas vent assembly includes a valve member configured as the movable seal and a vent opening defined by the housing. The valve member is configured to seal the vent opening. The gas chamber is in fluid communication with the vent opening via the equalization bypass when the valve member is at the first seal position.

In some embodiments, the apparatus includes an expandable assembly. The expandable assembly has a first member, a second member and a third member. The first member is coupled to the elastomeric member, the second member is coupled between the first member and the third member, and the third member is coupled to the valve member. The expandable assembly is configured to transition from a first configuration to a second configuration when the elastomeric member moves within the container body. The valve member moves from the second seal position to the third seal position when the expandable assembly transitions from the first configuration to the second configuration to release the pressurized gas from the gas chamber to the exterior volume.

In some embodiments, the container body is movable within the housing from a first container position to a second container position in response to the force exerted by the pressurized gas. The movable seal member is coupled to the container body and is positioned between the container body and an inner wall of the housing. The movement of the container body in response to the force exerted by the pressurized gas moves the movable seal relative to the housing from the first seal position to the second seal position to isolate the gas chamber from the exterior volume.

In some embodiments, the apparatus includes a delivery control mechanism coupled to the medicament container assembly. The delivery control mechanism includes a flow restriction member configured to regulate flow of the pressurized gas into the container body that acts on the elastomeric member. The medicament container assembly is configured to move from a first container position to a second container position in response to the force exerted by the pressurized gas, and the flow restriction member is configured to limit movement of the elastomeric member prior to the medicament container assembly being placed in the second container position.

In some embodiments, the gas chamber is a first gas chamber. In such embodiments, the flow restriction member is configured to permit pressurized gas to pass from the first gas chamber to a second gas chamber, which is in fluid contact with the elastomeric member. A first portion of the pressurized gas in the first gas chamber has a first pressure magnitude, and a second portion of the pressurized gas in the second gas chamber has a second pressure magnitude. The first pressure magnitude is greater than the second pressure magnitude.

In some embodiments, the apparatus includes a needle coupled to a distal end portion of the container body.

### BRIEF DESCRIPTION OF THE DRAWINGS

FIGS. 1A and 1B are schematic illustrations of a medicament delivery device according to an embodiment depicting a gas chamber in fluid communication with the exterior volume surrounding the delivery device (FIG. 1A) and the gas chamber being fluidically isolated from the exterior volume (FIG. 1B).

FIG. 2 is a front perspective view of a medical injector according to an embodiment, in a first configuration.

FIGS. 3 and 4 are front and rear perspective views, respectively, of the medical injector illustrated in FIG. 2, with the electronic circuit system hidden and the safety lock removed.

FIG. 5 is a perspective view of a housing of the medical injector illustrated in FIG. 2.

FIG. 6 is a cross-sectional view of the housing illustrated in FIG. 5.

FIGS. 7 and 8 are a perspective view and a cross-sectional view, respectively, of a proximal cap of the medical injector illustrated in FIG. 2.

FIGS. 9 and 10 are front views of a medicament delivery mechanism of the medical injector shown in FIG. 2.

FIG. 11 is a front view of the medical injector shown in FIG. 2, in the first configuration.

FIG. 12 is a front cross-sectional view of the medical injector shown in FIG. 2, in the first configuration.

FIG. 13A is an enlarged cross-sectional view of a portion of the medical injector shown in FIG. 12, in the first configuration.

FIG. 13B is an enlarged cross-sectional view of a portion of the medical injector shown in FIG. 12, in the first configuration.

FIGS. 14A and 14B are a perspective view and a cross-sectional view of a delivery control mechanism of the medical injector shown in FIG. 12.

FIG. 15 is a cross-sectional view of the delivery control mechanism shown in FIGS. 14A and 14B within the proximal end portion of the medicament container.

FIGS. 16 and 17 are a perspective view and a cross-sectional view, respectively, of a carrier assembly of the medical injector shown in FIG. 12.

FIG. 18 is a perspective view of the carrier assembly of the medical injector shown in FIG. 12.

FIG. 19 is a cross-sectional view of the carrier assembly and a medicament container of the medical injector shown in FIG. 12.

FIG. 20 is an exploded view of a medicament container assembly of the medical injector shown in FIG. 12.

FIG. 21 is a perspective view of a gas vent assembly of the medical injector shown in FIG. 12.

FIGS. 22A, 22B and 22C are cross-sectional views of the gas vent assembly of FIG. 21, in a first configuration, a second configuration, and a third configuration, respectively.

FIGS. 23 and 24 are perspective views of a safety lock of the medical injector shown in FIG. 2, with FIG. 23 showing the needle sheath assembly coupled to the safety lock.

FIGS. 25 and 26 are perspective views of a system actuator of the medical injector shown in FIG. 2.

FIG. 27 is a front cross-sectional view of the medical injector shown in FIG. 2, in a second configuration (safety lock removed).

FIG. 28 is a front cross-sectional view of the medical injector shown in FIG. 2, in a third configuration (actuated).

FIG. 29 is a front view of the medical injector shown in FIG. 2, in a fourth configuration (needle inserted).

FIG. 30 is a front cross-sectional view of the medical injector shown in FIG. 2, in the fourth configuration (needle inserted).

FIG. 31 is an enlarged cross-sectional view of the medical injector shown in FIG. 2, in the fourth configuration (needle inserted).

FIG. 32 is a front view of the medical injector shown in FIG. 2, in a fifth configuration (medicament delivered).

FIG. 33 is a front cross-sectional view of the medical injector shown in FIG. 2, in the fifth configuration (medicament delivered).

FIG. 34 is a perspective cross-sectional view of the medical injector shown in FIG. 2, in the fifth configuration (medicament delivered).

FIG. 35 is a front view of the medical injector shown in FIG. 2, in a sixth configuration (housing gas chamber vented).

FIG. 36 is a front cross-sectional view of the medical injector shown in FIG. 2, in the sixth configuration (housing gas chamber vented).

FIG. 37 is a perspective cross-sectional view of the medical injector shown in FIG. 2, in the sixth configuration (housing gas chamber vented).

FIG. 38 is a front view of the medical injector shown in FIG. 2, in a seventh configuration (needle retracted).

FIG. 39 is a front cross-sectional view of the medical injector shown in FIG. 2, in a seventh configuration (needle retracted).

FIG. 40 is a perspective cross-sectional view of the medical injector shown in FIG. 2, in the seventh configuration (needle retracted).

FIG. 41 is a front cross-sectional view of a portion of a medical injector according to an embodiment, with a release member of a system actuator assembly at a first release member position, a movable seal at a first seal position, and a positioning member having a first shape.

FIG. 42 is a front cross-sectional view of the portion of a medical injector according of FIG. 41, with the release member at a second release member position, the movable seal at a second seal position, and the positioning member having a second shape.

FIG. 43 is a front cross-sectional view of the portion of a medical injector according of FIG. 41, with the movable seal at a third seal position, and the positioning member in the second shape.

FIG. 44 is a front cross-sectional view of a portion of a medical injector according to an embodiment, with a movable seal at a first seal position.

FIG. 45 is a front cross-sectional view of a portion of a medical injector according to an embodiment, with a movable seal at a first seal position.

### DETAILED DESCRIPTION

Medicament delivery devices for administration of medicaments contained within a prefilled syringe are described herein. As described, the medicament delivery device (apparatus) uses a pressurized gas in a gas chamber to dispense a medicament contained within a medicament container assembly. The pressurized gas is introduced to the gas chamber from a gas container upon actuation of the apparatus. Prior to actuation, the gas chamber is in fluid communication with the exterior volume that surrounds the apparatus via an equalization bypass. In other words, prior to actuation, the pressure within the gas chamber corresponds to the environmental pressure affecting the apparatus. The apparatus includes a movable seal member. The movable seal member is at a first seal position prior to actuation, and the gas chamber is in fluid communication with the exterior volume via the equalization bypass. However, upon actuation of the apparatus, the movable seal member moves to a second scal position at which the movable seal member isolates the gas chamber from the exterior volume. Said another way, the gas chamber is sealed when the movable seal is at the second seal position.

In some embodiments, the movable seal is one of a set of multiple seal members that collectively form portions of the boundary of the gas chamber and are configured to move in conjunction with various operations of the apparatus. For example, in some embodiments, the apparatus can include a first seal that is coupled to a release member positioned to release the pressurized gas from the gas container. A second seal can be coupled to a carrier of the medicament container or to the container itself. A third seal can be a portion (e.g., a valve member) of a vent assembly configured to release pressurized gas from the gas chamber. The release of the pressurized gas from the gas chamber can, for example, occur following the delivery of the medicament to facilitate the retraction of the medicament container assembly, a needle, or other delivery member. In such an embodiment, the first seal coupled to the release member can be the movable seal member and the gas chamber can be isolated from the exterior volume by the movable seal member following actuation of the apparatus but prior to the release of the pressurized gas from the gas container. However, in additional embodiments, movable seal member can be the second seal coupled to the carrier. Insofar as the carrier can be configured to move in response to a force exerted by the pressurized gas to insert a needle into a patient, the movement of the carrier can move the movable seal member to the second seal position, thereby isolating the gas chamber from the exterior volume. Further, in some embodiments, as a portion of the vent assembly, the third seal can correspond to the movable seal member. In such embodiments, the force exerted by the pressurized gas can move the third seal from the first seal position to the second seal position, thereby occluding a vent opening of the apparatus. The third seal can be maintained at the second seal position until a movement of an elastomeric member of the medicament container assembly causes the third seal to transition to a third seal position that establishes the gas chamber in fluid communication with the vent opening.

As used herein, the terms “substance” or “medicament” includes any constituent of a therapeutic substance. A medicament can include such constituents regardless of their state of matter (e.g., solid, liquid or gas). Moreover, a medicament can include the multiple constituents that can be included in a therapeutic substance in a mixed state, in an unmixed state and/or in a partially mixed state. A medicament can include both the active constituents and inert constituents of a therapeutic substance. Accordingly, as used herein, a medicament can include non-active constituents such as, water, colorant or the like.

The term “about” when used in connection with a referenced numeric indication means the referenced numeric indication plus or minus up to 10 percent of that referenced numeric indication. For example, “about 100” means from 90 to 110.

In a similar manner, term “substantially” when used in connection with, for example, a geometric relationship, a numerical value, and/or a range is intended to convey that the geometric relationship (or the structures described thereby), the number, and/or the range so defined is nominally the recited geometric relationship, number, and/or range. For example, two structures described herein as being “substantially parallel” is intended to convey that, although a parallel geometric relationship is desirable, some non-parallelism can occur in a “substantially parallel” arrangement. By way of another example, a structure defining a volume that is “substantially 0.50 milliliters (mL)” is intended to convey that, while the recited volume is desirable, some tolerances can occur when the volume is “substantially” the recited volume (e.g., 0.50 mL). Such tolerances can result from manufacturing tolerances, measurement tolerances, and/or other practical considerations (such as, for example, minute imperfections, age of a structure so defined, a pressure or a force exerted within a system, and/or the like). As described above, a suitable tolerance can be, for example, of +10% of the stated geometric construction, numerical value, and/or range. Furthermore, although a numerical value modified by the term “substantially” can allow for and/or otherwise encompass a tolerance of the stated numerical value, it is not intended to exclude the exact numerical value stated.

As used herein, the term “set” can refer to multiple features or a singular feature with multiple parts. For example, when referring to set of walls, the set of walls can be considered as one wall with multiple portions, or the set of walls can be considered as multiple, distinct walls. Thus, a monolithically-constructed item can include a set of walls. Such a set of walls can include, for example, multiple portions that are either continuous or discontinuous from each other. A set of walls can also be fabricated from multiple items that are produced separately and are later joined together (e.g., via a weld, an adhesive, or any suitable method).

As used in this specification and the appended claims, the words “proximal” and “distal” refer to direction closer to and away from, respectively, an operator of the medical device. Thus, for example, the end of the medicament delivery device contacting the patient's body would be the distal end of the medicament delivery device, while the end opposite the distal end would be the proximal end of the medicament delivery device.

As used herein, the terms “stiffness” or “rigidity” relate to an object's resistance to deflection, deformation, and/or displacement produced by an applied force, and is generally understood to be the opposite of the object's “flexibility.” For example, a gas release member with greater stiffness is more resistant to deflection, deformation and/or displacement when exposed to a force than a gas release member having a lower stiffness. Similarly stated, a gas release member having a higher stiffness can be characterized as being more rigid than a gas release member having a lower stiffness. Stiffness can be characterized in terms of the amount of force applied to the object and the resulting distance through which a first portion of the object deflects, deforms, and/or displaces with respect to a second portion of the object. When characterizing the stiffness of an object, the deflected distance may be measured as the deflection of a portion of the object different than the portion of the object to which the force is directly applied. Said another way, in some objects, the point of deflection is distinct from the point where force is applied.

Stiffness (and therefore, flexibility) is an extensive property of the object being described, and thus is dependent upon the material from which the object is formed as well as certain physical characteristics of the object (e.g., cross-sectional shape, length, boundary conditions, etc.). For example, the stiffness of an object can be increased or decreased by selectively including in the object a material having a desired modulus of elasticity, flexural modulus and/or hardness. The modulus of elasticity is an intensive property of (i.e., is intrinsic to) the constituent material and describes an object's tendency to elastically (i.e., non-permanently) deform in response to an applied force. A material having a high modulus of elasticity will not deflect as much as a material having a low modulus of elasticity in the presence of an equally applied stress. Thus, the stiffness of the object can be decreased, for example, by introducing into the object and/or constructing the object of a material having a relatively low modulus of elasticity.

The stiffness of an object can also be increased or decreased by changing a physical characteristic of the object, such as the shape or cross-sectional area of the object. For example, an object having a length and a cross-sectional area may have a greater stiffness than an object having an identical length but a smaller cross-sectional area. As another example, the stiffness of an object can be reduced by including one or more stress concentration risers (or discontinuous boundaries) that cause deformation to occur under a lower stress and/or at a particular location of the object. Thus, the stiffness (or flexibility) of the object can be decreased by decreasing and/or changing the shape of the object.

Thus, an object that deforms readily under small forces, such as, for example, a wire, a filament, a cord, or the like is said to be a flexible object.

The therapeutic compositions described herein can be included in any suitable medicament delivery device as described herein or in International Patent Publication No. WO2017/004345, entitled “Auto-Injectors for Administration of a Medicament Within a Prefilled Syringe,” filed Jun. 30, 2016 (“the '4345 PCT”), International Patent Publication No. WO2020/140040, entitled “Devices and Methods for Delivery of Substances Within a Prefilled Syringe,” filed Dec. 27, 2019 (“the '0040 PCT”), International Patent Publication No. WO2018/136413, entitled “Medicament Delivery Devices with Wireless Connectivity and Event Detection,” filed Jan. 16, 2018 (“the '6413 PCT”), and/or WO2020/018433, entitled “Medicament Delivery Devices with Wireless Connectivity and Compliance Detection,” filed Jul. 15, 2019 (“the '8433 PCT”), cach of which is incorporated herein by reference in its entirety. For example, in some embodiments, a drug product configured for administration by an untrained user (such a person accompanying the patient) can include a dose of icatibant. Such drug products can include, for example, an auto-injector having a needle length and delivery profile (e.g., flow of the icatibant) sufficient to produce subcutaneous injection. In other embodiments, a drug product can include a therapeutic substance including of a monoclonal antibody. Such drug products can include, for example, an auto-injector having multiple prefilled syringe containers and that delivers the medicament from each of the syringes in one operation to deliver the desired dose. By including multiple syringes, such arrangements can allow for higher doses while still using a standard fill volume within the prefilled syringe.

In some embodiments, a gas-powered medicament delivery device can result in a compact device, in which the outer dimensions of the housing are not substantially larger than the length of the medicament container disposed therein. For example, as shown and described herein, in some embodiments, a medicament delivery device can be devoid of a mechanical linkage that exerts or transfers a force to an elastomeric member to expel a medicament from a medicament container therein. Similarly stated, in some embodiments, a medicament delivery device can be devoid of mechanical linkages (rams, rods) that transfer force to the elastomeric member. Rather, in some embodiments, the elastomeric member can exert a force onto a member (e.g., an expandable member) to provide control over the delivery. Such medicament delivery devices (or medicament delivery mechanisms) are considered to be “pistonless” systems. As one example, in a pistonless, gas-powered auto-injector, the force exerted by the gas can move the medicament container relative to the housing and similarly, can move the elastomeric member relative to (e.g., within) the medicament container. In some embodiments, by not including a movable mechanism, a piston, and/or the like, a height of the medical injector can be reduced relative to, for example, the height of a device that includes a rigid, single length piston.

For example, any of the medicament delivery devices described herein can include any suitable “pistonless” design, such as those described in the '4345 PCT, the '0040 PCT, or in International Patent Publication No. WO 2016/154427, entitled “DEVICES AND METHODS FOR DELIVERING A LYOPHILIZED MEDICAMENT,” filed on Mar. 24, 2016, which is incorporated herein by reference in its entirety.

In some embodiments, the characteristics of the medicament, the medicament container and the needle are such that the force required to achieve the desired injection is not possible via manual injection. Accordingly, in some embodiments a device can include an energy storage member configured to produce the desired force (and/or pressure within the medicament container) to deliver the medicament. For example, in certain circumstances, the pressure of the medicament within a needle-based medicament container can be modeled by the Hagen-Poiseuille law, as indicated below:

P=(8*μ*L*Q)/(Π*R4) (1)

where P is the pressure of the medicament within the medicament container, μ is the viscosity of the medicament, L is the length of the needle (not shown), Q is the flow rate of the medicament through the needle, and R is the radius of the lumen defined by the needle. Because the pressure (and/or force) required to inject a high viscosity fluid through a small-bore needle is proportional to the inverse of the radius of the lumen of the needle to the fourth power, the pressure of the medicament within the medicament container necessary to achieve the desired flow rate can, at times, be relatively high. By including a gas-based energy storage member, the desired pressure can be achieved.

In some embodiments, the energy storage member can be configurable to include various amounts of stored energy without changing the size of the energy storage member. In such embodiments, therefore, a high force (e.g., to inject viscous medicaments) can be achieved in the same packaging that is used for lower viscosity medicaments. For example, in some embodiments, the energy storage member can be a compressed gas cylinder having any desired pressure (and thus, mass) of gas therein. Accordingly, the pressure and/or force can be achieved to complete the operations described herein, regardless of the medicament.

In such embodiments, the use of a non-mechanical energy storage member (e.g., gas, propellant, or the like) can produce a sufficiently high force to produce the desired pressure within the medicament container to produce the desired injection. For example, in such embodiments having a larger diameter, the amount of force needed to produce a desired internal pressure increases significantly. In some embodiments, any of the medicament delivery devices shown herein can include a gas-based energy storage system configured to produce a gas pressure (e.g., within the gas chamber) of between about 200 psi and about 2700 psi. In some embodiments, any of the injectors shown herein can include a gas-based energy storage system configured to produce a gas pressure of about 200 psi, 300 psi, 400 psi, 500 psi, 600 psi, 700 psi, 800 psi, 900 psi, 1100 psi, 1200 psi, 1300 psi, 1500 psi, 1700 psi, 1900 psi, 2100 psi, 2300 psi, 2500 psi, or 2700 psi. In some embodiments, any of the injectors shown herein can include a gas-based energy storage system configured to produce a gas pressure of between about 200 psi to 7000 psi. The gas pressure can be produced by any suitable mechanism, such as, for example, by puncturing a compressed gas container, releasing a propellant (e.g., hydrofluoroalkane), releasing a refrigerant (e.g., R134a), releasing a liquefied gas, triggering a chemical reaction, or the like.

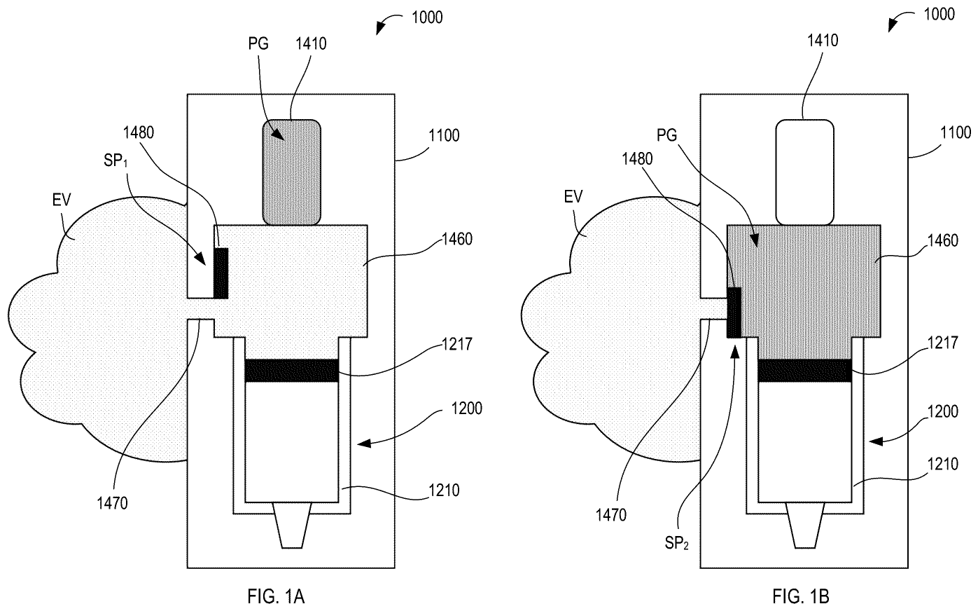

FIGS. 1A and 1B are schematic illustrations of an apparatus (e.g., medicament delivery device) 1000 according to an embodiment. As depicted, the apparatus 1000 includes a housing 1100, a gas container 1410, a medicament container assembly 1200, and a movable seal member 1480, and is surrounded by an exterior volume EV. The housing 1100 defines a gas chamber 1460 and an equalization bypass 1470. The gas chamber 1460 can, for example, the portion of the volume defined by the housing 1100 within which a portion of the medicament container assembly 1200 is disposed. The housing 1100 can be any suitable size, shape, or configuration and can be made of any suitable material. For example, in some embodiments, the housing 1100 is an assembly of multiple parts formed from a plastic material and defines a substantially rectangular shape when assembled. In other embodiments, the housing 1100 can have a substantially cylindrical shape.

The gas container 1410 is disposed within the housing 1100. The gas container 1410 is configured to produce a pressurized gas PG within the gas chamber 1410 when the apparatus 1000 is actuated. Said another way, the gas container 1410 can be configured to convey the pressurized gas into the gas chamber 1460 to produce a force to convey the contents of the medicament container assembly 1200 when the apparatus is actuated. The gas container 1410 can be any suitable member or device that stores potential energy and, when actuated, produces the pressurized gas. For example, the gas container 1410 (and any of the gas containers described herein) can be any of a device containing compressed gas, a device containing a vapor pressure-based propellant or the like. For example, the gas container 1410 can be a sealed gas container containing a volume of inert gas at a pressure that is at least 900 psi (e.g., 1,000 psi or greater).

The medicament container assembly 1200 has a medicament container body 1210 that defines a volume that contains (i.e., is filled with or partially filled with) a medicament. The distal end portion of the medicament container body 1210 includes a neck or opening through which the medicament can be delivered. In some embodiments, the medicament container assembly 1200 can include a delivery member (e.g., a nozzle, a needle, a delivery orifice, a mouthpiece and/or other similar structure) coupled to the container body 1210 through which the medicament is delivered. For example, in some embodiments, the medicament container assembly 1200 can be a prefilled syringe having a needle staked thereto, of the types shown and described herein. In other embodiments, the medicament container assembly 1200 can be a cartridge that is sealed and that can be selectively coupled to a needle upon actuation of the apparatus. For example, in some embodiments, the medicament container assembly 1200 can be similar to the multi-chamber cartridges shown and described in U.S. Provisional Application No. 63/398,410, entitled “Devices and Methods for Delivering Reconstituted Medicaments,” filed Aug. 16, 2022, which is incorporated herein by reference in its entirety. In some embodiments, the medicament container assembly 1200 can include or be coupled to a carrier (not shown, but which can be similar to the carrier 4360 described below) that moves the medicament container body 1210 within the housing 1100. In this manner, the carrier can facilitate moving the delivery member out of the housing 1100 in a deployed position, as described below. In some embodiments, one or more surfaces of the carrier can form at least a portion of a boundary of the gas chamber 1460. In some embodiments, the carrier can include one or more seals to fluidically isolate the gas chamber 1460.

The medicament container assembly 1200 includes an elastomeric member 1217 that seals the medicament within the container body 1210. The elastomeric member 1217 is configured to move within the container body to inject the medicament from the medicament container assembly 1200. The elastomeric member 1217 can be of any design or formulation suitable for contact with the medicament. For example, the elastomeric member 1217 can be formulated to minimize any reduction in the efficacy of the medicament that may result from contact (either direct or indirect) between the elastomeric member 1217 and the medicament. For example, in some embodiments, the elastomeric member 1217 can be formulated to minimize any leaching or out-gassing of compositions that may have an undesired effect on the medicament. In other embodiments, the elastomeric member 1217 can be formulated to maintain its chemical stability, flexibility and/or sealing properties when in contact (either direct or indirect) with the medicament over a long period of time (e.g., for up to six months, one year, two years, five years or longer).

The medicament container assembly 1200 can include a proximal portion configured to translate within the housing 1100 to move the medicament container body 1210 between positions as described herein. Although the medicament container assembly 1200 is shown as being disposed within the housing 1100 without a carrier, in other embodiments, the medicament container assembly 1200 can be disposed within or coupled to a carrier to facilitate movement within the housing 1100. The proximal portion of the medicament container assembly 1200 and the carrier (if present) can define a portion of a boundary of the gas chamber 1460. In this manner, when a pressurized gas is conveyed into the gas chamber 1460, the pressure therein can produce a force on the medicament container assembly 1200 to move the medicament container body 1210, for example, from a withdrawn position to a deployed position.

In some embodiments, the apparatus 1000 can include a retraction member (not shown). The retraction member can be a retraction spring or any other energy accumulation member. In this manner, the retraction member can be configured to move the medicament container assembly 1200 back towards the withdrawn position after it has been deployed as described in further detail herein. In some embodiments, the retraction member can further be configured to maintain the medicament container 1200 in the withdrawn position prior to gas pressure being supplied to the gas chamber 1460. In some embodiments, the apparatus 1000 can be configured to maintain the medicament container patentably 1200 in the deployed position after delivery. In such embodiments, the apparatus 1000 can include other suitable mechanisms for covering or shielding the delivery member (e.g., a cover that moves about the delivery member after delivery is completed).

As depicted in FIG. 1A, prior to actuation, the gas chamber 1460 is in fluid communication with the exterior volume EV via the equalization bypass 1470. Due to the fluid communication provided by the equalization bypass 1470, the pressure within the gas chamber 1460 corresponds to the pressure of the exterior volume EV. Accordingly, the fluid communication between the gas chamber 1460 and the exterior volume EV via the equalization bypass 1470 precludes the development of a substantial pressure differential between the gas chamber 1460 and the exterior volume EV that might otherwise affect a condition and/or operation of the apparatus 1000. Similarly stated, the equalization bypass 1470 precludes the development of a substantial pressure differential across the elastomeric member 1217, which could result in undesired movement or sticking of the elastomeric member 1217 within the container body 1210. However, as it may be desirable to increase the pressure within the gas chamber 1460, the apparatus 1000 includes the movable seal member 1480 to selectively isolate the gas chamber 1460 from the exterior volume EV. FIG. 1A depicts the movable seal member 1480 at the first seal position SP₁. When the movable seal member 1480 is at the first seal position SP₁, the gas chamber 1460 is in fluid communication with the exterior volume EV via the equalization bypass 1470. Following actuation of the apparatus 1000, the movable seal member 1480, as depicted in FIG. 1B, is at a second seal position SP₂, and the gas chamber 1460 is fluidically isolated from the exterior volume EV. With the gas chamber 1460 fluidically isolated from the exterior volume by the movable seal member 1480 at the second seal position, the pressurized gas PG within the gas chamber 1460 exerts a force on the elastomeric member 1217 to cause the desired movement of the elastomeric member 1217 within the container body to dispense a portion of the medicament from the medicament container assembly 1200. As described herein, in some embodiments, the movable seal member 1480 can transition from the first seal position SP₁ to the second seal position SP₂ following actuation of the apparatus 1000 but prior to the introduction of the pressurized gas PG into the gas chamber 1460. In additional embodiments, the force exerted by the pressurized gas PG in the gas chamber 1460 can transition the movable seal member 1480 from the first seal position SP₁ to the second seal position SP₂ to isolate the gas chamber 1460 from the exterior volume EV.

When the apparatus 1000 is actuated, the gas container 1410 is activated and releases pressurized gas PG into the gas chamber 1460. In some embodiments, the released pressurized gas produces a force that moves the medicament container body 1210 together with the delivery member from a withdrawn position to a deployed position. The delivery member is disposed within the housing 1100 in the withdrawn position and the delivery member extends out of the housing 1100 in the deployed position. In some embodiments, the pressurized gas PG is introduced to the gas chamber 1460. Accordingly, once the gas chamber 1460 is isolated from the exterior volume EV as depicted in FIG. 1B, the pressure builds within the gas chamber 1460. After the pressure in the medicament gas chamber 1460 overcomes resistance of the elastomeric member 1217 against an interior of the medicament container body 1210, the elastomeric member 1217 moves away from the gas chamber 1460, thereby expelling the medicament from within the medicament container body 1210.

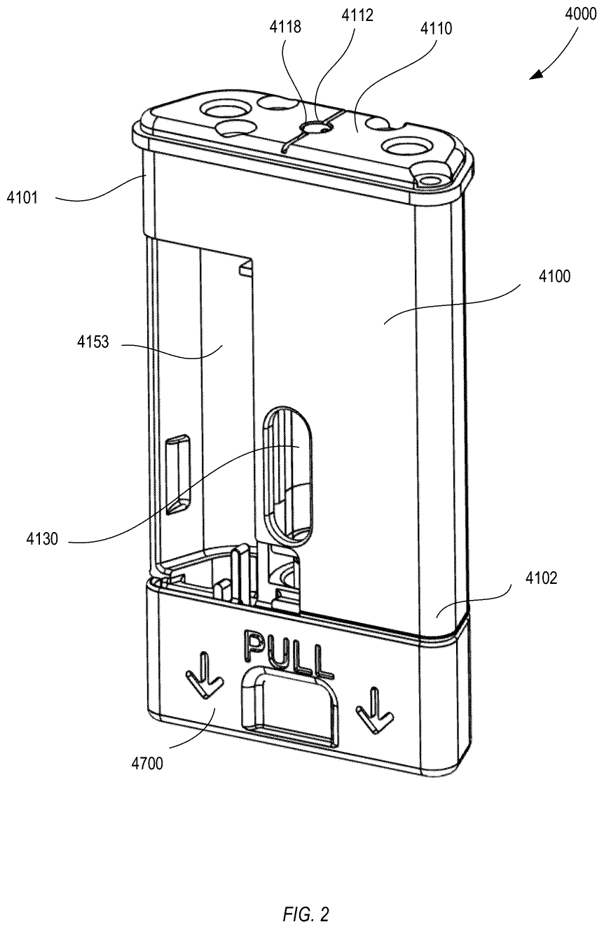

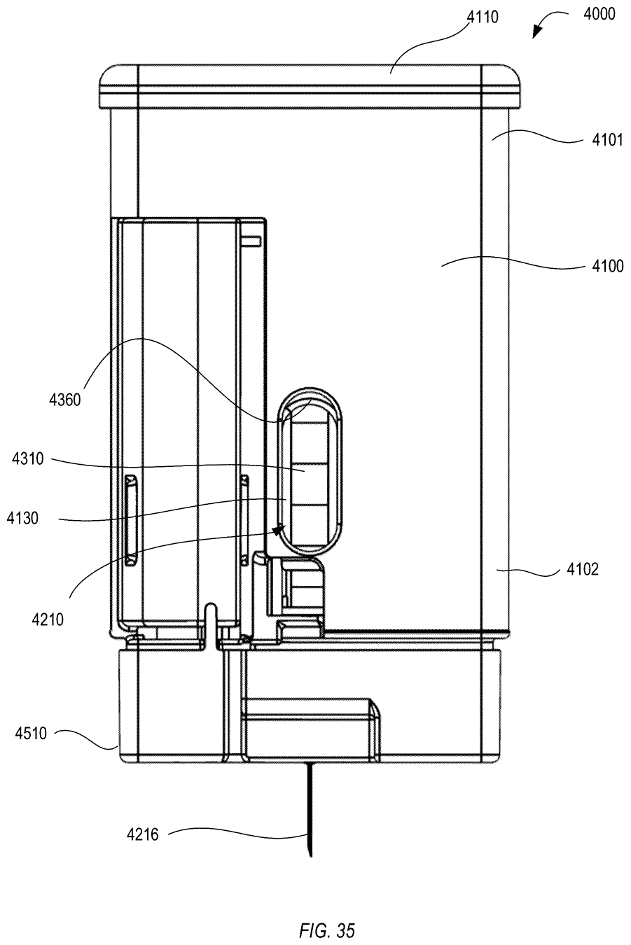

In some embodiments, a medicament delivery device can be an auto-injector having a pistonless delivery system in which the force exerted by the gas can move the medicament container relative to the housing and the elastomeric member relative to (e.g., within) the medicament container. For example, FIGS. 2-40 show a medical injector 4000 (also referred to as “auto-injector,” “injector,” or “device”), according to an embodiment. The medical injector 4000 is a gas-powered auto-injector configured to deliver a medicament contained within a prefilled syringe 4200, as described herein. In some embodiments, the medical injector 4000 can be configured to be affixed to the patient as an on-body system. A discussion of the components of the medical injector 4000 will be followed by a discussion of the operation of the medical injector 4000. Certain aspects of the medical injector 4000 can be similar to or substantially the same to the medical injectors described in the '4345 PCT, the '0040 PCT, the '6413 PCT, U.S. patent application Ser. No. 13/357,935 (now U.S. Pat. No. 9,084,849) entitled, “MEDICAMENT DELIVERY DEVICES FOR ADMINISTRATION OF A MEDICAMENT WITHIN A PREFILLED SYRINGE,” filed on Jan. 25, 2012 (referred to henceforth as the “'849 patent”), the disclosures of each of which are incorporated herein by reference in its entirety.

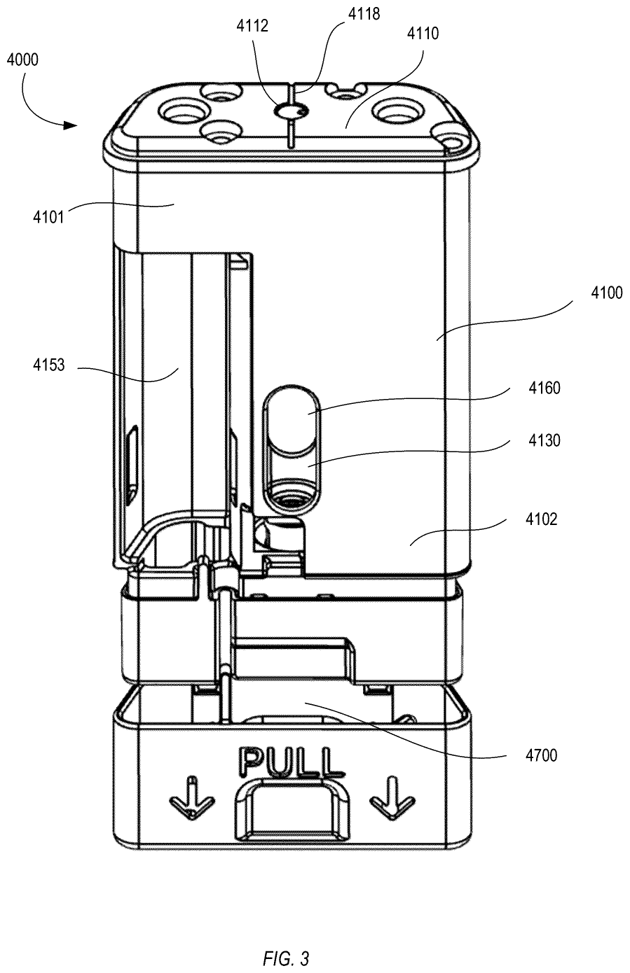

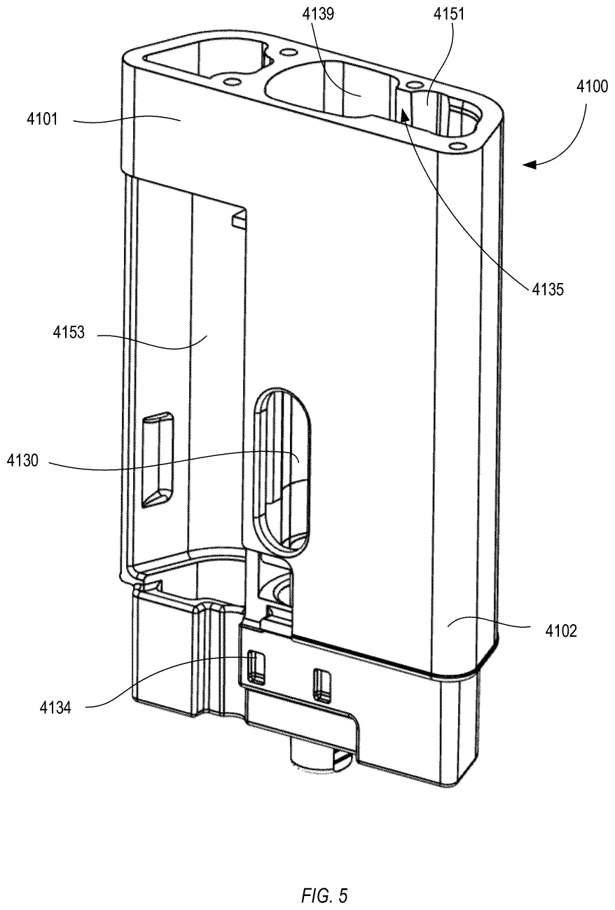

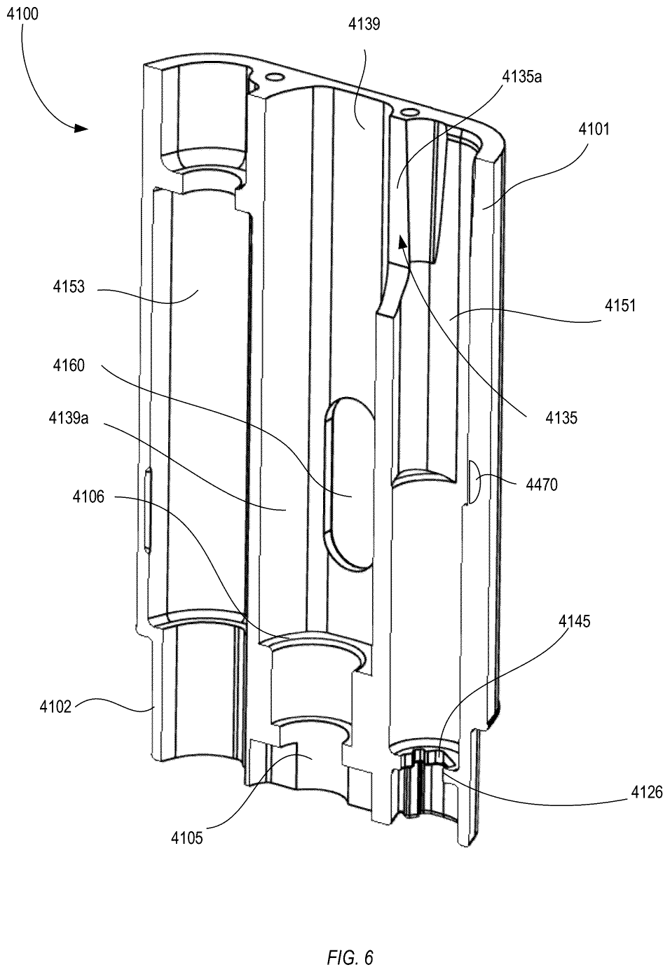

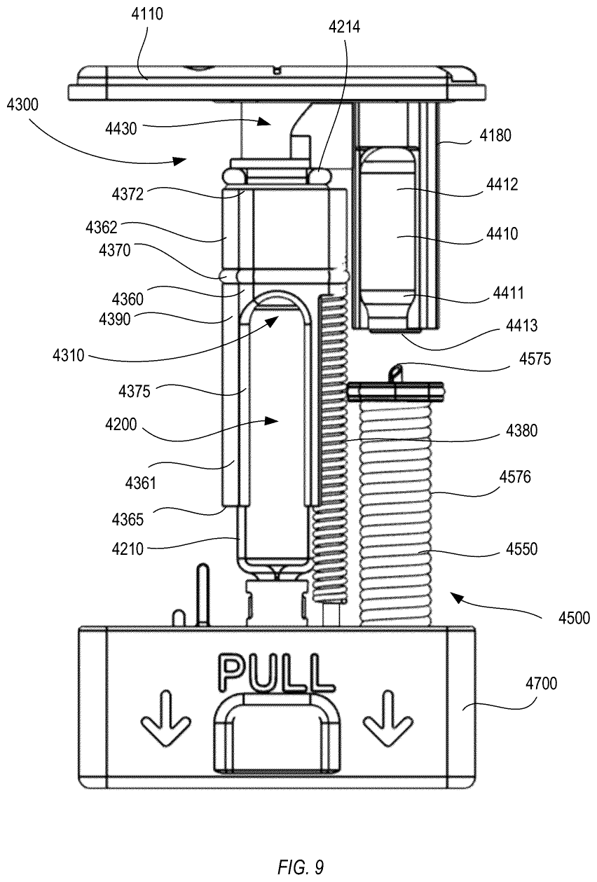

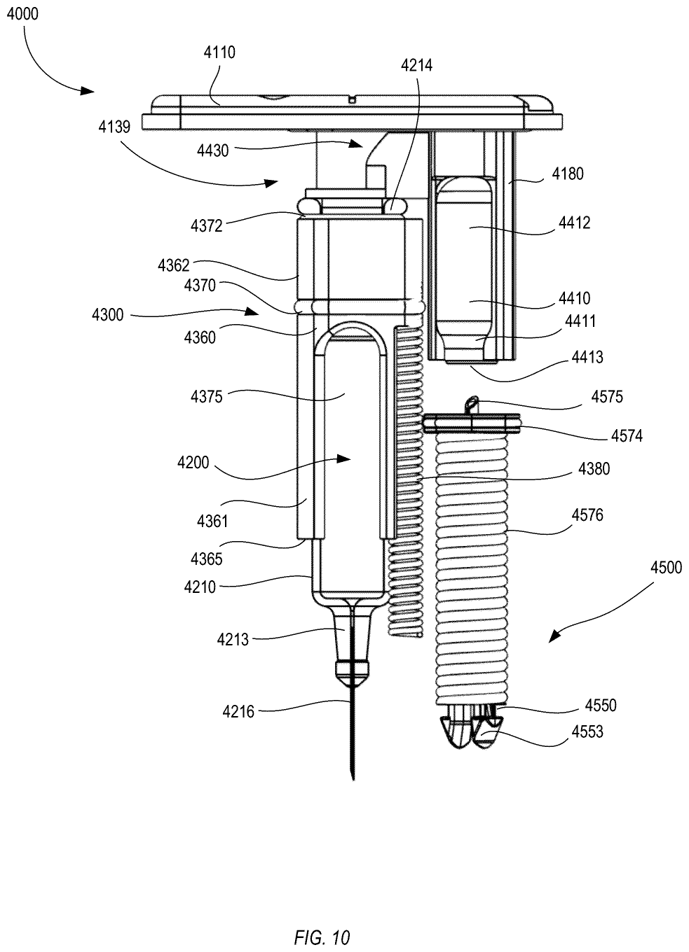

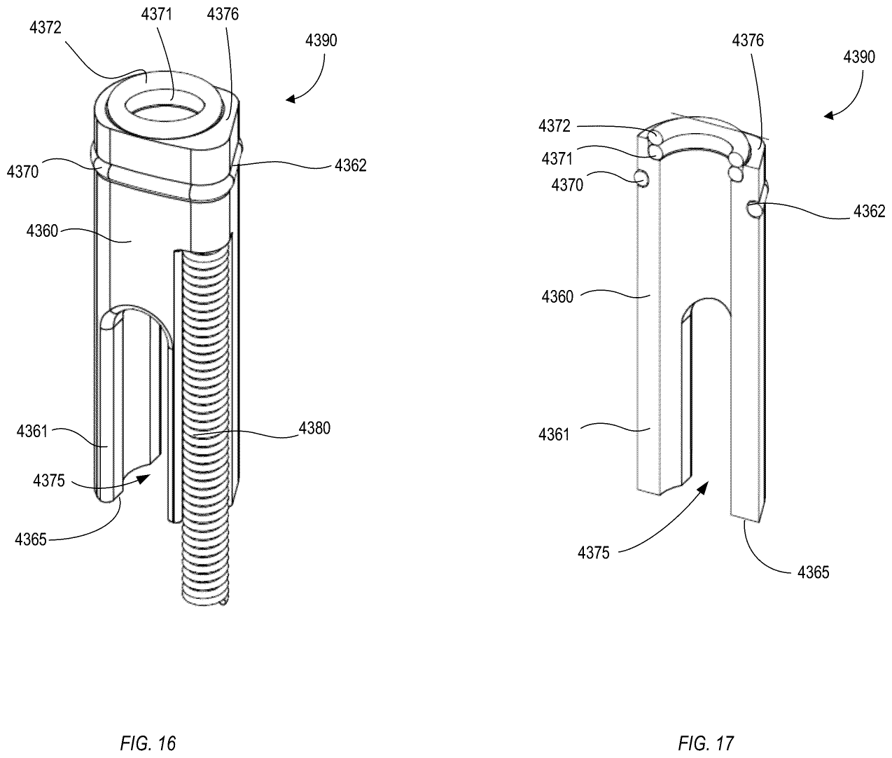

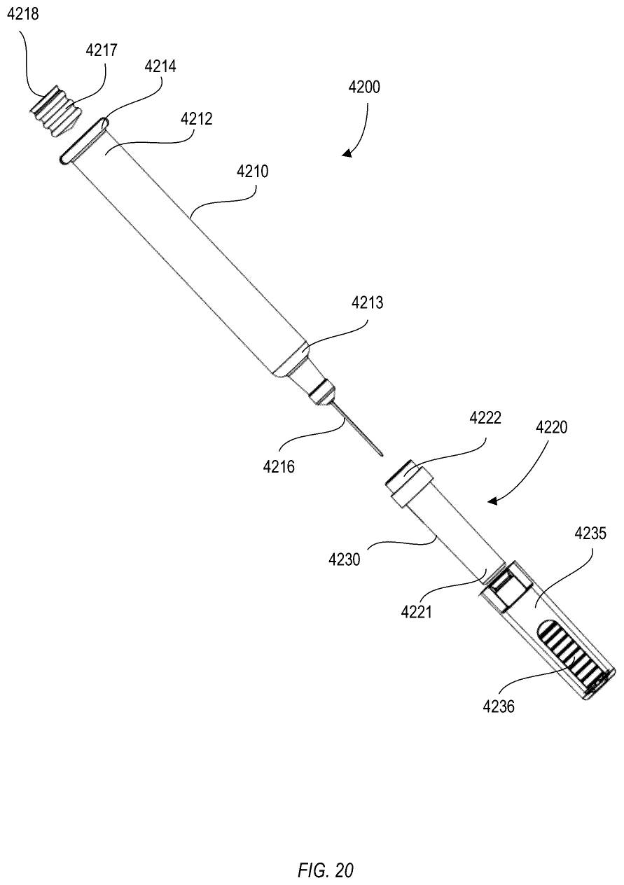

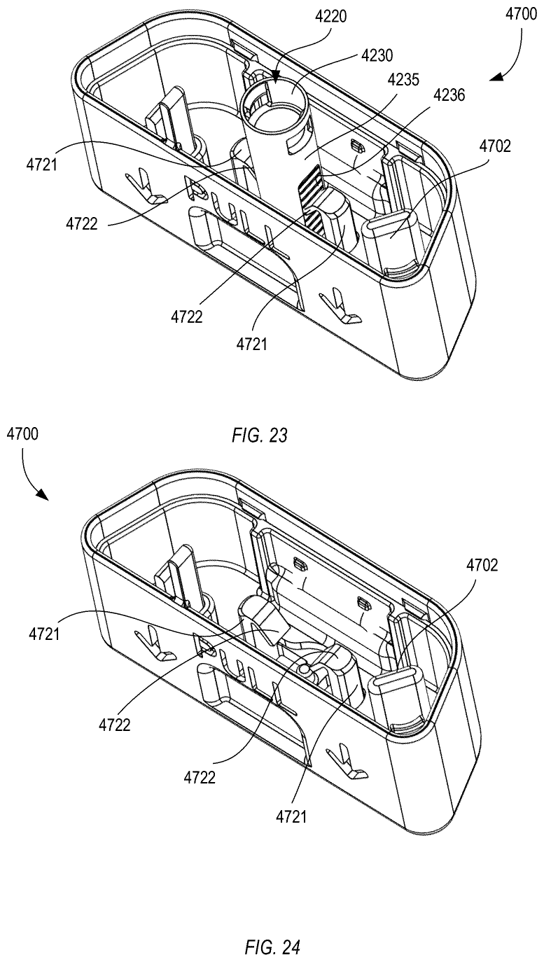

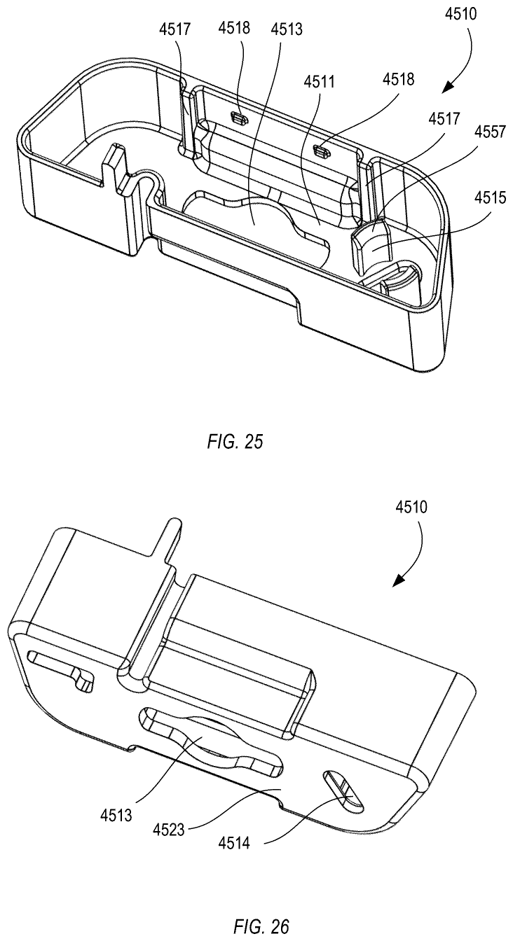

The medical injector 4000 includes a housing 4100 (sec e.g., FIGS. 5 and 6), a system actuation assembly 4500 (sec e.g., FIGS. 9 and 10), a medicament container assembly 4200 (see FIG. 20), a medicament delivery mechanism 4300 (see e.g., FIGS. 16-20), a base 4510 (or actuator, sec FIGS. 25 and 26); and a safety lock 4700 (see FIGS. 23 and 24). As shown in FIGS. 3-6, the housing 4100 has a proximal end portion 4101 and a distal end portion 4102. The housing 4100 defines a first status indicator aperture 4130 and a second status indicator aperture 4160. The first status indicator aperture 4130 defined by the housing 4100 is located on a first side of the housing 4100, and the second status indicator aperture 4160 of the housing 4100 is located on a second side of the housing 4100. The status indicator apertures 4130, 4160 can allow a patient to monitor the status and/or contents of the medicament container 4200, the carrier 4360, and the medicament contained within the housing 4100. For example, by visually inspecting the status indicator apertures 4130, 4160, a patient can determine whether the medicament container 4200 contains a medicament and/or whether the medicament has been dispensed.

As shown in FIGS. 2, 3, and 5, the housing 4100 includes an electronic circuit system cavity 4153 that can house any of the electronics described herein, and/or any of the electronic circuit systems described in the '8433 PCT. Although the housing 4100 is shown with an electronic circuit system cavity 4153, in some embodiments, the medical injector 4000 need not include any electronics or the electronic circuit system cavity 4153. In some embodiments, the housing 4100 can include a label or indicia that mask or otherwise accentuates the status indicator apertures 4130, 4160 and/or the contents viewed therethrough. For example, in some embodiments, the housing 4100 can include a label (not shown) having border that surrounds at least a portion of the status indicator aperture 4130, the status indicator apertures 4160 (or both). In some embodiments, a label can include indicator colors that alert user (or assist a user in determining) whether the medicament is properly colored, whether a portion of the carrier 4360 is visible through the window or the like.

As shown in FIGS. 5, 6 and 12, the housing 4100 defines a gas container cavity 4151 and a medicament cavity 4139. The gas container cavity 4151 is configured to receive the gas container 4410 and a portion of the system actuator assembly 4500 (e.g., a release member 4550 and the spring 4576, as shown in FIGS. 9 and 10). The proximal end portion of the gas container cavity 4151 is configured to receive the gas container retention member 4180 of a proximal cap 4110 of the housing 4100, as described in further detail herein. The gas container cavity 4151 is in fluid communication with the medicament cavity 4139 via a gas passageway 4135 defined in the housing 4100, as described in further detail herein.

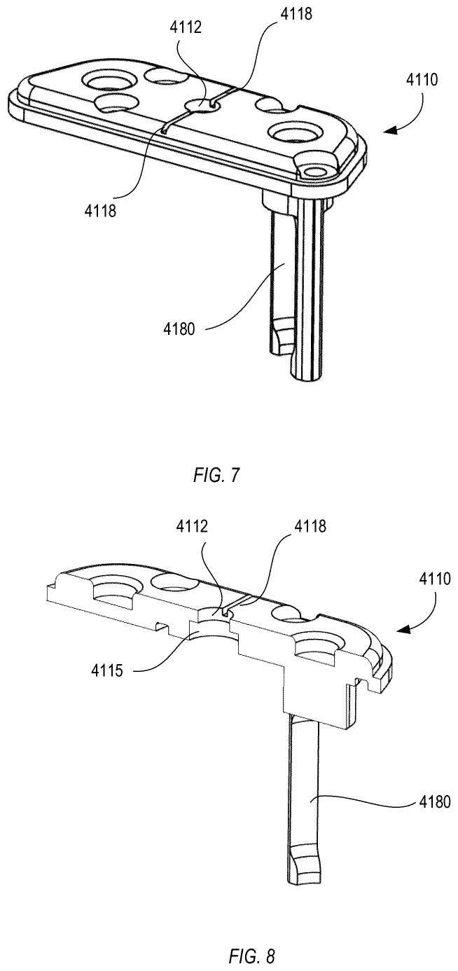

The medicament cavity 4139 is configured to receive the medicament container assembly 4200 and at least a portion of the medicament delivery mechanism 4300. In particular, as described below, the medicament delivery mechanism 4300 includes a carrier assembly 4390 (see e.g., FIGS. 9, 10 and 16-19) and a gas vent assembly 4310 see e.g., FIGS. 7, 8, and 13A, 21 and 22A-22C) movably disposed in the medicament cavity 4139. The medicament cavity 4139 is in fluid communication with a region outside the housing 4100 via a needle aperture 4105 and also a vent opening 4112. It should be appreciated that while a single medicament container assembly is depicted, in additional embodiments, additional medicament container assemblies 4200 can be employed. For example, in some embodiments, the housing 4100 can include two medicament cavities 4139 with each of the two medicament cavities 4139 containing an individual medicament container assembly 4200.

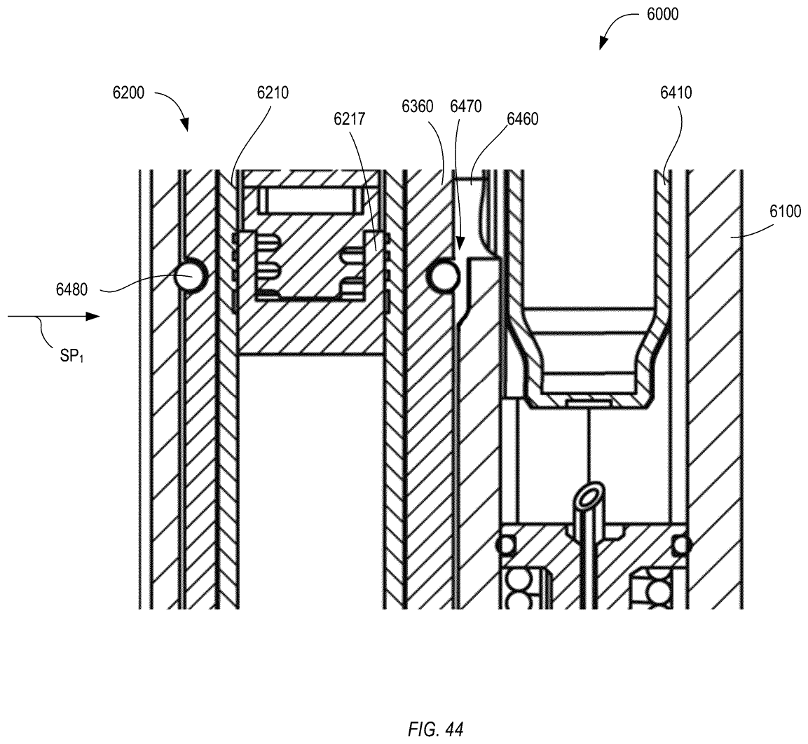

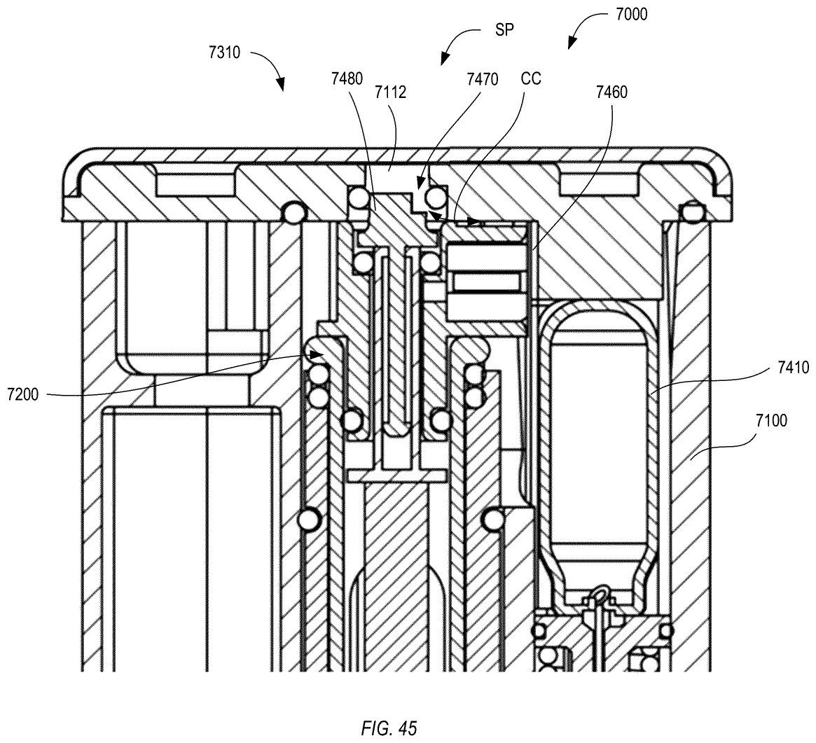

The proximal end portion 4101 of the housing 4100 includes a proximal cap 4110 (see e.g., FIGS. 7, 8, and 13). The proximal cap 4110 includes a gas container retention member 4180 configured to receive and/or retain a gas container 4410 that contains a pressurized gas, as shown in FIGS. 7-10. When the medical injector 4000 is actuated, pressurized gas from the gas container 4410 is conveyed from the gas container cavity 4151 to the medicament cavity 4139 via the gas passageway 4135 of the housing 4100. Said another way, the gas passageway 4135 places the gas container cavity 4151 in fluid communication with the medicament cavity 4139. Thus, the proximal portion of the medicament cavity 4139 can be referred to as (or can function as) a housing gas chamber (e.g., gas chambers 6460, 7460 as depicted in FIGS. 44 and 45). Similarly stated, the proximal portion of the medicament cavity 4139 is a volume within which a pressurized gas is conveyed to move the carrier 4360 and to serve as a pressurized gas reservoir used to inject the medicament, as described herein.

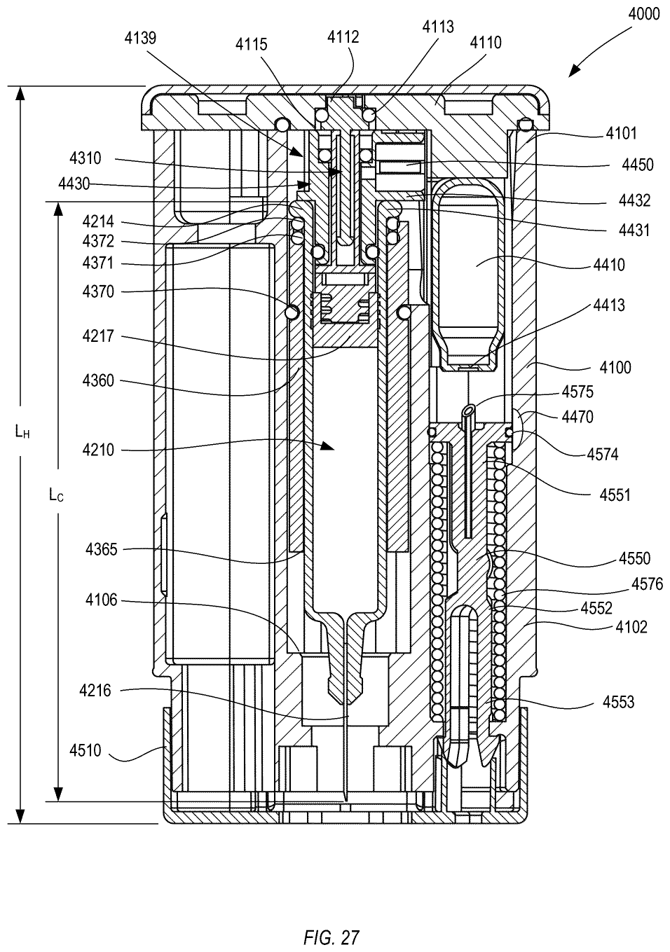

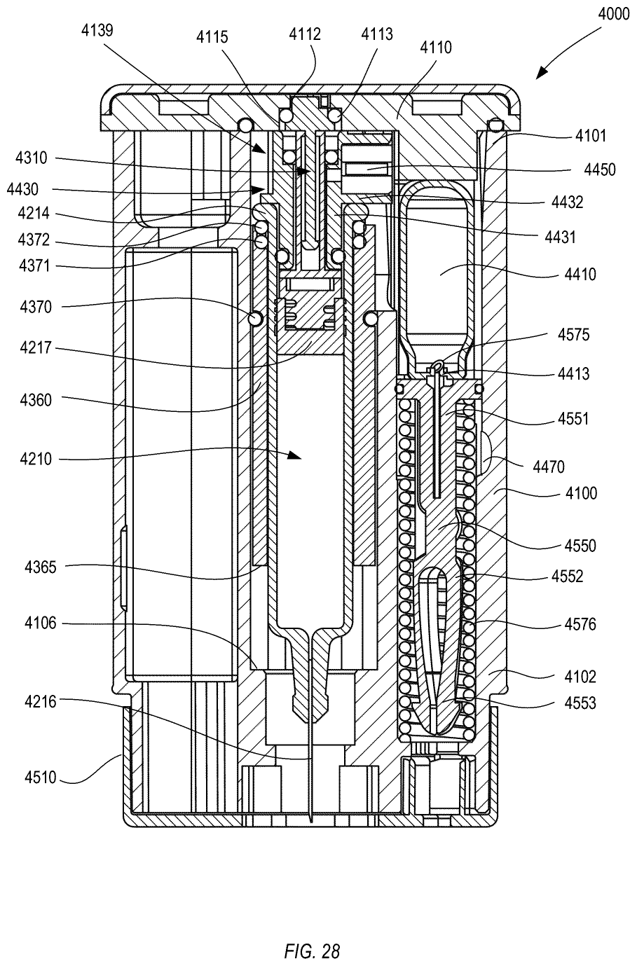

As depicted in FIGS. 6, 12, 27, 28, 30, 31, 33, 34, 36, 37, 39, and 40, in some embodiments, the housing 4100 defines an equalization bypass 4470. The equalization bypass 4470 facilitates a fluidic coupling between the proximal portion of the medicament cavity 4139 and an exterior volume surrounding the housing. As depicted in FIG. 27, when a movable seal member, such as sealing member 4574, is at a first seal position, the proximal portion of the medicament cavity 4139 is in fluid communication with the exterior volume via the equalization bypass 4470. However, as depicted in FIG. 28, when the medical injector is actuated, the movable seal moves from the first seal position to a second seal position. When the movable seal member is at the second seal position, the proximal portion of the medicament cavity 4139 is fluidically isolated from the exterior volume.

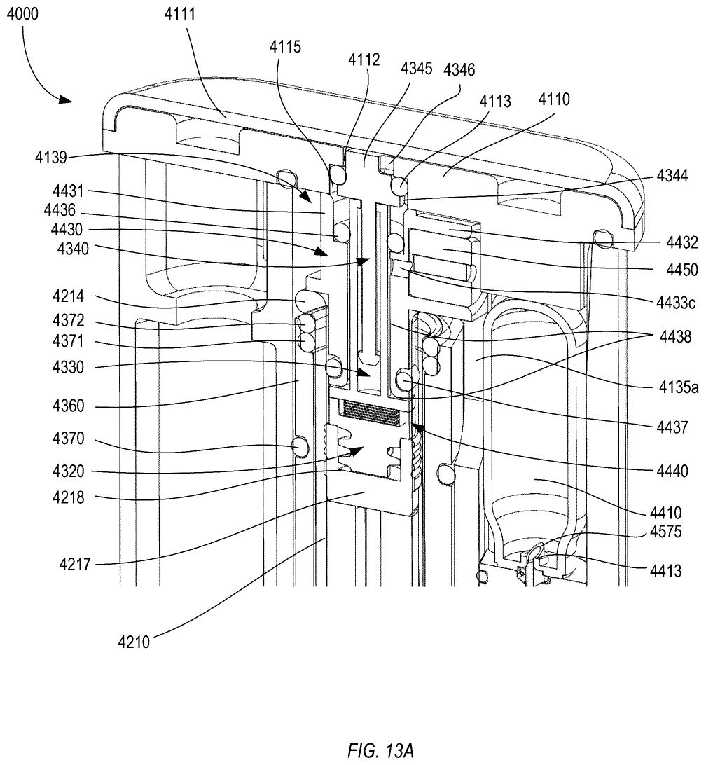

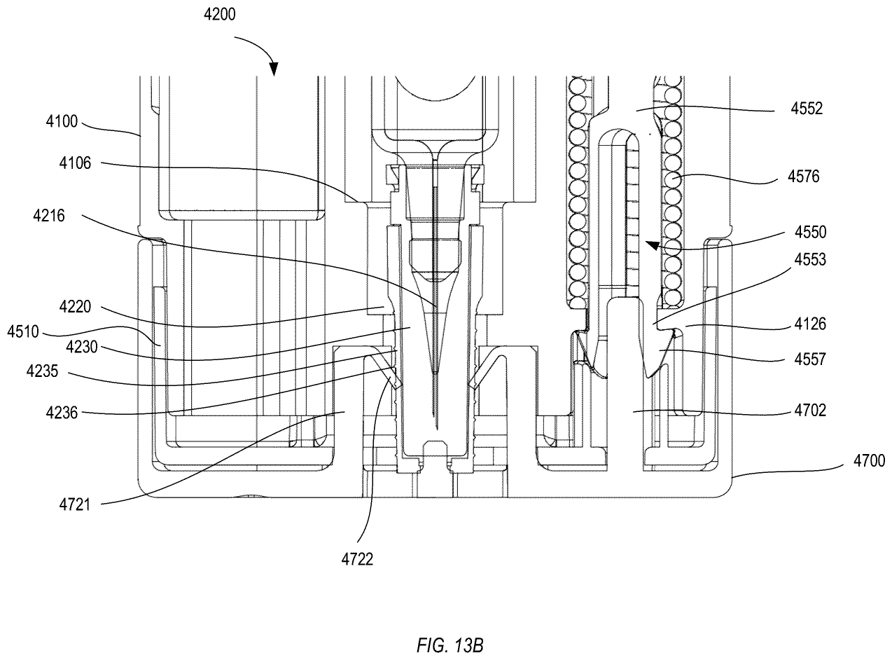

The proximal cap 4110 also includes a cap cover 4111 coupled to a proximal end portion of the proximal cap 4110 while retaining a gap 4111a between the proximal end portion of the proximal cap 4110 and the cap cover 4111. The cap cover 4111 prevents the vent opening 4112 from direct external contact and prevents clogging from external debris. The proximal cap 4110 also includes an O-ring 4113 and defines the vent opening 4112. The vent opening 4112 provides the passageway through which pressurized gas is conveyed from the medicament cavity 4139 (or housing gas chamber portion of the medicament cavity 4139) to a volume outside of the medical injector 4000. As shown in FIGS. 7 and 8, the proximal end portion of the proximal cap 4110 defines vent channels 4118 extending laterally away from the vent opening 4112. Together with the gap 4111a, the vent channels 4118 form multiple vent passageways that allow pressurized gas from within the medicament cavity 4139 to escape out to the volume outside the medical injector 4000. In this manner, the force produced by the pressurized gas on the medicament delivery mechanism 4300 and/or the medicament container assembly 4200 can be reduced to allow needle retraction after the injection is completed. As shown in FIG. 13A, the O-ring 4113, in conjunction with the valve portion 4345 of the gas vent assembly 4310, selectively seals the vent opening 4112 during needle insertion and delivery of the medicament.

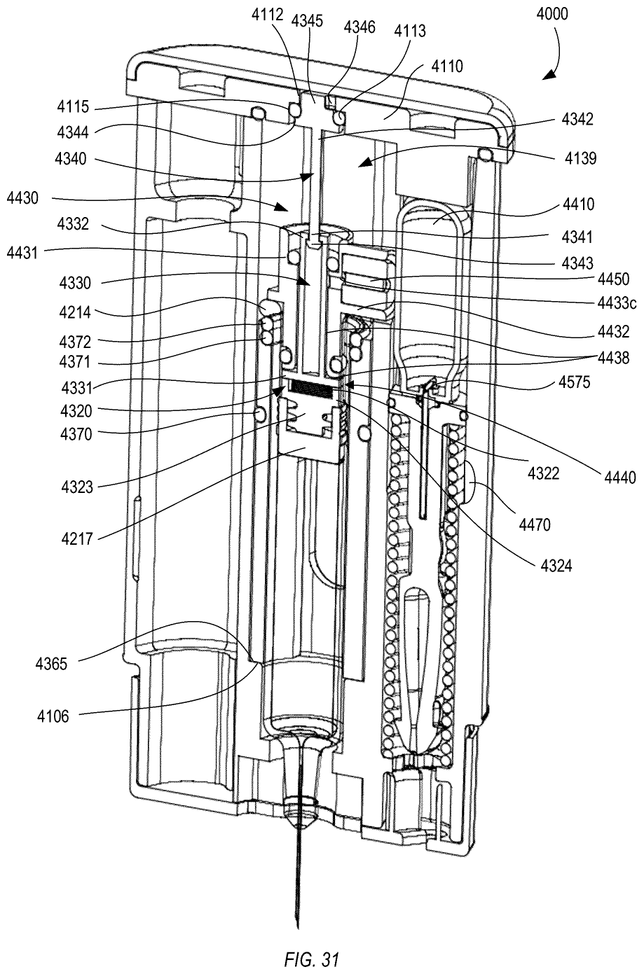

Although the vent opening 4112 is shown as being defined by the proximal cap 4110, and being in a proximal surface thereof, in other embodiments, the vent opening 4112 (and any of the vent openings described herein) can be defined within any suitable portion of the proximal cap or side wall. For example, in some embodiments, the vent opening 4112 (and any of the vent openings described herein) can be defined by the proximal cap 4110 but can have a centerline that is nonparallel to a longitudinal axis of the medical injector 4000. Said another way, in some embodiments, the vent opening 4112 (and any of the vent openings described herein) can open towards a side of the medical injector, rather than opening towards the proximal end, as shown. In other embodiments, the vent opening 4112 (and any of the vent openings described herein) can be defined by any wall and/or surface of the housing 4100. As depicted in FIG. 31 for example, in some embodiments, the medical injector includes both the vent opening 4112 and the separate equalization bypass 4470. This arrangement can allow for improved operational flexibility. For example, in some embodiments, the equalization bypass 4470 can be transitioned from an opened configuration to a closed configuration when the device is actuated. This sequence can allow the medicament cavity 4139 (and the medicament container gas chamber located above the elastomeric member 4217) to be maintained a pressure that is substantially equal to the atmospheric pressure prior to actuation. Maintaining this pressure equalization can preclude the development of a substantial pressure differential across the elastomeric member 4217. In response to actuation, the equalization bypass 4470 can be closed, thereby allowing the gas pressure to build up within the medicament cavity 4139 (and the medicament container gas chamber), to allow needle insertion followed by movement of the elastomeric member 4217 to deliver the medicament. Conversely, the vent opening 4112 can be while the vent opening 4112 can be transitioned from a closed configuration to an opened configuration after the medicament delivery. This causes the pressurized gas to be released from the medicament cavity 4139, thereby enabling retraction of the needle, as described below.

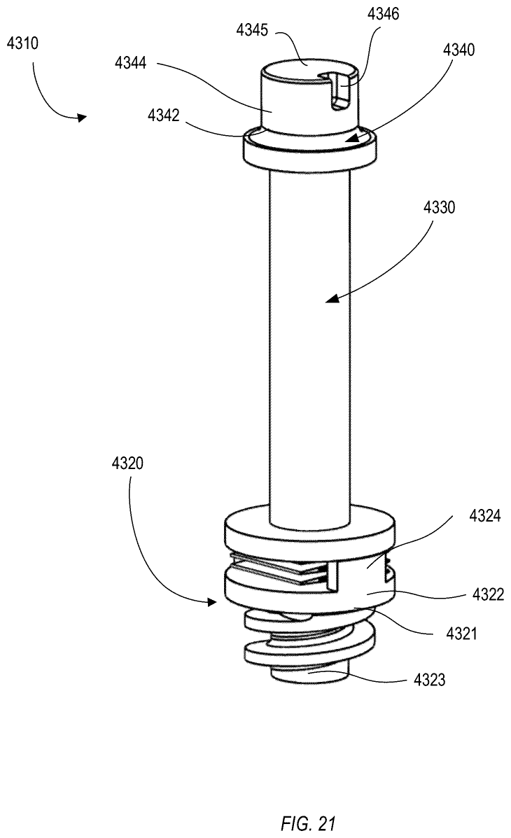

The proximal cap 4110 includes a guide wall 4115 within which the third (or proximal) member 4340 of the gas vent assembly 4310 moves. Specifically, the guide wall 4115 defines an inner cylindrical wall surface within which a guide surface 4344 of the first member 4340 (see e.g., FIGS. 13A and 21) slide during operation. The proximal cap 4110 also includes an end surface 4117 against which a portion of a delivery control mechanism (also referred to as a flow restriction assembly) rests when the medical injector 4000 is in its first configuration (i.e., the “storage” state).

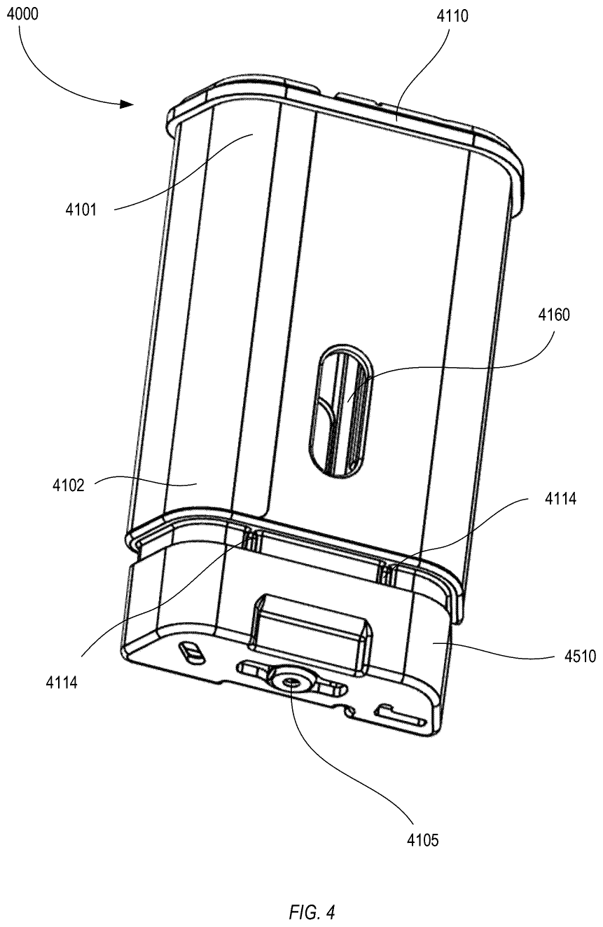

As shown in FIG. 6, the distal end portion 4102 of the housing 4100 includes a shoulder portion 4106 with a contact surface and defines a needle aperture 4105. The distal end portion 4102 also includes base rail grooves 4114 and base retention recesses 4134 (sec FIGS. 4 and 5). The shoulder portion 4106 is configured to contact a corresponding surface 4365 of the carrier body 4360 (see e.g., FIGS. 6, 18, 27 and 40) when the needle 4216 has been inserted a desired distance. In this manner the shoulder 4106 can act as an “end stop” or insertion limiting mechanism. The needle aperture 4105 is the opening through which the needle 4216 is disposed when the medical injector 4000 is actuated, as described in further detail herein.

The distal end portion 4102 of the housing also includes a release member contact surface 4126, and defines the release member aperture. As shown in FIG. 13B, the release member aperture 4145 receives a distal end portion 4552 of a release member 4550, such that the extensions 4553 of the release member 4550 engage with the release member contact surface 4126 to prevent activation of the medical injector 4000. The safety lock 4700, its components and functions are described in more detail below.

The distal base retention recesses 4134 (see FIG. 5) are configured to receive the base connection knobs 4518 of the actuator 4510 (also referred to herein as “base 4510,” sec e.g., FIGS. 25 and 26) when the base 4510 is in a first position relative to the housing 4100. The distal base retention recesses 4134 include an elongated groove extending from a proximal to distal direction to allow the base connection knobs 4518 to move and translate within the elongated groove. This allows the base retention recesses 4134 to receive the base connection knobs 4518 such that the base 4510 can move proximally relative to the housing 4100 in a first position and can move distally relative to the housing 4100 in a second position. In some embodiments, the base retention recesses 4134 can include ratcheting teeth members that engage the base connection knobs 4518 to prevent the base 4510 from moving back in the distal direction.

The base rail grooves 4114 receive the guide members 4517 of the base 4510 (sec FIGS. 4, 25 and 26). The guide members 4517 of the base 4510 and the base rail grooves 4114 of the housing 4100 engage each other in a way that allows the guide members 4517 of the base 4510 to slide in a proximal and/or distal direction within the base rail grooves 4114 while limiting lateral movement of the guide members 4517. This arrangement allows the base 4510 to move in a proximal and/or distal direction with respect to the housing 4100 but prevents the base 4510 from moving in a lateral direction with respect to the housing 4100.

FIGS. 9 and 10 provide an overview of the medicament container assembly 4200, the system actuator assembly 4500, the medicament delivery mechanism 4300, and the flow restriction assembly 4430 (which functions as a delivery control mechanism) of the medical injector 4000. Referring to FIG. 20, the medicament container assembly 4200 has a container body 4210 with a distal end portion 4213 and a proximal end portion 4212. The container body 4210 defines a volume that contains (i.e., is filled with or partially filled with) a medicament. The distal end portion 4213 of the medicament container assembly 4200 includes a neck that is coupled to the needle 4216, as described below. The proximal end portion 4212 of the medicament container assembly 4200 includes an elastomeric member 4217 (i.e., a plunger) that seals the medicament within the container body 4210. The elastomeric member 4217 is configured to move within the container body to inject the medicament from the medicament container assembly 4200.

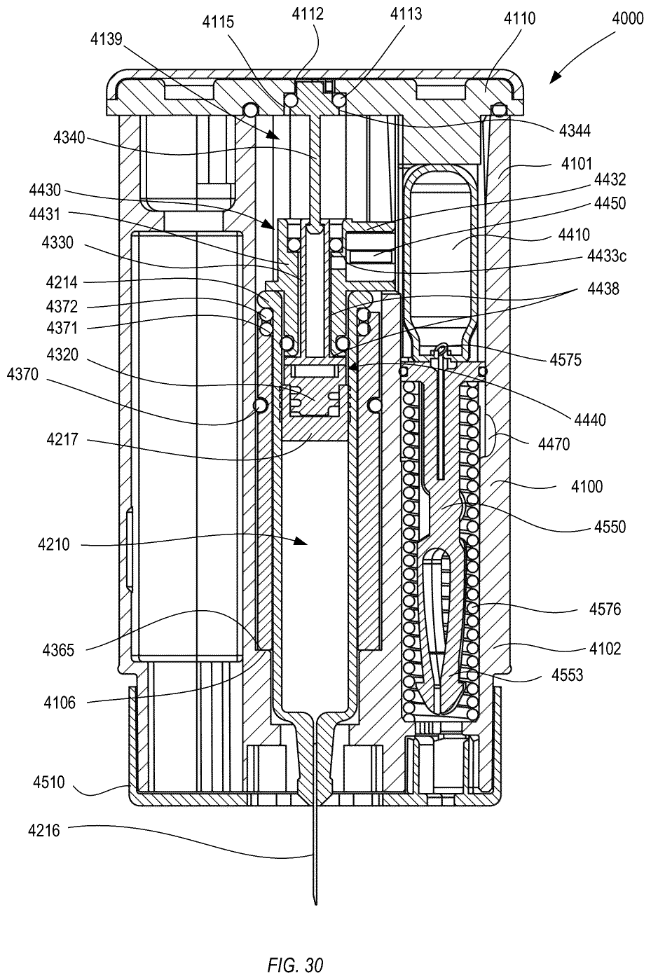

More particularly, as shown in FIG. 13A, the elastomeric member 4217 includes a proximal end portion 4218 and is coupled to the distal member 4320 of the gas venting assembly 4310. In this manner, as described below, when the pressurized gas is conveyed into the medicament cavity 4139 (or “housing gas chamber”), the pressurized gas flows through the flow restriction assembly 4430 and the gas venting assembly and into a medicament container gas chamber (i.e., a second gas chamber) located above the elastomeric member 4217 (i.e. bounded between the flow restriction assembly 4430, elastomeric member 4217 and an interior of the medicament container body 4210). The pressure within the medicament container gas chamber can have a pressure magnitude that is less than the pressure magnitude in the proximal portion of the medicament cavity 4139 (i.e., a first gas chamber). The pressure in the medicament container gas chamber exerts a force on the proximal surface 4218 to move the elastomeric member 4217 within the container body 4210 (i.e., to expel the medicament therefrom). However, as depicted in FIG. 30, because the pressure in the medicament container gas chamber is less than the pressure in the proximal portion of the medicament cavity 4139 (i.e., the first gas chamber, which acts on the carrier 4360 and/or the container body 4210), the flow restriction assembly 4430 limits movement of the elastomeric member 4217 prior to the medicament container assembly being placed in a deployed configuration (i.e., a second container position).

In so far as the elastomeric member 4217 is coupled to the gas venting assembly 4310, movement of the elastomeric member 4217 within the container body 4210 produces movement of at least a portion of the distal member 4320. Similarly stated, when the elastomeric member 4217 is exposed to a force (e.g., produced by the pressurized gas within the medicament body gas chamber 4440 acting directly on the proximal surface 4218), movement of the elastomeric member 4217 exerts a force on the distal member 4320. Specifically, distal movement of the elastomeric member 4217 can produce a tensile force on the distal member 4320.

The distal member 4320 can be coupled to the elastomeric member 4217 in any suitable manner. For example, as shown, the proximal surface 4218 receives and/or couples to a protrusion 4323 of the distal member 4320 of the gas venting assembly 4310. In some embodiments, the distal member 4320 includes a threaded portion and proximal end portion 4218 includes a corresponding threaded portion to receive the distal member 4320. In some embodiments, the threaded portion of the distal member 4320 is a self-tapping threaded portion. In other embodiments, the distal member 4320 can be threadedly coupled to the elastomeric member 4217. In yet other embodiments, the distal member 4320 can be bonded to the elastomeric member 4217 via an adhesive, a weld process, or the like

The elastomeric member 4217 can be of any design or formulation suitable for contact with the medicament. For example, the elastomeric member 4217 can be formulated to minimize any reduction in the efficacy of the medicament that may result from contact (either direct or indirect) between the elastomeric member 4217 and the medicament. For example, in some embodiments, the elastomeric member 4217 can be formulated to minimize any leaching or out-gassing of compositions that may have an undesired effect on the medicament. In other embodiments, the elastomeric member 4217 can be formulated to maintain its chemical stability, flexibility and/or sealing properties when in contact (either direct or indirect) with the medicament over a long period of time (e.g., for up to six months, one year, two years, five years or longer).

In some embodiments, the elastomeric member 4217 can be constructed from multiple different materials. For example, in some embodiments, at least a portion of the elastomeric member 4217 can be coated. Such coatings can include, for example, polydimethylsiloxane. In some embodiments, at least a portion of the elastomeric member 4217 can be coated with polydimethylsiloxane in an amount of between approximately 0.02 mg/cm² and approximately 0.80 mg/cm².

The proximal end portion 4212 of the container body 4210 includes a flange 4214 configured to be disposed within a portion of the carrier body 4360, as described in further detail herein. The flange 4214 can be of any suitable size and/or shape. Although shown as substantially circumscribing the container body 4210, in other embodiments, the flange 4214 can only partially circumscribe the container body 4210.

The medicament container assembly 4200 can have any suitable size (e.g., length and/or diameter) and can contain any suitable volume of the medicament. In some embodiments, the medicament container assembly 4200 (and any of the medicament container assemblies described herein) can be a prefilled (or prefillable) syringe, such as those manufactured by Becton Dickinson, Gerresheimer, Ompi Pharma or others. For example, in some embodiments, the medicament container assembly 4200 (and any of the medicament container assemblies described herein) can be a Becton Dickinson “BD Hypak Physiolis” prefillable syringe containing any of the medicaments described herein. The medical injector 4000 can be configured to inject any suitable dosage such as, for example, a dose of up to 4 mL of any of the medicaments described herein. In other embodiments, the medical injector 4000 can be configured to inject a dose of up to 2 mL, 3 mL, 4 mL, 5 mL, or more of any of the medicaments described herein.

The container body 4210 can be constructed from glass, and can be fitted and/or coupled to any suitable needle. For example, in some embodiments, the container body 4210 can be coupled to a needle having any suitable size. Any of the medicament container assemblies and/or prefilled syringes described herein can be coupled to a needle having a gauge size of 21 gauge, 22 gauge, 23 gauge, 24 gauge, 25 gauge, 26 gauge, 27 gauge, 28 gauge, 29 gauge, 30 gauge, or 31 gauge. Any of the medicament container assemblies and/or prefilled syringes described herein can be coupled to a needle having any suitable length, such as, for example, a length of about 0.2 inches, about 0.27 inches, about 0.38 inches, about 0.5 inches, about 0.63 inches, about 0.75 inches, or more. In some embodiments, for example, any of the medicament containers and/or prefilled syringes described herein can be coupled to a 29 gauge, needle having a length of approximately 0.5 inches.

As shown in FIG. 20, the medicament container assembly 4200 includes a needle sheath assembly 4220, that includes a sheath body 4230 and a sheath cover 4235. The needle sheath assembly 4220 includes a distal end portion 4221 and a proximal end portion 4222. The sheath body 4230 defines a bore that receives the needle 4216 and/or a distal end portion of the 4213 of the medicament container body 4210. The inner portion of the sheath body 4230 defines a friction fit with the distal end portion 4213 of the medicament container body 4210. In this manner, the needle sheath assembly 4220 can protect the user from the needle 4216 and/or can keep the needle 4216 sterile before the user actuates the medical injector 4000.

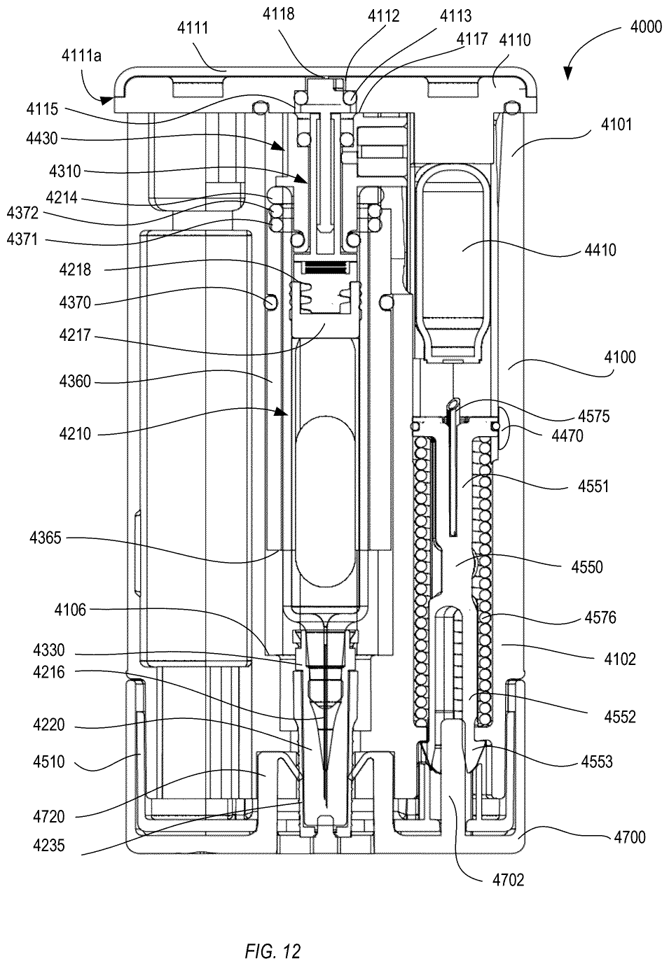

The sheath cover 4235 is disposed about (and surrounds) the sheath body 4230. The sheath cover 4235 includes a series of ribs 4236 that engage the tabs 4722 of the safety lock 4700 (see e.g., FIGS. 12, 13B, 20, 23 and 24). Specifically, the distal end portion of the sheath assembly 4220 is configured to be inserted into a space defined between the tabs 4722 of the engagement members 4721 of the safety lock 4700. The tabs 4722 are angled and/or bent towards the distal direction to allow the distal end portion of the sheath assembly 4220 to move between the engagement members 4721 in a distal direction, but not in a proximal direction. Similarly stated, the tabs 4722 include an edge that contacts the ribs 4236 of the sheath cover 4235 to prevent the safety lock 4700 from moving in a distal direction relative to the needle sheath 4220. In this manner, the needle sheath assembly 4220 is removed from the needle 4216 when the safety lock 4700 is moved in a distal direction with respect to the housing 4100.

As shown in FIG. 27, the delivery mechanism 4300 includes a gas vent assembly 4310 (also referred to as an expandable assembly), but does not rely on a piston or rigid member to move the elastomeric member 4217 within the container body 4210 to inject the medicament. Rather, the elastomeric member 4217 is moved by the force produced by the pressurized gas within the gas chamber (or medicament cavity 4139). Accordingly, the stroke length and/or the dosage amount can be set by the expanded length of the gas vent assembly 4310. In this manner, the length of the medicament container assembly 4200 and the length of the gas vent assembly 4310 can be configured such the desired dosage amount is delivered. Moreover, because the gas vent assembly 4310 moves from a collapsed to an expanded configuration, the medicament delivery mechanism 4300 can fit within the same housing 4100 regardless of the fill volume, the delivery volume and/or the ratio of the fill volume to the delivery volume. In this manner, the same housing and production tooling can be used to produce devices having various dosages of the medicament. For example, in a first embodiment (e.g., having a fill volume to delivery volume ratio of 0.4), the medicament container has a first length and the second movable member has a first length. In a second embodiment (e.g., having a fill volume to delivery volume ratio of 0.6), the medicament container has a second length shorter than the first length, and the second movable member has a second length longer than the first length. In this manner, the stroke of the device of the second embodiment is longer than that of the device of the first embodiment, thereby allowing a greater dosage. The medicament container of the device of the second embodiment, however, is shorter than the medicament container of the device of the first embodiment, thereby allowing the components of both embodiments to be disposed within the same housing and/or a housing having the same length.

In some embodiments, the medical injector 4000 is configured such that a ratio of the housing length L_{H} to the container length L_{C} (which includes the needle extending from the end of the container body) is less than about 1.5. In other embodiments, the medical injector 4000 is configured such that a ratio of the housing length L_{H} to the container length L_{C} is less than about 1.25. In yet other embodiments, the medical injector 4000 is configured such that a ratio of the housing length L_{H} to the container length L_{C} is less than about 1.1.

In some embodiments, the medical injector 4000 is configured such that a ratio of the housing length L_{H} to a sum of the container length L_{C}, the carrier distance, and the stroke is less than about 1.1. In other embodiments, the medical injector 4000 is configured such that a ratio of the housing length L_{H} to a sum of the container length L_{C}, the carrier distance, and the stroke is less than about 1.0. In yet other embodiments, the medical injector 4000 is configured such that a ratio of the housing length L_{H} to a sum of the container length L_{C}, the carrier distance, and the stroke is less than about 0.9.

As shown in FIGS. 9, 10, and 31, the system actuator assembly 4500 includes the base 4510, a release member 4550 and a spring 4576. FIG. 10 shows certain internal components of the medical injector 4000 without the base 4510 and the safety lock 4700 so that the release member 4550 can be more clearly shown. The release member 4550 has a proximal end portion 4551 and a distal end portion 4552, and is movably disposed within the distal end portion of the gas container cavity 4151. The proximal end portion of the release member 4550 includes a sealing member 4574 and a puncturer 4575. The sealing member 4574 is configured to engage the sidewall of the housing 4100 defining the gas container cavity 4151 such that the proximal end portion of the gas container cavity 4151 is fluidically isolated from the distal end portion of the gas container cavity 4151. In this manner, when gas is released from the gas container 4410, the gas contained in the proximal end portion of the gas container cavity 4151 is unable to enter the distal end portion of the gas container cavity 4151. The puncturer 4575 of the release member 4550 is configured to contact and puncture a frangible seal 4413 on the gas container 4410 when the release member 4550 moves proximally within the gas container cavity 4151.

The distal end portion 4552 of the release member 4550 includes extensions 4553. The extensions 4553 have projections that include tapered surfaces and engagement surfaces. Further, the extensions 4553 define an opening between the adjacent extensions 4553. The engagement surfaces are configured to extend through the release member aperture and contact the release member contact surface of the housing 4100, as shown in FIG. 28. In this manner, the engagement surfaces limit proximal movement of the release member 4550.

The opening defined by the extensions 4553 is configured to receive the safety lock protrusion 4702 of the safety lock 4700 (see e.g., FIGS. 12 and 13B) when the safety lock 4700 is coupled to the housing 4100 and/or the base 4510. The safety lock protrusion 4702 is configured to prevent the extensions 4553 from moving closer to each other. Said another way, the safety lock protrusion 4702 is configured to ensure that the extensions 4553 remain spaced apart and the engagement surfaces remain in contact with the release member contact surface of the housing 4100. In some embodiments, for example, the release member 4550 and/or the extensions 4553 can be constructed from any suitable material configured to withstand deformation that may occur when exposed to a load over an extended period of time.

The tapered surfaces of the extensions 4553 are configured to contact corresponding tapered surfaces 4557 of the base 4510 when the base 4510 is moved proximally relative to the housing 4100. Accordingly, when the base 4510 is moved proximally relative to the housing 4100, the extensions 4553 are moved together by the tapered surfaces. The inward movement of the extensions 4553 causes the release member 4550 to disengage the release member contact surface 4126 of the housing 4100, thereby allowing the release member 4550 to be moved proximally along its longitudinal axis as the spring 4576 expands (see FIG. 31).

The gas container 4410 includes a distal end portion 4411 and a proximal end portion 4412, and is configured to contain and/or produce a pressurized gas. The distal end portion 4411 of the gas container 4410 contains a frangible seal 4413 configured to break when the puncturer 4575 of the release member 4550 contacts the frangible seal 4413. The gas container retention member 4180 of the proximal cap 4110 of the housing 4100 is configured to receive and/or retain the proximal end portion 4412 of the gas container 4410. Said another way, the position of the gas container 4410 within the gas container cavity 4151 is maintained by the gas container retention member 4180. As shown in FIGS. 9 and 10, the length of the gas container retention member 4180 and the length of the release member 4550 collectively determine the distance between the puncturer 4575 and the frangible seal 4413 when the medical injector 4000 is in the storage configuration. Accordingly, this distance, which is the distance through which the puncturer 4575 travels when the medical injector 4000 is actuated, can be adjusted by changing the length of the gas container retention member 4180 and/or the length of the release member 4550. In some embodiments, the actuation time and/or the force exerted by the puncturer 4575 on the frangible seal 4413 can be adjusted by changing the distance between the puncturer 4575 and the frangible seal 4413.

As shown in generally in FIG. 9, the medicament delivery mechanism 4300 includes a carrier assembly 4390, a flow restriction assembly 4430 (also referred to as a delivery control mechanism), and a gas vent assembly 4310. The carrier assembly 4390 and the gas vent assembly 4310 are each movably disposed within the medicament cavity 4139 of the housing 4100. For example, the carrier assembly 4390, with the medicament container assembly 4200, is configured to move between a withdrawn position (i.e., a first position as depicted in FIG. 12) and a deployed position (i.e., a second position as depicted in FIG. FIG. 30) in response to a force exerted by the pressurized gas in the proximal portion of the medicament cavity 4139. As shown in FIGS. 16-20, the carrier assembly 4390 includes a carrier body 4360 and a retraction spring 4380. The carrier body 4360 includes a distal end portion 4361 and a proximal end portion 4362. The proximal end portion 4362 of the carrier body 4360 defines an opening within which the medicament container body 4210 is disposed. The proximal end portion 4362 also includes a proximal surface 4376, forms a portion of the boundary of the housing gas chamber (i.e., the portion of the medicament cavity 4139 within which the pressurized gas flows in a first phase of expansion when the medicament container body 4210 is actuated within the housing 4100). In this manner, the pressurized gas produces a force on the proximal surface 4376, which moves the carrier assembly 4390 distally within the housing 4100.

An inner surface of the proximal end portion 4362 defines a groove within which a first O-ring 4371 and a second O-ring 4372 are disposed. The first O-ring 4371 and the second O-ring 4372 are disposed between a top surface of the carrier body 4360 and the flange 4214 of the medicament container body 4210. In this manner, the first O-ring 4371 and the second O-ring 4372 form a substantially fluid-tight seal. Accordingly, when pressurized gas flows into the proximal portion of the medicament cavity 4139 (i.e., the housing gas chamber), the area between the inner surface of the carrier body 4360 and the medicament container body 4210 is sealed. The first O-ring 4371 and the second O-ring 4372 also dampen any impact on the flange 4214.

An outer surface of the carrier body 4360 defines an O-ring groove and includes an outer O-ring 4370. The outer surface is configured to slide against sidewalls 4139a within the medicament cavity 4139 (see FIG. 6), and the O-ring 4370 and an inner surface of the housing 4100 define a form a substantially fluid-tight seal. Accordingly, when pressurized gas flows into the proximal portion of the medicament cavity 4139, the area between the outer surface of the carrier body 4360 and the inner surface of the housing 4100 is sealed. The outer O-ring 4370 is in a fixed location relative to each of the inner O-rings 4371, 4372. In other embodiments, however, a carrier assembly can include components that move relative to each other such that an outer seal member moves relative to an inner seal member.

The distal end portion 4361 of the carrier body 4360 has an open end. Thus, as shown in FIGS. 16-19, the distal end portion 4213 of the medicament container body 4210 extends beyond the carrier body 4360. Additionally, the distal end portion 4361 of the carrier body 4360 includes two extensions (or “legs”) that collectively define an opening 4375. This opening is configured to align with the status apertures 4130, 4160 of the housing to allow viewing of the medicament within the medicament container assembly, the elastomeric member 4217 or the like. The distal end portion 4361 also includes an end surface 4365 configured to contact the shoulder portion 4106 of the housing 4100 (see e.g., FIG. 30) when the needle 4216 has been inserted a desired distance.

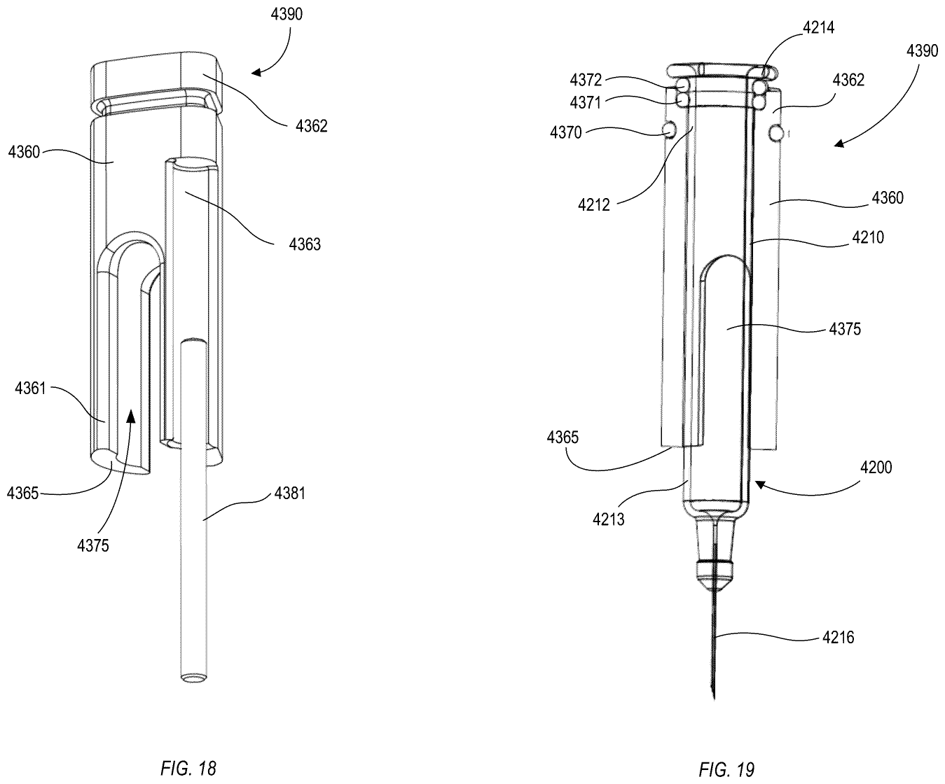

The retraction spring 4380 is disposed within a spring pocket 4363 defined by the outer surface of the carrier body 4360, as shown in FIG. 18. The retraction spring 4380 is disposed about a spring pin 4381 that limits buckling or other lateral movement of the retraction spring 4380 during use. The delivery control mechanism 4430 (also referred to as the flow restriction assembly) is configured to regulate the pressure applied on the elastomeric member 4217 to control the rate in which the elastomeric member 4217 moves within the medicament container body 4210. In this manner, the flow rate at which the medicament is dispensed out of the medicament container body 4210 via the needle 4216 as the elastomeric member 4217 moves through its travel stroke can be controlled. Control of the flow rate of medicament leaving the device can minimize pain or discomfort, particularly when the medicament is highly viscous (e.g., greater than about 100 centipoise at room temperature). Additionally, where the medicament includes high molecule weight compounds (e.g., greater than about 5 kDa), reduced injection force less than a force required to overcome the retraction spring 4380 prevents shearing and therefore damage to the medicament or therapeutic substance.

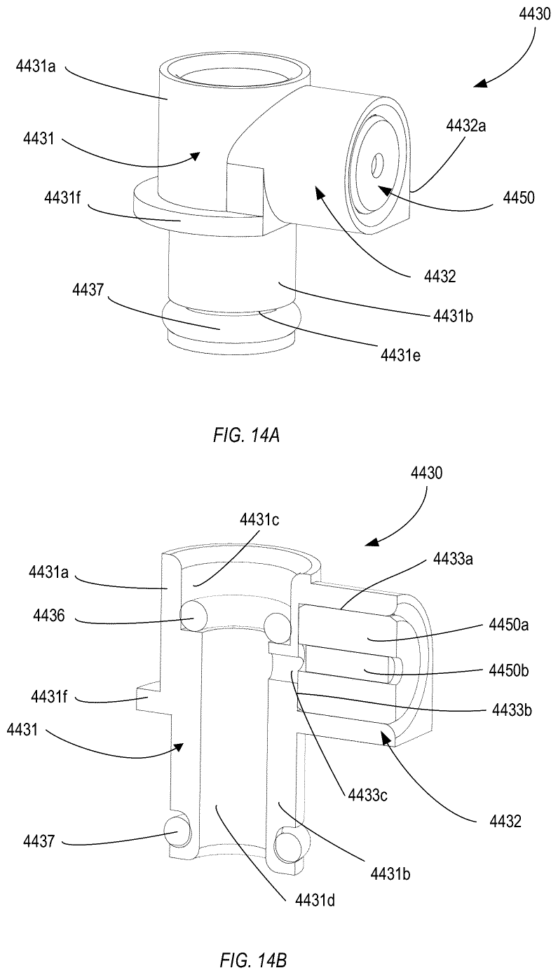

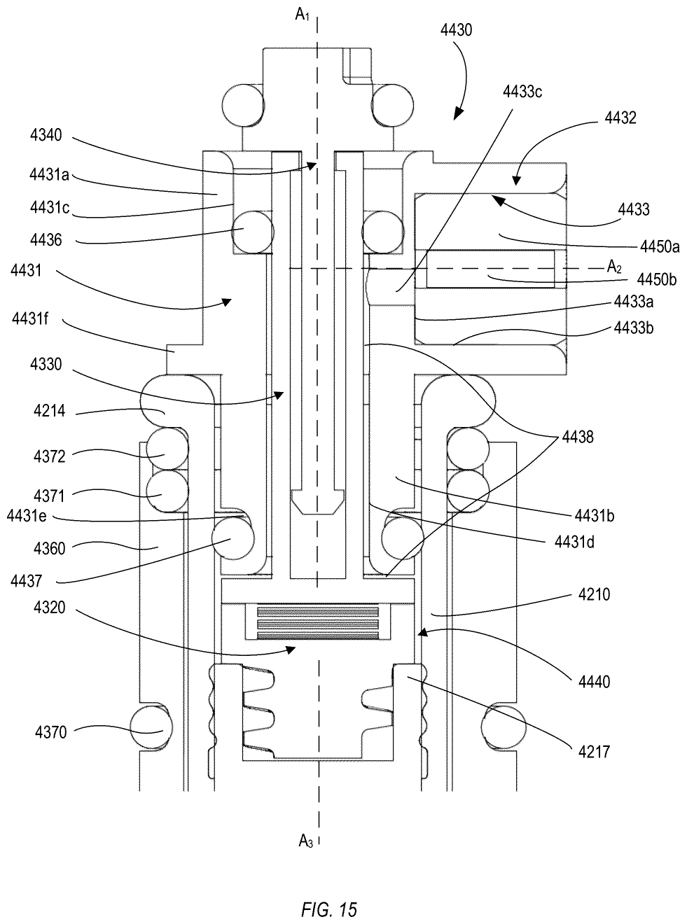

As shown in FIGS. 14A, 14B, and 15, the delivery control mechanism 4430 includes a first body portion 4431 and a second body portion 4432. The second body portion 4432 extends from the first body portion 4431. The second body portion 4432 includes a flow restriction retainer 4433 configured to support at least a portion of a flow restriction member 4450. As shown in FIG. 14B, the flow restriction retainer 4433 includes a cylindrical inner surface 4433a and an end surface 4433b. The end surface 4433b defines a through-hole 4433c extending into an interior portion of the first body portion 4431. In this manner, the interior of the first body portion 4431 is in fluid communication with the second body portion 4432. Although the through-hole 4433c is shown as being non-coaxial with a center of the flow restriction member 4450, in some embodiments, the through-hole 4433c is co-axial with the flow restriction member 4450. In some embodiments, at least a portion of a flow restriction element 4450b of the flow restriction member 4450 overlaps with a portion through-hole 4433c. In some embodiments, at least 50% of the flow restriction element 4450b overlaps with the through-hole 4433c. In some embodiments, the flow restriction member 4450 is press fit or threaded into the flow restriction retainer 4433.

The flow restriction member 4450 includes a sleeve member 4450a and a flow restriction element 4450b, and the flow restriction element 4450b is supported within the sleeve member 4450a. In some embodiments, the sleeve member 4450a is a metal sleeve. In some embodiments, the metal sleeve is made of stainless steel or brass. In some embodiments, the flow restriction element 4450b is a porous material. In some embodiments, the porous material is sintered porous metal. In some embodiments, the flow restriction element 4450b is calibrated with nitrogen gas (N₂) at 30 psig (inlet side) to atmosphere (outlet side) at standard temperature and pressure to have a flow rate of between 0.5 to 3 standard cubic centimeter per minute (sccm). In some embodiments, the flow restriction element 4450b is calibrated with nitrogen gas (N₂) at 30 psig (inlet side) to atmosphere (outlet side) at standard temperature and pressure to have a flow rate of between about 0.75 and 1.5 standard cubic centimeter per minute (sccm). In some embodiments, the flow restriction element 4450b is calibrated with nitrogen gas (N₂) at 30 psig (inlet side) to atmosphere (outlet side) at standard temperature and pressure to have a flow rate of about 1 standard cubic centimeter per minute (sccm). As described herein, standard temperature is 60ºF (15.6°) C. and standard pressure is 14.696 psia (101.3 kPa).

In some embodiments, the compressed gas supplied by the gas container 4410 is an argon gas and the flow restriction element 4450b has a flow rate rating of about 0.75 and 1.5 sccm based on the nitrogen gas calibration described above. In some embodiments, the compressed gas supplied by the gas container 4410 is an argon gas and the flow restriction element 4450b has a flow rate rating of about 1 sccm based on the nitrogen gas calibration described above. In some embodiments, the compressed gas in the gas container 4410 has a molecular weight greater than the molecular weight of argon. For example, in some embodiments, the compressed gas supplied by the gas container 4410 is R134a (Tetrafluoroethane) and the flow restriction element 4450b has a flow rate rating of about 10 to 100 sccm based on the nitrogen gas calibration described above. In some embodiments, the compressed gas supplied by the gas container 4410 is R134a (Tetrafluoroethane) and the flow restriction element 4450b has a flow rate rating of about 20 to 40 sccm based on the nitrogen gas calibration described above.

In some embodiments, the flow rate of the medicament can be reduced to less than 0.2 mL/sec (or in some embodiments between 0.05 mL/sec and 0.01 mL/sec) using gas pressure that is initially supplied to medicament cavity 4139 and through the flow restriction member 4450. The lower injection forces and/or slower delivery (compared with pressures supplied directly from the medicament cavity 4139 to the elastomeric member) can produce laminar flow of the medicament through the needle, prevent shearing of high molecular weight compounds in the medicament, and/or reduce pain sensed by a patient particularly if the medicament being delivered is very high viscosity (e.g., greater than about 100 centipoise at room temperature). In some embodiments, a screen or mesh protective member can be provided on a proximal side of the flow restriction member 4450 to prevent any particulate or debris from clogging the flow restriction element 4450b during operation.

The first body portion 4431 includes a proximal end portion 4431a and a distal end portion 4431b. The proximal end portion 4431a includes a first inner cylindrical surface 4431c and the first inner cylindrical surface 4431c is configured to support an O-ring 4436, which in turn contacts a portion of the gas vent assembly 4310 to seal an interior of the first body portion 4431 from housing gas chamber portion of the medicament cavity 4139 as described in further detail herein.

The distal end portion 4431b extends into a proximal end portion of the medicament container body 4210. The distal end portion 4431b also includes a second inner cylindrical surface 4431d and an outer cylindrical groove 4431e configured to support an O-ring 4437, which in turn contacts and seals against an interior wall of the medicament container body 4210. The first inner cylindrical surface 4431c and the second inner cylindrical surface 4431d define a bore extending from the proximal end portion 4431a to the distal end portion 4431b. The bore permits the gas vent assembly 4310 to extend into and through the first body portion 4431. A portion of the bore further defines a gas passageway between the second body portion 4432 and a medicament body gas chamber 4440 as described in detail below. As shown in FIG. 14B, an inner diameter of the first inner cylindrical surface 4431c is larger than the inner diameter of the second inner cylindrical surface 4431d. The first body portion 4431 further includes a flange portion 4431f extending radially from an outer surface of the first body portion 4431. The flange portion 4431f is configured to mount onto the flange 4214 of the medicament container body 4210 or onto a proximal end portion of the carrier 4360.

As shown in FIG. 15, the first body portion 4431 defines a first axis A₁, the second body portion 4432 defines a second axis A₂, and the first axis is non-parallel with the second axis. In some embodiments, the first axis A₁ and the second axis A₂ are perpendicular to one another. In other embodiments, the first axis and the second axis define an acute angle therebetween. In some embodiments, the medicament container body 4210 defines a third axis A₃, and the first axis of the first body portion 4431 is parallel with the third axis A₃. In some embodiments, the second body portion 4432 includes a guide surface 4432a (see FIG. 14B) to contact a wall or guide member 4135a of the housing 4100 (see FIG. 6) to prevent rotation of the delivery control mechanism 4430 about the first axis A₁ during operation.

The gas vent assembly 4310 is configured to expand and/or change configurations during operation of the medical injector 4000, and selectively produces a pathway through which pressurized gas escapes the medicament cavity 4139 after delivery of the medicament. By releasing or removing the force from the carrier body 4360, the delivery control mechanism 4430 and/or the medicament container assembly 4200, the retraction spring 4380 can move the carrier body 4360 proximally to retract the needle 4216. Notably, the gas vent assembly 4310 does not exert a distal force on the elastomeric member 4217, but rather, is carried distally by the elastomeric member 4217 during delivery of the medicament. Thus, this arrangement is considered a “pistonless” delivery system, because the force for insertion and medicament delivery is provided via the pressurized gas acting either directly upon the medicament container assembly 4200 (e.g., the proximal surface 4218 of the elastomeric member 4217), the delivery control mechanism 4430 (e.g., the first body portion 4431 and the second body portion 4432 of the delivery control mechanism extending out of the medicament container body 4210), and/or the carrier assembly 4390 (e.g., the proximal surface 4376 of the carrier body 4360), or indirectly through gas pressure supplied from the medicament cavity 4139 through the delivery control mechanism 4430 via the flow restriction member as described herein.

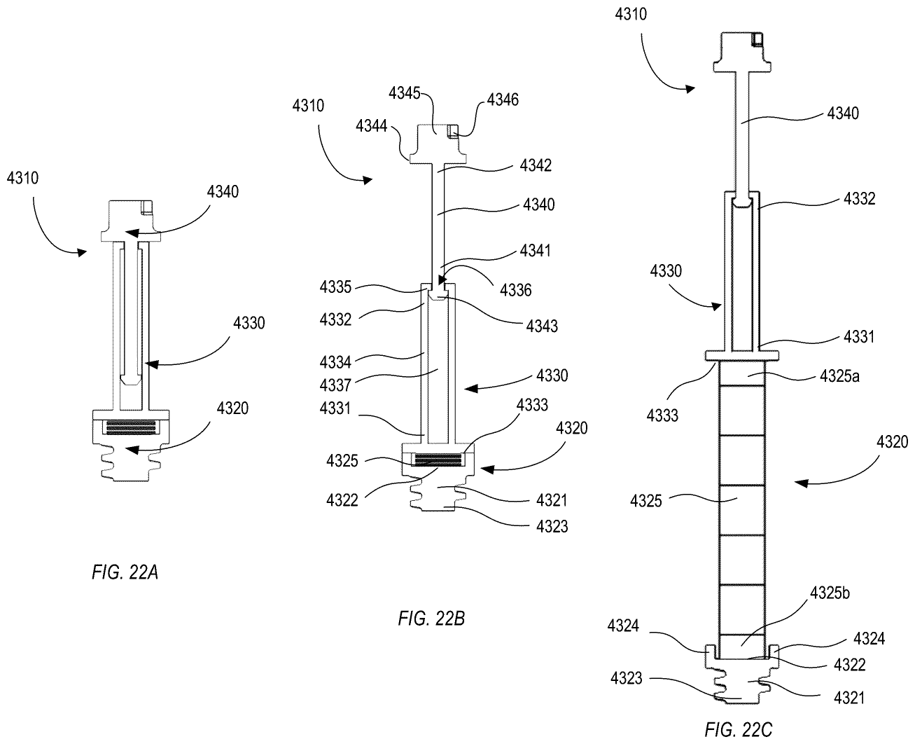

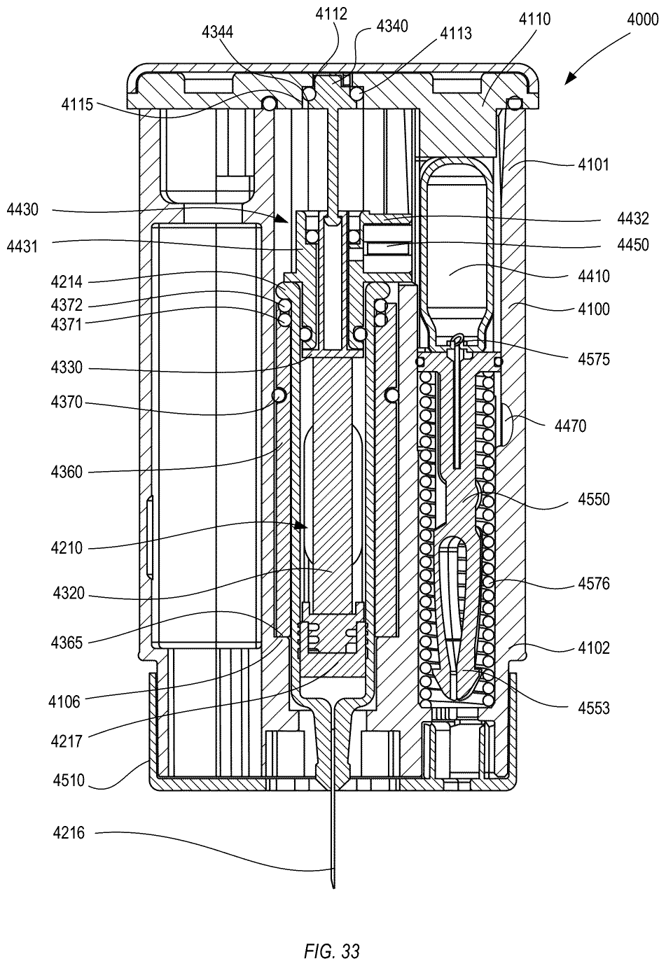

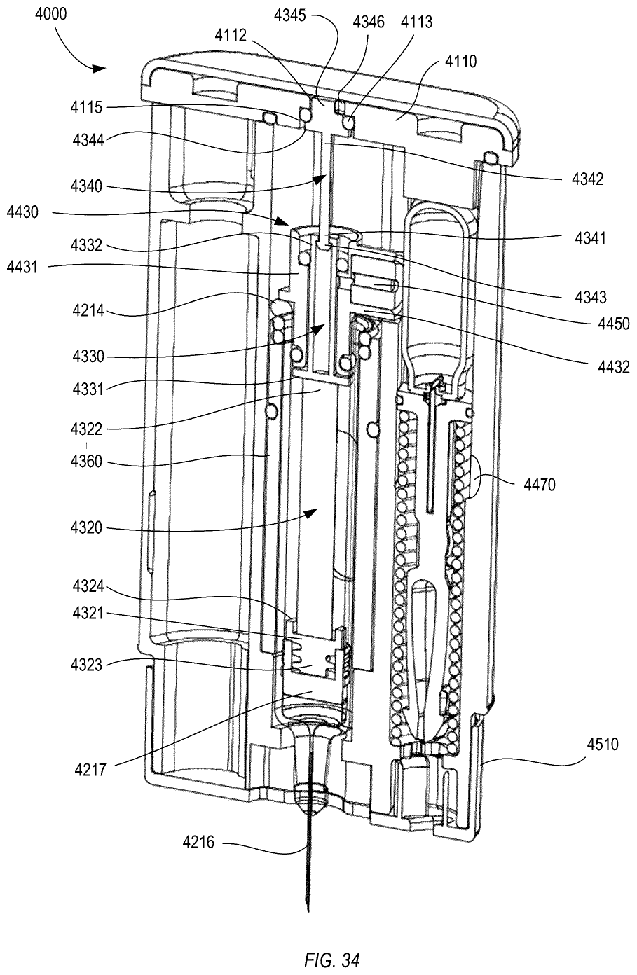

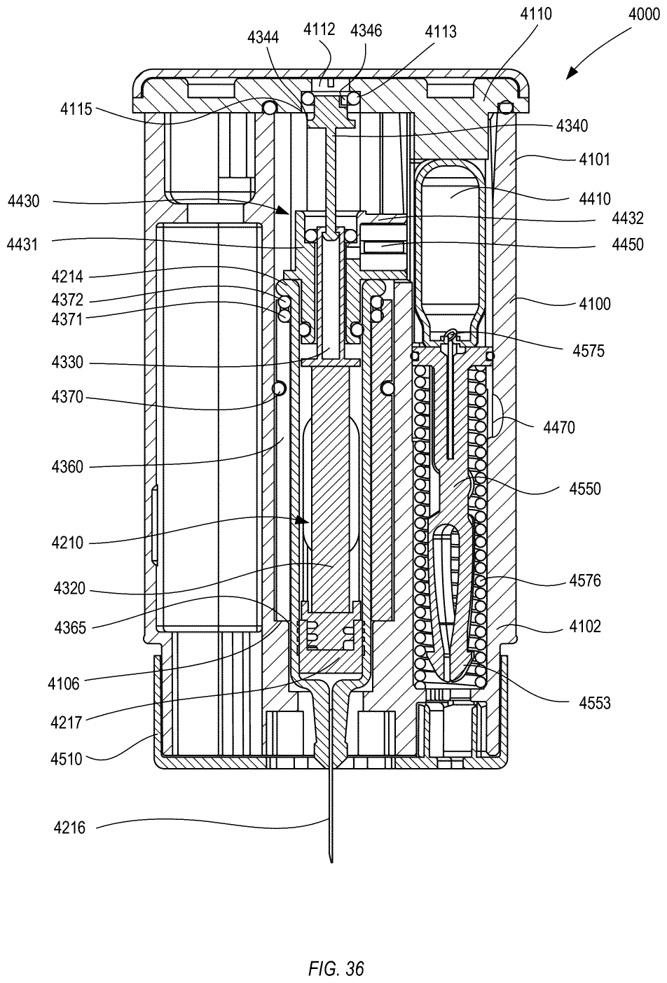

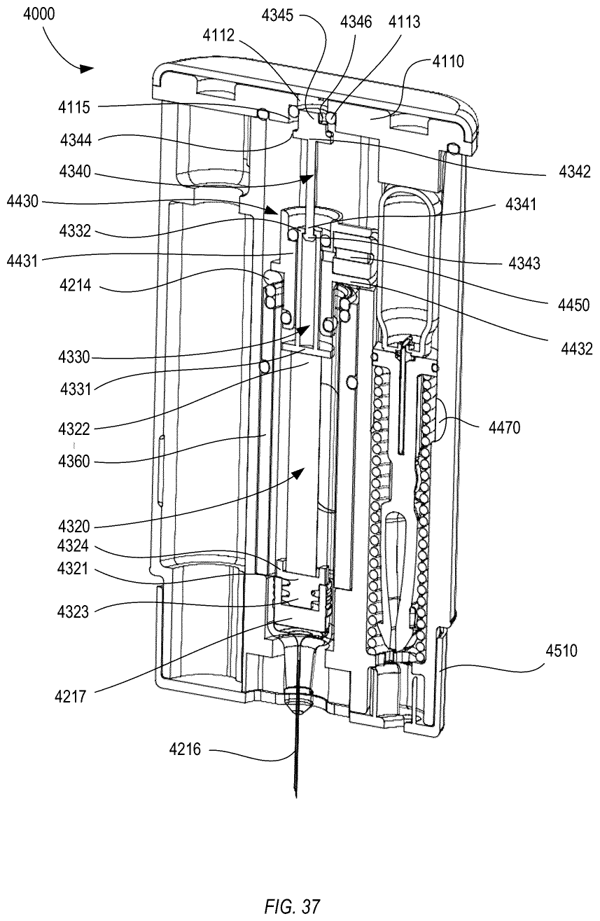

As shown in FIGS. 21, 22A, 22B, and 22C, the gas vent assembly 4310 includes a first (or distal) member 4320, a second (or central) member 4330 and a third (or proximal) member 4340. These components are nested together such that the gas vent assembly 4310 can be transitioned from a collapsed configuration (FIGS. 12, 13A and 28) to an expanded configuration (FIGS. 33 and 34, just prior to complete delivery of medicament), and a series of partially expanded configurations therebetween (see e.g., FIG. 30). The gas vent assembly 4310 reaches the expanded configuration just prior to a complete dose of medicament being delivered. Once the gas vent assembly 4310 has been placed in the expanded configuration, the elastomeric member 4217 continues to travel a final distance to deliver the remaining amount from the complete dose, which in turn pulls on the valve portion 4345 to at least partially unseat it from the opening 4112. Stated in a different manner, the length of the gas vent assembly 4310 in the expanded configuration is selected to expand and reach the expanded configuration before the end of travel of the elastomeric member 4217 continues. When the gas vent assembly 4310 is in the expanded configuration and continues to travel with the elastomeric member 4217 the final distance to finish delivery of the complete dose (FIGS. 36 and 37, after delivery of the medicament is complete), the opening 4112, the O-ring 4113 and the passageway 4346 collectively allow the pressurized gas from the housing gas chamber of the medicament cavity 4139 to escape the medicament cavity 4139, such that needle retraction can occur.

The first member 4320 includes a proximal end portion 4322 and a distal end portion 4321. The distal end portion 4321 includes a protrusion 4323 configured to matingly engage the elastomeric member 4217. In this manner, movement of the elastomeric member 4217 distally causes movement of first member 4320 distally. In some embodiments, the protrusion 4323 is a threaded portion that matingly engages the elastomeric member 4217. The proximal end portion 4322 includes a pair of retention walls 4324 configured to engage a corresponding distal end surface 4333 of the second (or central) member 4330. In some embodiments, the pair of retention walls 4324 each include a pin or tab that loosely couples the proximal end portion 4322 of the first member 4320 to the distal end surface 4333 of the second member 4330 to assist in assembly of the device, but separates once pressure is applied on the elastomeric member 4217 in a distal direction. The proximal end portion 4322 further includes a flexible expansion member 4325 that is fixed at a first end 4325a to the proximal end portion 4322 of the first member 4320 and is fixed at a second end 4325b to the distal end surface 4333 of the second member 4330. During the second phase of expansion (i.e., movement of the elastomeric member 4217 as the device transitions from the fourth configuration to the fifth configuration) the elastomeric member 4217 is moved distally within the medicament container body 4210. As the elastomeric member 4217 is moved, the first end 4325a and the second end 4325b move away from each other as the flexible expansion member 4325 expands. As shown in FIGS. 21 and 22A-22C, the flexible expansion member 4325 is collapsed down with an accordion fold. In some embodiments, the flexible expansion member 4325 is a filament or a band. In some embodiments, the flexible expansion member 4325 is over-molded with a plastic material. In some embodiments, the flexible expansion member 4325 is a cable that is initially coiled or spooled between the proximal end portion 4322 of the first member 4320 and the distal end surface 4333 of the second member 4330.

The second member 4330 includes a distal end portion 4331 and a proximal end portion 4332 The distal end portion 4331 includes the distal end surface 4333 that engages the first member 4320. The second member 4330 includes a sidewall 4334 extending from the distal end portion 4331 to the proximal end portion 4332. The proximal end portion 4332 includes a shoulder portion 4335 extending towards a center of the second member 4330 and the shoulder portion 4335 defines an opening 4336. The distal end portion 4331, the proximal end portion 4332, and the sidewall 4334 define an internal volume 4337.

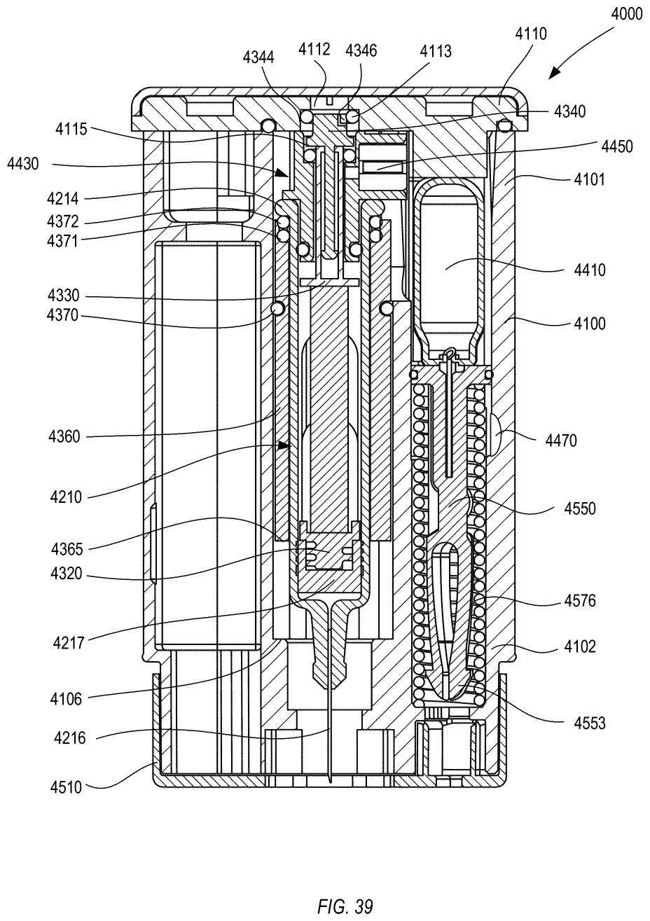

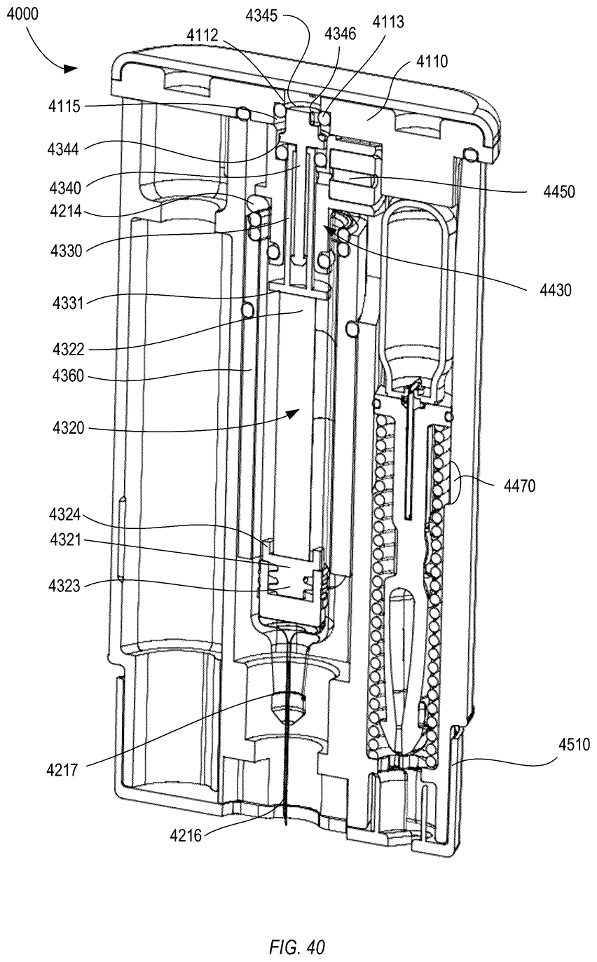

The third member 4340 includes a distal end portion 4341 and a proximal end portion 4342. The distal end portion 4341 extends through opening 4336 of the second member 4330. The distal end portion 4341 includes a distal protrusion 4343 configured to travel within the internal volume 4337 and to engage the shoulder portion 4335 of the second member 4320 when the second member 4320 is extended away from the third member 4340. In this manner, distal protrusion 4343 limits movement of the second member 4340 as it extends away from the third member 4340 during the first phase of expansion as described herein. The proximal end portion 4342 includes a guide surface 4344 and a valve portion 4345. The guide surface 4344 engages the O-ring 4113 and slides within the guide wall 4115 of the proximal cap 4110 (FIG. 13A). The valve portion 4345 defines a passageway 4346 that bypasses the O-ring 4113 when the valve portion 4345 is placed in an open configuration (see FIGS. 39 and 40). As shown in FIG. 21, the passageway 4346 is a recessed portion extending into the proximal end portion 4342.

In some embodiments, the gas vent assembly 4310 is configured (e.g., the vent opening 4112 is sized and/or shaped) to maintain the force produced by the compressed gas on the medicament container assembly 4200 at a magnitude that is greater than the force applied by the retraction spring 4380 during a time to deliver the final portion of the dose. In other words, the gas vent assembly 4310 is tuned such that the force of the compressed gas within the proximal portion of the medicament cavity 4139 (e.g., the gas chamber) remains greater than the retraction force during the period of time required to complete the delivery of the final portion of the dose. In some embodiments, the venting duration commences with the actuating of a valve member 4345 of the gas vent assembly 4310 and may culminate with the delivery of the final portion of the dose. It should be appreciated that the venting duration may correspond to the desired time to deliver the final portion of the dose and may, therefore, be determined based on the delivery properties of the composition. It should further be appreciated that maintaining the force of the compressed gas at a magnitude that is greater than the retraction force beyond the completion of the delivery of the final portion may result in the undesirable maintaining of the needle in the body of patient for longer than is required to deliver the dose. However, the venting the compressed gas too quickly may be undesirable in that it may permit the force of the compressed gas to fall below the retraction force prior to the final portion being completely delivered. In addition to the timing of when the venting starts, the rate of venting can also impact the timing and accuracy of delivery of the final portion of the dose.

In some embodiments, the gas vent assembly 4310, including the vent opening size and/or movement of a valve body within the opening, and/or the instant at which the venting is initiated can be configured so that the force produced by the compressed gas within the proximal portion of the medicament cavity 4139 decreases to a magnitude that is less than the force applied by the retraction spring 4380 concurrent with the conclusion of the delivery of the final portion of the dose. For example, the gas vent assembly 4310 is configured (e.g., tuned) by selecting the vent opening size, the vent opening shape, the valve body size, and/or the valve body shape to achieve a desired rate of decrease of the force within the internal volume. The desired rate of decrease of the force may, for example, be based on the desired delivery properties of the composition, characteristics of pressurized gas, and/or environmental parameters. For example, in some embodiments, the vent opening is configured (e.g., sized) to establish a venting duration of at least 0.5 seconds and less than 1.5 seconds. In some additional embodiments, the vent opening is sized to establish a venting duration of at least 5 seconds and less than or equal to 15 seconds.

In some embodiments, the initial pressure within the proximal portion of the medicament cavity 4139 is about 100 psi. The final pressure in the proximal portion of the medicament cavity 4139 after the entire medicament (e.g., 10 mL) has been delivered is generally above 80 psi. Additionally, the average elapsed time for delivering a first 20% of the medicament is about 17 seconds, the average elapsed time for delivering a second 20% of the medicament is about 19 seconds, the average elapsed time for delivering a third 20% of the medicament is about 21 seconds, the average elapsed time for delivering a fourth 20% of the medicament is 25 seconds, the average elapsed time for delivering a fifth and final 20% of the medicament is about 31 seconds. The flow restriction assembly 4430 and/or gas vent assembly 4310 is configured to limit and moderate delivery of the medicament such that the first 20% of the delivered volume is not delivered more than two times faster than the last 20% of the delivered volume. In other words, the first 20% of the delivered volume is delivered less than two times faster than the last 20% of the delivered volume (i.e., about 17 seconds versus about 31 seconds). In some embodiments, the elapsed time to dispense the last 20% of the delivered volume is between about one to two times the elapsed time to dispense the first 20% of the delivered volume. For example, if the elapsed time to dispense the first 20% of the delivered volume is about 15 seconds, the elapsed time to dispense the last 20% of the delivered volume is between about 15 seconds to about 30 seconds. In some embodiments, the elapsed time to dispense the last 20% of the delivered volume is between about 100% to about 195% of the elapsed time to dispense the first 20% of the delivered volume.

As shown in FIGS. 12, 23, and 24, the safety lock 4700 includes a safety lock protrusion 4702 and an engagement portion 4720. As described above, when the safety lock 4700 is in a first (locked) position, the safety lock protrusion 4702 is configured to be disposed in the opening defined by the extensions 4553 of the release member 4550. Accordingly, the safety lock protrusion 4702 is configured to prevent the extensions 4553 from moving closer to each other, thereby preventing proximal movement of the release member 4550 and/or delivery of the medicament.

The engagement portion 4720 of the safety lock 4700 includes engagement members 4721 that extend in a proximal direction. The engagement members 4721 have tabs 4722 that extend from a surface of the engagement members. The tabs 4722 engage the ribs 4236 of the sheath cover 4235 to limit relative movement between the safety lock 4700 and the needle sheath assembly 4220, as described above. In this manner, the needle sheath assembly 4220 can protect the user from the needle 4216 and/or can keep the needle 4216 sterile before the user actuates the medical injector 4000, and the needle sheath assembly 4220 can be removed from about the needle 4216 when the safety lock 4700 is removed.

The outer surface of the safety lock 4700 include a grip portion (recessed finger grips) and indicia thereon. The recessed finger grips provides an area for the user to grip and/or remove the safety lock 4700 from about the housing 4100. The indicia provide instruction on how to remove the safety lock 4700. In some embodiments, for example, indicia can indicate the direction the user should pull the safety lock 4700 to remove the safety lock 4700.

FIGS. 25 and 26 show the base (or actuator) 4510 of the medical injector 4000. The base 4510 includes a proximal (or inner) surface 4511, a distal (or outer) surface 4523 and base connection knobs 4518. The distal surface 4523 is disposed against a target surface (not shown) during use of the injector 4000. As described below, the housing 4100 is moved distally relative to the base 4510 and/or the distal surface 4523, thereby causing the base 4510 to move proximally relative to the housing 4100 to actuate the medical injector 4000. The base 4510 defines a needle aperture 4513 and a safety lock protrusion aperture 4514. The needle aperture 4513 is configured to receive the needle 4216 when the medical injector 4000 is actuated. The safety lock protrusion aperture 4514 of the base 4510 receives the safety lock protrusion 4702 of the safety lock 4700 when the safety lock 4700 is coupled to the housing 4100 and/or the base 4510.

The proximal surface 4511 of the base 4510 includes guide members 4517 and protrusions 4515. The guide members 4517 of the base 4510 engage and/or slide within the base rail grooves 4114 of the housing 4100, as described above. The protrusions 4515 of the base 4510 engage the tapered surfaces of the extensions 4553 of the release member 4550. As described in further detail herein, when the safety lock 4700 is removed and the base 4510 is moved in a proximal direction with respect to the housing 4100, the protrusions 4515 of the base 4510 are configured to move the extensions 4553 of the release member 4550 closer to each other, actuating the medicament delivery mechanism 4300. In some embodiments, the base connection knobs 4518 engage the base retention recesses 4134 in a way that allows proximal movement of the base 4510 but limits distal movement of the base 4510.

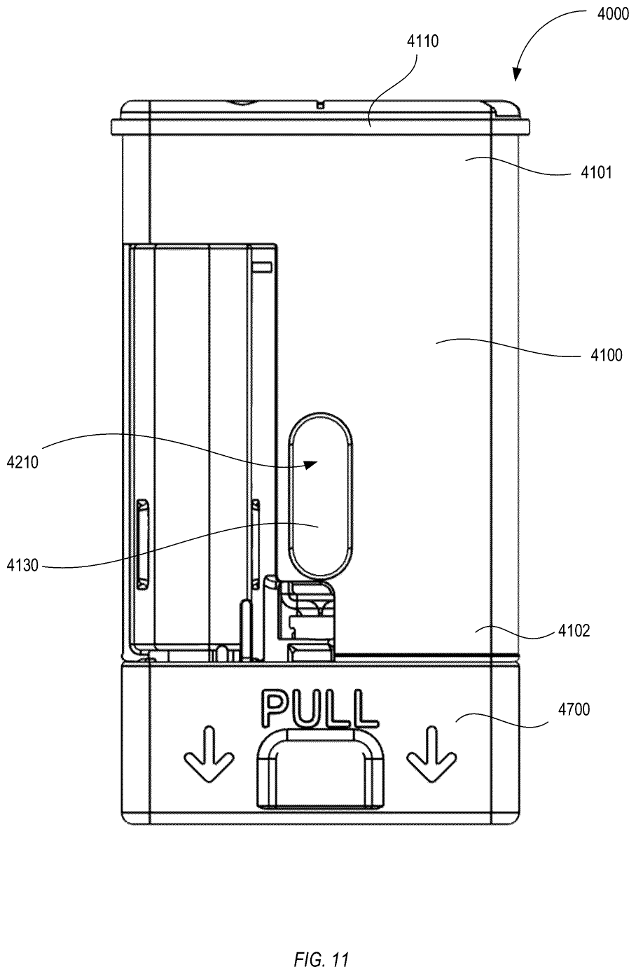

The medical injector 4000 can be moved from the first configuration (FIGS. 11 and 12) to a second configuration (FIG. 28) by moving the safety lock 4700 from a first position to a second position. The safety lock 4700 is moved from a first position to a second position by moving and/or removing the safety lock 4700 distally with respect to the housing 4100. When the safety lock 4700 is moved from the first position to the second position, the safety lock protrusion 4702 is removed from between the extensions 4553 of the release member 4550, thereby enabling the medicament delivery mechanism 4300. As shown in FIG. 11, prior to actuation, a portion of the medicament container assembly 4200 can be viewed via the status aperture 4130. Specifically, the medicament container body 4210 and the contents therein (e.g., the medicament) can be viewed. As described above, in some embodiments, the housing 4100 can include a label or other indicia providing a color strip (against which the medicament can be compared), instructions for viewing or the like. Although not shown in FIG. 11, in some embodiments, a portion of the elastomeric member 4217 is visible via the status aperture 4130.

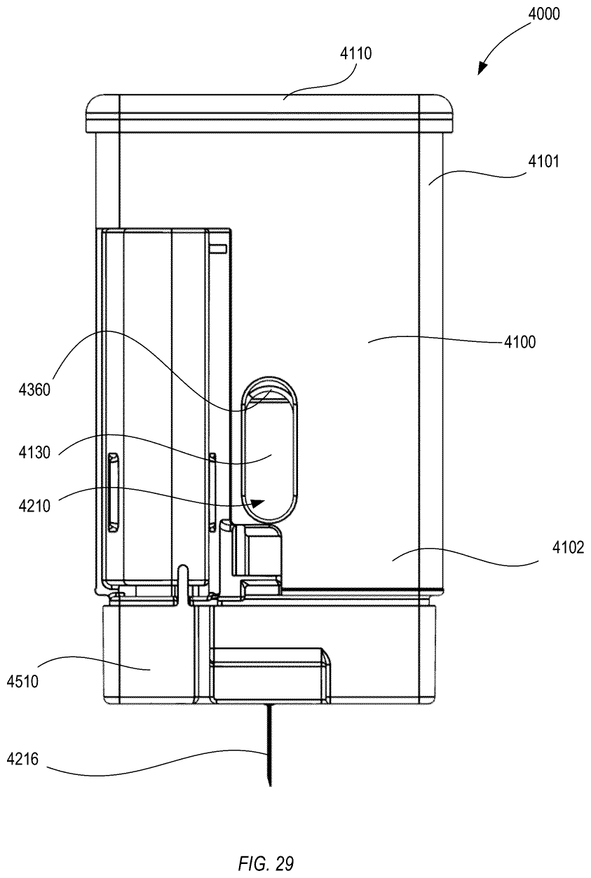

After the safety lock 4700 is moved from the first position to the second position, the medical injector 4000 can be moved from the second configuration (FIG. 28) to a third configuration (FIG. 29) by moving the base 4510 from a first position to a second position. Similarly stated, the medical injector 4000 can be actuated by the system actuator assembly 4500 by moving the base 4510 proximally relative to the housing 4100. The base 4510 is moved from its first position to its second position by placing the medical injector 4000 against the body of the patient and moving the base 4510 with respect to the housing 4100. Specifically, as described above the base includes a “contact portion” (i.e., the distal surface 4523) that can be placed against and/or in contact with the target location. Moving the base 4510 from the first position to the second position causes the base 4510 to engage the extensions 4553 of the release member 4550, thereby moving the extensions 4553 together. The inward movement of the extensions 4553 causes engagement surface of the release member 4550 to become disengaged from the housing 4100, thereby allowing the release member 4550 to be moved proximally along its longitudinal axis as the spring 4576 expands.

When the base 4510 is moved from the first position to the second position, the system actuator assembly 4500 actuates the medicament delivery mechanism 4300, thereby placing the medical injector 4000 in its fourth configuration (i.e., the needle insertion configuration), as shown in FIGS. 29-31. More particularly, when the medical injector 4000 is in its fourth configuration, the puncturer 4575 of the release member 4550 is in contact with and/or disposed through the frangible seal 4413 of the gas container 4410.

After the frangible seal 4413 has been punctured, an actuating portion of a compressed gas flows from the gas container 4410, via the gas passageway 4135 and into the medicament cavity 4139 to begin a first phase of expansion (i.e., movement of the carrier assembly 4390 as the device transitions from the third configuration to the fourth configuration). The gas applies gas pressure to flange 4214 of the medicament container, the delivery control mechanism 4430 and/or the top surface of the carrier body 4360. Because the seals 4371, 4372 of the medicament container assembly 4200, the outer seal 4370 of the carrier assembly 4390, and the seals of the delivery control mechanism 4430 maintain the medicament cavity 4139 fluidically isolated from the exterior of the device, the gas pressure exerts a force to move the carrier assembly 4390 distally within the medicament cavity 4139, as shown in FIGS. 30 and 31. The medicament container body 4210 and delivery control mechanism 4430 also move distally together with the carrier assembly 4390. In this manner, the movement of the needle 4216 in a distal direction causes the distal end portion of the needle 4216 to exit the housing 4100 and enter the body of a patient prior to administering the medicament. In some embodiments, the gas container 4410 can contain a pressurized gas at about 1000 psi prior to the frangible seal 4413 being punctured. Once the frangible seal 4413 has been punctured, the pressurized gas is released into the medicament cavity 4139 is pressurized to about 500 psi at the start of the third configuration (i.e., prior to gas vent assembly 4310 and the carrier assembly 4390 actuating). In some embodiments, the compressed gas supplied by the gas container 4410 is an argon gas. In some embodiments the compressed gas supplied by the gas container 4410 is a refrigerant such as R134a.

As shown in FIGS. 30 and 31, when the device moves from the third configuration to the fourth configuration, the gas vent assembly 4310 expands from its collapsed configuration (FIGS. 22A and 28) to a partially expanded configuration. Notably, in the partially expanded configuration, gas pressure within the medicament cavity 4139 acts on an underside of the proximal end portion 4342 and the valve portion 4345 is maintained in a scaled position within the opening 4112 and the O-ring 4113. Thus, the medicament cavity 4139 is maintained in fluidic isolation.

When the needle 4216 has extended by a desired distance, the distal surface 4365 of the carrier body 4360 contacts the shoulder portion 4106 of the housing 4100 to limit further distal movement of the carrier assembly 4390 within the housing 4100. When the distal movement of the carrier assembly 4390 is prevented, the first phase of expansion is complete. The gas within the medicament cavity 4139 (i.e., the housing gas chamber) continues to travel through the delivery control mechanism 4430 to apply gas pressure to the elastomeric member 4217 to begin a second phase of expansion. During the first phase of expansion, the flow restriction provided by the delivery control mechanism 4430 prevents movement of the elastomeric member 4217 prior to the carrier body 4360 contacting the shoulder portion 4106. In some embodiments, the delivery control mechanism 4430 can permit gas to pass through the flow restriction member 4450 but not build enough pressure to move the elastomeric member 4217 during the first phase of expansion. In some embodiments, the pressurized gas in the medicament cavity 4139 drops to about 90-100 psi at the end of the fourth configuration (i.e., when the housing 4100 and shoulder portions 4106 limits the carrier assembly 4390 from any further distal movement).

As generally shown in FIG. 13A, the gas from the medicament cavity 4139 passes through and is regulated by the flow restriction member 4450. The gas passing through the flow restriction member 4450 travels through the through-hole 4433c and along an interior passage 4438 between the second inner cylindrical surface 4431d and sidewall 4334 of the second member 4330. The interior passage 4438 between the second member 4330 and the first body portion 4431 defines an annulus-shaped passageway. In some embodiments, the second member 4330 and/or the first body portion 4431 can include one or more dimple or bumper portions extending along the axis A₁ to maintain separation between the second member 4330 and/or the first body portion 4431 and to the prevent interior passage 4438 (sec FIG. 15) from being sealed or restricted. In some embodiments, a surface of the distal end portion 4331 facing the first body portion 4431 includes one or more grooves or ridges. In some embodiments, a surface of the distal end portion 4331 facing the first body portion 4431 includes a textured surface.

After passing through the interior passage 4438, the gas enters a medicament body gas chamber 4440 sealed between a distal side of the O-ring 4437 and a proximal side of the elastomeric member 4217. This causes the elastomeric member 4217 (and therefore the first member 4320 of the gas vent assembly 4310) to move in the distal direction within the medicament container body 4210. Distal movement of the elastomeric member 4217 generates a pressure upon the medicament contained within the medicament container assembly 4200, thereby allowing at least a portion of the medicament to flow out of the medicament container 4200 via the needle 4216. The medicament is delivered to a body of a user via the medicament delivery path defined by the medicament container 4200 and the needle 4216. As the elastomeric member 4217 travels to dispense medicament, the gas vent assembly 4310 expands from the partially expanded configuration to a fully expanded configuration and the medical injector is in its fifth configuration (FIGS. 33 and 34). In some embodiments, the medicament body gas chamber 4440 is pressurized to about 10 to 20 psi to begin actuation the elastomeric member 4217.

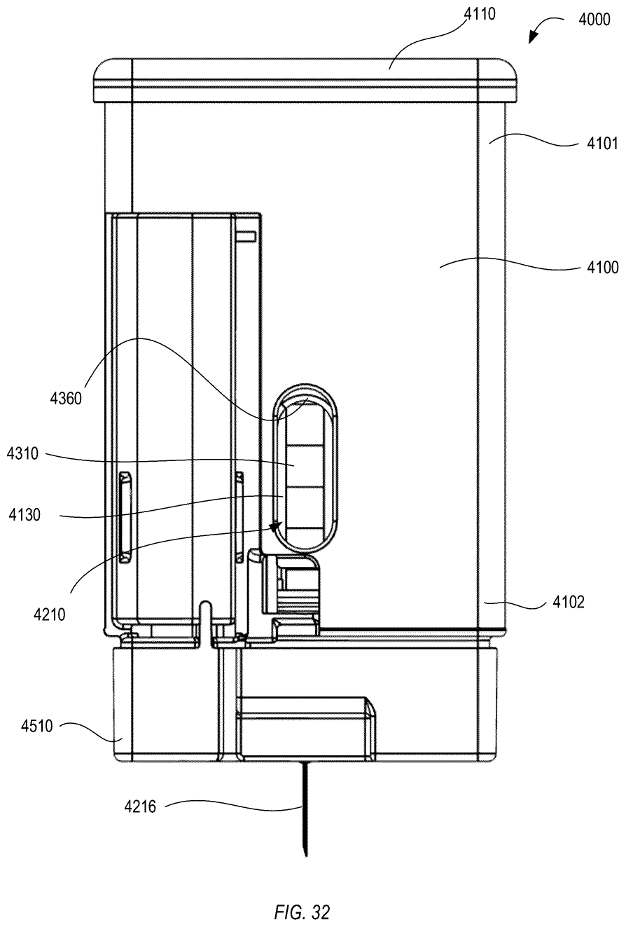

As shown in FIG. 32, when the medical injector 4000 is in its fifth configuration and/or is transitioning to its sixth configuration, a portion of the medicament container assembly 4200, a portion of the carrier body 4360, and a portion of the gas vent assembly 4310 can be viewed via the status aperture 4130. As described above, in some embodiments, the housing 4100 can include a label or other indicia providing a color strip to assist the user in identifying the carrier, providing instructions for viewing, or the like. Although not shown in FIG. 11, in some embodiments, a portion of the elastomeric member 4217 is visible via the status aperture 4130 when the medical injector 4000 is in its fifth configuration or in its sixth configuration to indicate that the delivery of medicament is nearing completion or has completed.

As shown in FIGS. 33 and 34, as the elastomeric member 4217 moves distally, the gas vent assembly 4310 moves together with the elastomeric member 4217 in its fully expanded configuration. Once the gas vent assembly 4310 is in the fully expanded configuration and begins to pull on the valve portion 4345, the medical injection 4000 is in its sixth configuration. In this sixth configuration, the elastomeric member 4217 continues to move a predetermined distance within the medicament container body 4210 (corresponding to a remainder of the desired dose), the valve portion 4345 is moved from within the opening 4112 thereby allowing the pressurized gas contained within the housing gas chamber (i.e., the volume within the medicament cavity 4139 between the proximal end of the housing 4100 and the surface of the carrier 4360) to escape via the passageway 4346 and the opening 4112. More specifically, the pressure applied by the gas in the medicament body gas chamber 4440 on the elastomeric member 4217 is greater than the pressure applied by the gas on the underside of the proximal end portion 4342 and the frictional forces acting on the guide surface 4344. In some embodiments, the medicament body gas chamber 4440 is pressurized to about 30-50 psi to actuate the valve portion 4345.

After the gas pressure within the medicament cavity 4139 decreases below a certain level, the force exerted by the 4 4380 on the carrier body 4360 is sufficient to cause the carrier body 4360 to move proximally within the housing 4100 (i.e., to retract). This places the medical injector in its seventh configuration (FIGS. 39 and 40). As shown in FIGS. 13, 14B, 39 and 40, an inner diameter of the first inner cylindrical surface 4431c is greater than an outer diameter of the guide surface 4344. In this manner, as the carrier assembly 4390, the gas vent assembly 4310, and the flow restriction assembly 4430 move back towards the proximal end of the housing 4100, the first body portion 4431 bypasses the guide surface 4344 to prevent the valve portion 4345 from contact and closing back on the opening 4112.

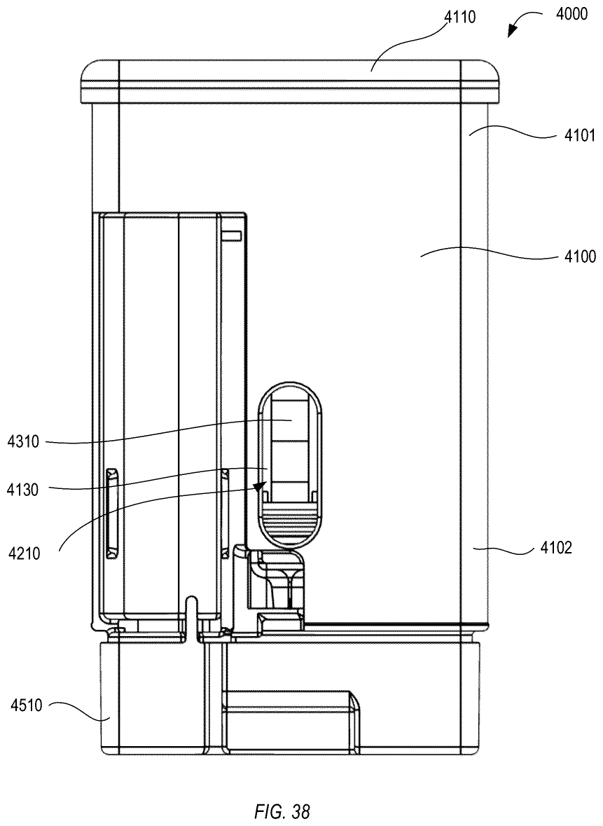

As shown in FIG. 38, when the medical injector 4000 is in its seventh configuration, a portion of the medicament container assembly 4200 can be viewed via the status aperture 4130. Specifically, as shown, the medicament container body 4210 and a portion of the elastomeric member 4217 are visible via the status aperture 4130. As described above, in some embodiments, the housing 4100 can include a label or other indicia providing a color strip to assist the user in identifying the elastomeric member, providing instructions for viewing, or the like. Although not shown in FIG. 38, in some embodiments, a portion of the carrier 4360 is visible via the status aperture 4130 when the medical injector 4000 is in its seventh configuration.

As described above, the medicament delivery mechanism 4300 is considered to be a “pistonless” system. With a pistonless gas-powered auto-injector, the force exerted by the gas can move the medicament container relative to the housing and similarly, can move the elastomeric member 4217 relative to (e.g., within) the medicament container body 4210. In some embodiments, by not including a movable mechanism, a piston, and/or the like, a height of the medical injector 4000 can be reduced relative to, for example, the height of a device that includes a rigid, single length piston.

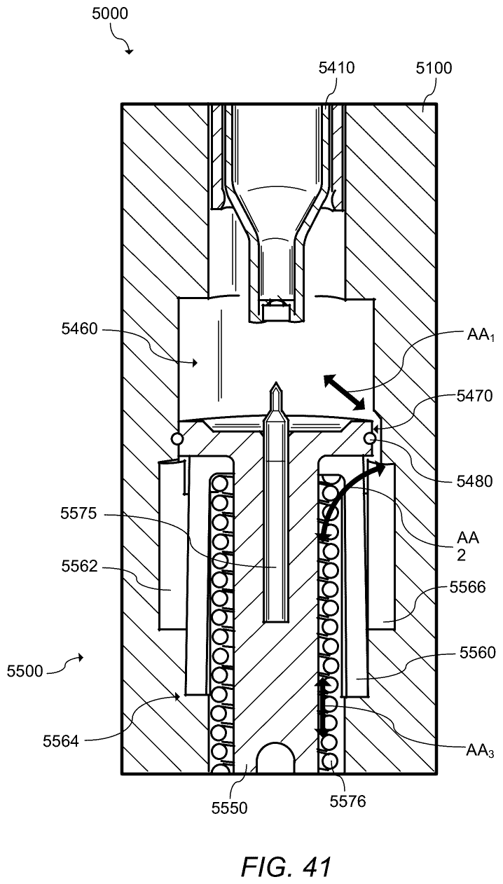

FIGS. 41-43 depict a cross-sectional view of an apparatus 5000 that includes an equalization bypass 5470 and a movable seal member 5480 in three seal positions. In some embodiments, the apparatus 5000 can be a medicament delivery device, such as the medical injector 4000 described herein. Accordingly, the apparatus 5000 can optionally include any of the elements, structures, and/or features described herein with reference to the medical injector 4000.

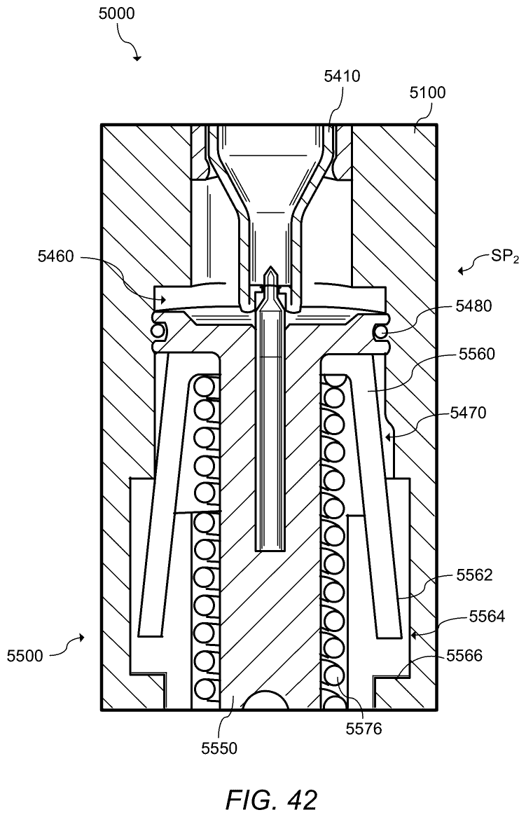

In some embodiments, the apparatus 5000 includes a housing 5100, a gas container 5410, a medicament container assembly (e.g., medicament container assembly 4200), and the movable seal member 5480. The housing 5100 defines a gas chamber 5460 configured to receive a pressurized gas from the gas container 5410 when the apparatus 5000 is actuated. Also, when the apparatus 5000 is actuated, the movable seal member 5480 is configured to move from a first seal position SP₁ (FIG. 41) to a second seal position SP₂ (FIG. 42). In addition to the gas chamber 5460, the housing 5100 also defines an equalization bypass 5470. The equalization bypass 5470 can facilitate fluid communication between the gas chamber 5460 and an exterior volume surrounding the housing 5100 when the movable seal member 5480 is at the first seal position SP₁. For example, as depicted in FIG. 41, fluid communication between the gas chamber 5460 and the exterior volume can occur along the path described by arrows AA₁-AA₃. Accordingly, a volume of gas can pass from the gas chamber 5460, bypass the movable seal member 5480 via the equalization bypass 5470, flow distally between the housing 5100 and a release member 5550 and through a base (e.g., base 4510) of the apparatus 5000 and vice versa. However, when, as depicted in FIG. 42, the movable seal member 5480 is at the second seal position SP₂, the gas chamber 5460 is fluidically isolated from the exterior volume by the movable seal member 5480. In other words, the movable seal member 5480 in the second seal position SP₂ seals the gas chamber 5460, thereby facilitating an increase in gas pressure within the gas chamber 5460.

As depicted in FIGS. 41-43, in some embodiments, the apparatus 5000 includes a system actuator assembly 5500. The system actuator assembly 5500 can include any of the features described herein with reference to the system actuator assembly 4500. In particular, the system actuator assembly 5500 includes a release member 5550, a puncturer 5575 coupled to the release member 5550, and an actuation spring 5576. The release member 5550 is movable within the housing 5100 between a first release member position as depicted in FIG. 41 and a second release member position as depicted in FIG. 42. When the release member 5550 is in the first release member position, the puncturer 5575 is spaced apart from the gas container 5410. The actuation spring 5576 is positioned between a portion of the release member 5550 and the housing 5100 and is configured to move the release member 5550 from the first release member position to the second release member position. The release member 5550 is in a locked configuration and the actuation spring 5576 has a stored-energy configuration prior to actuation of the apparatus 5000. The release member is in a released configuration in response to the actuation force exerted by the actuation spring 5576 when the apparatus 5000 is actuated, and the puncturer 5575 pierces a portion of the gas container 5410 when the release member 5550 is in the second release member position. In other words, the puncturer 5575 is positioned to puncture the gas container 5410 in response to the release member 5550 transitioning from the locked configuration towards the released configuration.

In some embodiments, the movable seal member 5480 (e.g., similar to the sealing member 4574) is coupled to the release member 5550. The movable seal member 5480 can, in some embodiments, be an O-ring that surrounds the release member 5550. However, in some embodiments, the movable seal member 5480 can be a sealing structure (e.g., protrusion, lip, or other similar structure) monolithically formed as part of the release member 5550. The movable seal member 5480 is at the first seal position SP₁ when the release member 5550 is in the first release member position as depicted in FIG. 41. The movable seal member 5480 is in the second seal position SP₂ when the release member 5550 is in the second release member position as depicted in FIG. 42. Said another way, the movable seal member 5480 is coupled to the release member 5550 and is positioned between the release member 5550 and an inner wall of the housing 5100. The movable seal member 5480 is at the first seal position SP₁ when the release member is in the locked condition and is configured to move to the second seal position SP₂ in response to the release member transitioning from the locked configuration towards the released configuration. Accordingly, the movable seal member 5480 fluidically isolates the gas chamber 5460 from the exterior volume following actuation of the apparatus 5000 but prior to the puncturing of the gas container 5410. As such, when discharged from the gas container, the pressurized gas is contained within the sealed gas chamber 5460 and the ability of the pressurized gas to deliver the medicament is unaffected by a loss of gas.

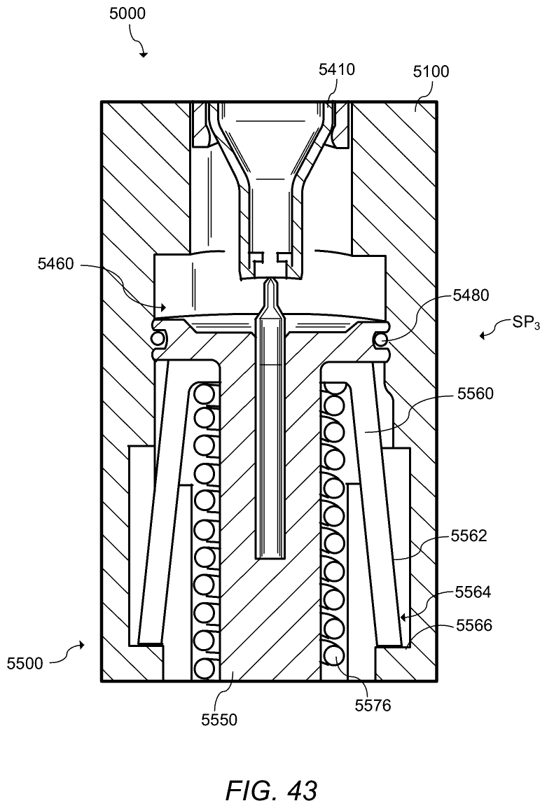

As depicted in FIGS. 41-43, in some embodiments, the system actuator assembly 5500 includes a positioning member 5560. The positioning member 5560 can be coupled to the release member 5550. As depicted in FIG. 41, the positioning member 5560 can have a first shape when the release member 5550 is in the locked configuration. The positioning member 5560 can have a second shape when the release member 5550 is in the released configuration as depicted in FIG. 42. The first shape can, for example, be a compressed configuration in which the positioning member 5560 stores potential energy (e.g., elastic energy), while the second shape can be an expanded configuration resulting from the release of potential energy. However, in some embodiments, the first shape can be an expanded configuration that is compressed to transition to the second shape. In some embodiments, the positioning member 5560 can, for example, include at least one leg portion 5562. The leg portion(s) 5562 can have a distal end portion 5564. The distal end portion 5564 can have a first radial position (FIG. 41) in the first shape and a second radial position (FIGS. 42 and 43) in the second shape. The second radial position can be radially outward of the first radial position. Said another way, the positioning member 5560 can have a first circumference in the first shape that is less than a second circumference in the second shape.

As depicted in FIG. 43, when in the second shape, the positioning member 5560 engages a receiving portion 5566 of the housing 5100. The engagement between the positioning member 5560 and the receiving portion 5562 precludes a movement of the release member 5550. For example, the engagement can preclude a return of the release member 5550 to the first release member position from the second release member position. Said another way, the engagement between the positioning member 5560 and the receiving portion 5566 can limit a motion (e.g., a motion in the distal direction) along an axis of motion (e.g., along a longitudinal axis of the apparatus 5000). In some embodiments, the movable seal member 5480 is, as depicted in FIG. 43, maintained at a third seal position SP₃ when the positioning member 5560 engages the receiving portion 5566 of the housing 5100. In some embodiments, the third seal position SP₃ is between the first seal position SP₁ and the second seal position SP₂ along the axis of motion. The gas chamber 5460 is fluidically isolated from the exterior volume by the movable seal member 5480 when the movable seal member 5480 is at the third seal position SP₃. It should be appreciated that absent the positioning member 5560, the force exerted by the release of pressurized gas from the gas container 5410 could overcome the force exerted by the actuation spring 5576, thereby causing the release member 5550, and the movable seal member 5480, to return to the first release member position, and the first seal position SP₁. Upon such an occurrence, the fluid coupling between the gas chamber 5460 and the exterior volume could be reestablished and the operation of the apparatus 5000 affected.

In some embodiments, the movable seal member 5480 is a first movable seal member. In such an embodiment, the apparatus 5000 can include a second movable seal member that is coupled to a carrier (e.g., outer O-ring 4370 coupled to carrier 4360). Additionally, the apparatus 5000 can include a third movable seal member (e.g., the valve member 4345 configured as a movable seal).

FIG. 44 depicts a cross-sectional view of an apparatus 6000 that includes an equalization bypass 6470 and a movable seal member 6480 in a first seal position SP₁. In some embodiments, the apparatus 6000 can be a medicament delivery device, such as the medical injector 4000, or apparatus 5000 described herein. Accordingly, the apparatus 6000 can optionally include any of the elements, structures, and/or features described herein with reference to the medical injector 4000 or the apparatus 5000.

In some embodiments, the apparatus 6000 includes a housing 6100, a gas container 6410, a medicament container assembly 6200 (e.g., medicament container assembly 4200), and the movable seal member 6480. The medicament container assembly 6200 includes a container body 6210 and an elastomeric member 6217 disposed within the container body 6210. The elastomeric member 6217 is configured to move within the container body 6210 to convey a medicament contained therein. The housing 6100 defines a gas chamber 6460 configured to receive a pressurized gas from the gas container 6410 when the apparatus 6000 is actuated. Also, when the apparatus 6000 is actuated, the movable seal member 6480 is configured to move from a first seal position SP₁ to a second seal position (e.g., a distal seal position). In addition to the gas chamber 6460, the housing 6100 also defines an equalization bypass 6470. The equalization bypass 6470 can facilitate fluid communication between the gas chamber 6460 and an exterior volume surrounding the housing 6100 when the movable seal member 6480 is at the first seal position SP₁. For example, as depicted, fluid communication between the gas chamber 6460 and the exterior volume can occur along the path described by arrow BB. Accordingly, a volume of gas can pass from the gas chamber 6460, bypass the movable seal member 6480 via the equalization bypass 6470, flow distally between the housing 6100 and a carrier 6360 or the medicament container assembly 6200 and through a base (e.g., base 4510) of the apparatus 6000 and vice versa. However, when the movable seal member 6480 is at the second seal position the gas chamber 6460 is fluidically isolated from the exterior volume by the movable seal member 6480. In other words, the movable seal member 6480 in the second seal position seals the gas chamber 6460, thereby facilitating an increase in gas pressure within the gas chamber 6460.

As depicted, in some embodiments, the apparatus 6000 includes a carrier 6360. The carrier 6360 can include any of the features described herein with reference to the carrier 4360. Accordingly, as described herein with reference to carrier 4360, the carrier 6360 can be coupled to the medicament container assembly 6200 and configured to move (e.g., move distally) within the housing 6100. The movement of the carrier 6360 can be in response to the force exerted by the pressurized gas within the gas chambers 6460 following actuation of the apparatus 6000. Accordingly, a proximal surface of the carrier 6360 can define a portion of a boundary of the gas chamber 6460.

In some embodiments, the movable seal member 6480 (e.g., outer O-ring 4370) is coupled to the carrier 6360 and positioned between the carrier 6360 and an inner wall of the housing 6100. The movement of the carrier 6360 in response to the force exerted by the pressurized gas moves the movable seal member 6480 relative to the housing 6100 from the first seal position SP₁ to the second seal position, thereby isolating the gas chambers 6460 from the exterior volume.

In some embodiments, the carrier 6360 can be omitted and the movable seal member 6480 can be coupled directly to the container body 6210 and positioned between the container body 6210 and the inner wall of the housing 6100. The container body 6210 can be movable within the housing 6100 from a first container position to a second container position in response to the force exerted by the pressurized gas within the gas chamber 6460. The movement of the container body 6210 in response to the force exerted by the pressurized gas moves the movable seal member 6480 relative to the housing 6100 from the first seal position SP₁ to the second seal position to isolate the gas chambers 6460 from the exterior volume.

In some embodiments, the movable seal member 6480 is a first movable seal member. In such an embodiment, the apparatus 6000 can include a second movable seal member that is coupled to a release member of a system actuator assembly (e.g., scaling member 4574 coupled to release member 4550). Additionally, the apparatus 6000 can include a third movable seal member (e.g., the valve member 4345 configured as a movable seal).

FIG. 45 depicts a cross-sectional view of an apparatus 7000 that includes an equalization bypass 7470 and a movable seal member 7480 in a first seal position SP₁. In some embodiments, the apparatus 7000 can be a medicament delivery device, such as the medical injector 4000, apparatus 5000, or apparatus 6000 described herein. Accordingly, the apparatus 7000 can optionally include any of the elements, structures, and/or features described herein with reference to the medical injector 4000, the apparatus 5000, or the apparatus 6000.

In some embodiments, the apparatus 7000 includes a housing 7100, a gas container 7410, a medicament container assembly 7200 (e.g., medicament container assembly 4200), and the movable seal member 7480. The medicament container assembly 7200 includes a container body 7210 and an elastomeric member 7217 disposed within the container body 7210. The elastomeric member 7217 is configured to move within the container body 7210 to convey a medicament contained therein. The housing 7100 defines a gas chamber 7460 configured to receive a pressurized gas from the gas container 7410 when the apparatus 7000 is actuated. Also, when the apparatus 7000 is actuated, the movable seal member 7480 is configured to move from a first seal position SP₁ to a second seal position (e.g., a distal seal position). In addition to the gas chamber 7460, the housing 7100 also defines an equalization bypass 7470. The equalization bypass 7470 can facilitate fluid communication between the gas chamber 7460 and an exterior volume surrounding the housing 7100 when the movable seal member 7480 is at the first seal position SP₁. For example, as depicted, fluid communication between the gas chamber 7460 and the exterior volume can occur along the path described by arrow CC. Accordingly, a volume of gas can pass from the gas chamber 7460, bypass the movable seal member 7480 via the equalization bypass 7470, flow proximally out of the housing 7100 via a vent opening 7112 and vice versa. However, when the movable seal member 7480 is at the second seal position the gas chamber 7460 is fluidically isolated from the exterior volume by the movable seal member 7480. In other words, the movable seal member 7480 in the second seal position seals the gas chamber 7460, thereby facilitating an increase in gas pressure within the gas chamber 7460. In some embodiments, the movable seal member 7480 is configured to move from the first position SP₁ to the second position in response to a force exerted by the pressurized gas within the gas chamber 7460.

In some embodiments, the apparatus 7000 includes a gas vent assembly 7310. The gas vent assembly can include any of the features described herein with reference to the gas vent assembly 4310. The gas vent assembly 7310 is configured to selectively place the gas chamber 7460 in fluid communication with the exterior volume. The movable seal member 7480 is configured as a valve member (e.g., valve member 4345). The movable seal member 7480 is configured to occlude and/or seal both the equalization bypass 7470 and the vent opening 7112 defined by the housing 7100. Prior to actuation, the movable seal member 7480 (i.e., the valve member) is at the first seal position SP₁. In response to a force exerted by the pressurized gas within the gas chamber 7460, the movable seal member 7480 is configured to move (e.g., move proximally) to the second seal position, thereby sealing the vent opening 7112 and fluidically isolating the gas chamber 7460 from the exterior volume. The movable seal member 7480 (i.e., the valve member) is operably coupled to the elastomeric member of the medicament container assembly 7200 via an expandable assembly (e.g., the first member 4320, the second member 4330, and the third member 4340). The expandable assembly is configured to transition from a first configuration to a second configuration when the elastomeric member moves within the container body. In response to the movement of the elastomeric member, the movable seal member 7480 (i.e., the valve member) moves from the second seal position to a third seal position when the expandable assembly transitions from the first configuration to the second configuration. The movement of the movable seal member 7480 to the third seal position releases the pressurized gas from the gas chamber 7460 to the exterior volume as described herein.

Said another way, prior to actuation, the movable seal member 7480 is at the first seal position SP₁ and the gas chamber 7460 is in fluid communication with the exterior volume via the equalization bypass 7470 and the vent opening 7112. Upon actuation of the apparatus 7000, pressurized gas is released from the gas container 7410. An initial portion of the pressurized gas enters the gas chamber 7460 and exerts a force on the movable seal member 7480 causing the movable seal member 7480 to move to the second seal position. In the second seal position, the movable seal member 7480 fluidically isolates the gas chamber 7460 from the exterior volume. The pressurized gas also exerts a force on the elastomeric member causing the elastomeric member to move to dispense a portion of the medicament contained within the medicament container assembly 7200. Following a sufficient degree of movement of the elastomeric member, the movable seal member 7480 is moved from the second seal position to a third seal position, which can be the same as the first seal position SP₁, to release the pressurized gas from the gas chamber 7460 to the exterior volume.

In some embodiments, the movable seal member 7480 is a first movable seal member. In such an embodiment, the apparatus 7000 can include a second movable seal member that is coupled to a release member of a system actuator assembly (e.g., scaling member 4574 coupled to release member 4550). Additionally, the apparatus 7000 can include a third movable seal member coupled to a carrier (e.g., outer O-ring 4370 coupled to carrier 4360).

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

For example, in some embodiments, a medicament delivery device can include two or more medicament containers, each having a delivery member through which the medicament therein can be delivered. Such embodiments can accommodate the delivery of viscous medicaments and/or large volumes of medicament (e.g., >1 mL dose) by delivering portions of the overall dose in parallel. Specifically, as discussed above with respect to Eq. 1, the needle length (L) and the needle gauge (identified as the radius R of the needle lumen) can have a profound impact on the pressure needed to deliver a desired volume of medicament therethrough. Thus, by using a “parallel delivery” device of the types shown and described herein, delivery of viscous medicaments, such as certain large or macromolecular injectables that include carbohydrate-derived formulations, lipids, nucleic acids, hyaluronidase, proteins/peptides (e.g. monoclonal antibodies) and other biotechnologically-derived medicaments, can be facilitated. Any of the gas venting mechanisms, electronic circuit systems, or other components described herein can be included in a dual container device of the types shown and described in the '4345 PCT or the '0040 PCT.

For example, any of the elastomeric members described herein can be constructed from any suitable material or combination of different materials. For example, in some embodiments, at least a portion of any of the elastomeric members described herein can be coated. Such coatings can include, for example, polydimethylsiloxane. In some embodiments, at least a portion of any of the elastomeric members described herein can be coated with polydimethylsiloxane in an amount of between approximately 0.02 mg/cm² and approximately 0.80 mg/cm².

Any of the medicament container assemblies described herein can have any suitable size (e.g., length and/or diameter) and can contain any suitable volume of the medicament. In some embodiments, any of the medicament container assemblies described herein can be a prefilled (or prefillable) syringe, such as those manufactured by Becton Dickinson, Gerresheimer, Ompi Pharma or others. For example, in some embodiments, the medicament container assembly 4200 (and any of the medicament container assemblies described herein) can be a Becton Dickinson “BD Hypak Physiolis” prefillable syringe containing any of the medicaments described herein. Moreover, any of the medicament delivery devices and/or medical injectors described herein can be configured to inject any suitable dosage such as, for example, a dose of up to 1 mL of any of the medicaments described herein. In other embodiments, any of the medicament delivery devices and/or medical injectors described herein can be configured to inject a dose of up to 2 mL. 3 mL, 4 mL, 5 mL, or more of any of the medicaments described herein.

Any of the container bodies described herein can be constructed from glass, and can be fitted and/or coupled to any suitable needle. For example, in some embodiments, any of the container bodies described herein (including the container body 4210) can be coupled to a needle having any suitable size. Any of the medicament container assemblies and/or prefilled syringes described herein can be coupled to a needle having a gauge size of 21 gauge, 22 gauge, 23 gauge, 24 gauge, 25 gauge, 26 gauge, 27 gauge, 28 gauge, 29 gauge, 30 gauge, or 31 gauge. Any of the medicament container assemblies and/or prefilled syringes described herein can be coupled to a needle having any suitable length, such as, for example, a length of about 0.2 inches, about 0.27 inches, about 0.38 inches, about 0.5 inches, about 0.63 inches, about 0.75 inches, or more. In some embodiments, any of the medicament containers and/or prefilled syringes described herein can be coupled to a 29 gauge needle having a length of approximately 0.5 inches. Moreover, any of the medicament containers and/or prefilled syringes described herein can include a staked needle at the distal end thereof.

For example, any of the medical injectors shown and described herein can include a base (or distal actuator) having a mechanism for cooling the surface of the target injection site. By cooling the target injection site, patient comfort during an injection operation can be improved. Such cooling mechanisms can include, for example, an electronic cooler (e.g., a thermo-electric cooler) that is triggered upon removal of a safety guard, a chemical or spray that is emitted by the base upon removal of the safety guard, or any other suitable mechanism.

Any of the medical injectors shown and described herein can include a base (or distal actuator) having a mechanism for expanding, stretching or otherwise pulling taut a patient's skin at or near an injection site. In other embodiments, the base (or distal actuator) of any of the injectors described herein can include a mechanism that increases the surface area of the base (or distal actuator) against the injection site. For example, in some embodiments a base can include a series of grips, protrusions, microneedles, or the like that can grip the skin and expand to stretch the surface prior to actuation and/or injection or allow for a large surface area of contact against the skin for added stability for injectate administration. In other embodiments, a base can include a series of grips, protrusions, microneedles, or the like that can grip the skin and pinch the surface together prior to actuation and/or injection. Such a base can include a dome or other structure to pinch certain portions of the anatomy, such as, for example, the abdomen.

Although the medicament injectors shown and described above include a delivery mechanism (e.g., 4300) including the release of a pressurized gas, in other embodiments, a medicament delivery device can include any suitable method of delivery of a medicament disposed within. For example, in some embodiments, any of the devices described herein can include a mechanical energy storage (e.g. spring, gears, racks, pinions, pulleys, or the like) member, rather than a compressed gas container. In other embodiments, any of the devices described herein can include any other suitable energy storage member (e.g., magnetic, electrical, propellant based, chemical reaction based, or the like).

While the medical injectors herein are described as being “pistonless” gas-powered auto-injectors, in other embodiments, any of the medical injectors can include any suitable energy storage member configured to produce a force directly on a medicament container and/or a carrier (as described, for example, in the '849 patent). For example, in some embodiments, a medical injector can include one or more bias members, springs, and/or any other suitable mechanical drives (as described above) configured to exert a force on one or more medicament containers. By way of example, a medical injector can include a first spring configured to produce a force on a first medicament container and a second spring configured to produce a force, substantially equal to the force produced by the first spring, on a second medicament container. Moreover, the first spring and the second spring can be actuated substantially concurrently and/or via the same actuation event such that the first spring and second spring move the first medicament container and the second medicament container substantially concurrently.

Although particular injection events, mechanisms, devices, and/or components have been described herein, it is to be understood that they have been presented by way of example and not limitation. That is to say, an auto-injector can include more than one medicament container and can be configured to deliver at least one dose of a medicament to a patient in response any suitable actuation event and/or the like.

Any of the devices and/or medicament containers shown and described herein can be constructed from any suitable material. Such materials include glass, plastic (including thermoplastics such as cyclic olefin copolymers), or any other material used in the manufacture of prefilled syringes containing medications.

Any of the devices and/or medicament containers shown and described herein can contain and/or deliver a wide array of large or macromolecular injectables that include carbohydrate-derived formulations, lipids, nucleic acids, nucleic acids, hyaluronidase, proteins/peptides (e.g. monoclonal antibodies) and other biotechnologically-derived medicaments. For example, anti-tumor necrosis factor agents such as infliximab, etanercept, adalimumab, golimumab, natalizumab, vedolizumab, and certolizumab can be administered using the described auto-injector heroin, Other macromolecular injectable medications that can be administered using the device and/or medicament containers shown and described herein include viscous medicaments that target pro-inflammatory cytokines (e.g. IL-1, IL-2, IL-4, IL-5, IL-6, IL-12, IL-13, IL-23, IL-17, IL-21, IL-23A, and associated receptors) including dupilumab, sarilumab, mepolizumab, benralizumab, reslizumab, lebrikizumab, ustekinumab, anrunkinzumab, bertilimumab, tralokinumab, and risankizumab. Large anti-adhesion molecules to treat a variety of diseases may be administered using the device and/or medicament containers shown and described herein including etrolizumab and vatelizumab. Still other large and viscous monoclonal antibodies that may be administered using the device and/or medicament containers shown and described herein include tezepelumab, anifrolumab, omalizumab, and proprotein convertase subtilisin kexin type 9 (PCSK9) inhibitors including alirocumab and evolocumab.

Any of the devices and/or medicament containers shown and described herein can include any suitable medicament or therapeutic agent. In some embodiments, the medicament contained within any of the medicament containers shown herein can be a vaccine, such as, for example, an influenza vaccine, a hepatitis vaccine, a haemophilus influenza Type B (HiB) vaccine, a measles vaccine, a mumps vaccine, a rubella vaccine, or combination vaccine (e.g. measles, mumps and rubella, quadrivalent, or hexavalent vaccines), a polio vaccine, a human papilloma virus (HPV) vaccine, a tetanus vaccine, a diphtheria vaccine, a pertussis vaccine, a bubonic plague vaccine, a yellow fever vaccine, a cholera vaccine, a malaria vaccine, a smallpox vaccine, a pneumococcal vaccine, a rotavirus vaccine, a varicella vaccine, a dengue fever vaccine, a rabies vaccine and/or a meningococcus vaccine. In other embodiments, the medicament contained within any of the medicament containers shown herein can be a catecholamine, such as epinephrine. In other embodiments, the medicament contained within any of the medicament containers shown herein can be an opioid receptor antagonist, such as naloxone, including any of the naloxone formulations described in U.S. Pat. No. 8,627,816, entitled “Medicament Delivery Device for Administration of Opioid Antagonists Including Formulation for Naloxone,” filed on Feb. 28, 2011. In yet other embodiments, the medicament contained within any of the medicament containers shown herein can include peptide hormones such as insulin and glucagon; human growth hormone (HGH); sumatriptan; a corticosteroid such as dexamethasone; ondansetron; an opioid agonist receptor modulators such as fentanyl; a partial agonist opioid receptor modulators such as buprenorphine; a mixed agonist/antagonist opioid receptor modulator such as nalbuphine; a benzodiazepine such as diazepam, midazolam or lorazepam; erythropoiesis-stimulating agents (ESA) such as darbepoetin alfa; immunoglobulins including dual-variable domain immunoglobulins; interferons; anti-tumor; recombinant human granulocyte colony-stimulating factor (GCSF) such as pegfilgrastim; icatibant; and other therapies suitable for injection in mammals. In yet other embodiments, the medicament contained within any of the medicament containers shown herein can be a placebo substance (i.e., a substance with no active ingredients), such as water.

The medicament containers and/or medicament delivery devices disclosed herein can contain any suitable amount of any medicament. For example, in some embodiments, a medicament delivery device as shown herein can be a single-dose device containing an amount medicament to be delivered of approximately 0.4 mg. 0.8 mg. 1 mg, 1.6 mg or 2 mg. As described above, the fill volume can be such that the ratio of the delivery volume to the fill volume is any suitable value (e.g., 0.4, 0.6 or the like). In some embodiments, an electronic circuit system can include “configuration switch” that, when actuated during the assembly of the delivery device, can select an electronic output corresponding to the dose contained within the medicament container.

In some embodiments, a medical injector can include two prefilled syringes, each containing up to 1 mL of medicament (or more), and each having a needle. Such devices (e.g., “dual container devices”) are shown and described in the '4345 PCT, which is incorporated by reference herein. Upon actuation of the device (as described above), a single energy storage member (e.g., a compressed gas container) can release energy to move the two containers within the housing in substantially the same operation to inject the two needles. The force produced by the energy storage member can further inject the medicament from each container. In such embodiments, the two containers can include either the same medicament or two different medicaments. For example, a dual container device can be filled with and/or used to inject methotrexate (from one container) and tocilizumab (in the other container) for the treatment of rheumatoid arthritis. In some embodiments, a dual container device can be filled with and/or used to inject tocilizumab and methotrexate for the treatment of rheumatoid arthritis, adalimumab and methotrexate for the treatment of psoriasis or rheumatoid arthritis, etanercept and methotrexate for the treatment of psoriatic arthritis, belimumab and rituximab for the treatment of Primary Sjogren's Syndrome, lanrcotide autogel and pegvisomant for the treatment of acromegaly, narlaprevir and ritonavir for the treatment of chronic hepatitis C, alemtuzumab and rituximab for the treatment of chronic lymphocytic leukemia, pertuzumab and trastuzumab for the treatment of HER2-Positive early breast cancer, long-acting insulin glargine and fast-acting insulin lispro for the treatment of Type 2 diabetes, pramlintide and insulin for the treatment of Type 1 diabetes, insulin glargine and insulin lispro for the treatment of Type 1 diabetes, mosunetuzumab and atezolizumab for the treatment of neoplasm, nivolumab and tumor-infiltrating lymphocytes with interleukin-2 for the treatment of metastatic melanoma, pertuzumab and trastuzumab for the treatment of HER2 positive early breast cancer, ocrelizumab and recombinant human hyaluronidase for the treatment of multiple sclerosis, daratumumab recombinant human hyaluronidase for the treatment of multiple myeloma, nivolumab and recombinant human hyaluronidase for the treatment of metastatic tumors, and insulin lispro and recombinant human hyaluronidase for the treatment of diabetes mellitus.

Any of the medicament containers described herein can include any suitable elastomeric member and/or plunger. For example, an elastomeric member can be formulated to be compatible with the medicament contained within a medicament container. Moreover, a medicament container can include any number of elastomeric members. For example, in some embodiments, a medicament container can include a dry portion of a medicament and a fluid portion of the medicament, configured to be mixed before injection. The piston portion of the medicament delivery mechanism can be configured to engage multiple elastomeric members associated with the portions of the medicament. In this manner, multiple elastomeric members can be engaged to mix the dry portion with the fluid portion of the medicament before the completion of an injection event. In some embodiments, for example, any of the devices shown and described herein can include a mixing actuator similar to the mixing actuators shown and described in U.S. Pat. No. 9,173,999, entitled “Devices and Methods for Delivering Medicaments from a Multi-Chamber Container,” filed Jan. 25, 2012, which is incorporated herein by reference in its entirety.

Although the injectors described herein have been shown and described as including mechanisms for needle retraction, in other embodiments any of the injectors shown and described herein can include a needle shield that extends distally after the injection to cover the exposed needle. Such a design may be used, for example, in a “pistonless” design as discussed above. For example, in some embodiments, a base of a medical injector (e.g. the base 4510) can be (or include) an extending portion that, upon completion of the injection, extends distally to cover the needle. In some such embodiments, the gas vent assembly can divert all or a portion of the pressurized gas to a volume within the housing such that the diverted gas exerts a force on the base (or a portion of the base) to cause the base (or portion of the base) to extend distally to cover the needle. In other such embodiments, a spring, biasing member, or retraction member can propel the base (or portion of the base) distally.

Although the gas vent assembly 4310 is shown and described herein as moving a valve portion relative to a seal to selectively place an internal gas chamber in fluid communication with an external volume, in other embodiments, any of the gas vent assemblies disclosed herein can be operable to vent all or a portion of the pressurized gas to a second region within the housing. Further, any of the gas vent assemblies disclosed herein can include any suitable valve arrangement. For example, in some embodiments a gas vent assembly and/or a portion a housing can include a tear-through seal that is punctured or torn when a portion of a medicament carrier or a portion of an elastomeric member moves past a specific point during a delivery event. In other embodiments, a gas vent assembly and/or a portion a housing can include a movable valve member (e.g., a poppet, ball, or the like) that is moved to release pressure when a portion of a medicament carrier or a portion of an elastomeric member moves past a specific point during a delivery event.

Although various embodiments have been described as having particular features and/or combinations of components, other embodiments are possible having a combination of any features and/or components from any of embodiments where appropriate. For example, any of the devices shown and described herein can include an electronic circuit system as described herein.

What is claimed is:

1. An apparatus, comprising:
a housing defining a gas chamber and an equalization bypass, the housing being surrounded by an exterior volume;
a gas container disposed within the housing, the gas container configured to produce a pressurized gas within the gas chamber when the apparatus is actuated;
a medicament container assembly disposed within the housing, the medicament container assembly including a container body and an elastomeric member disposed within the container body, the elastomeric member configured to move within the container body to convey a medicament contained therein in response to a force exerted by the pressurized gas within the gas chamber; and
a movable seal member disposed within the housing, the movable seal member configured to move from a first seal position to a second seal position, wherein:
the gas chamber is in fluid communication with the exterior volume via the equalization bypass when the movable seal member is at the first seal position, and
the gas chamber is fluidically isolated from the exterior volume by the movable seal member when the movable seal member is at the second seal position.

2. The apparatus of claim 1, further comprising:
a system actuator assembly including a release member and a puncturer coupled to the release member, wherein:
the release member is movable within the housing between a first release member position and a second release member position,
the puncturer is spaced apart from the gas container when the release member is in the first release member position, the puncturer piercing a portion of the gas container when the release member is at the second release member position, and
the movable seal member is coupled to the release member, the movable seal member being at the first seal position when the release member is at the first release member position, the movable seal member being at the second seal position when the release member is at the second release member position.

3. The apparatus of claim 1, further comprising:
a system actuator assembly including a release member, a puncturer, and an actuation spring disposed within the housing, wherein:
the release member is movable within the housing,
the release member is in a locked configuration and the actuation spring has a stored-energy configuration prior to actuation of the apparatus, the release member being in a released configuration in response to an actuation force being exerted by the actuation spring on the release member when the apparatus is actuated,
the puncturer is coupled to the release member and positioned to puncture the gas container in response to the release member transitioning from the locked configuration towards the released configuration,
the movable seal member is coupled to the release member and is positioned between the release member and an inner wall of the housing,
the movable seal member is at the first seal position when the release member is in the locked configuration, and
the movable seal member is configured to move to the second seal position in response to the release member transitioning from the locked configuration towards the released configuration.

4. The apparatus of claim 3, wherein:
the system actuator assembly includes a positioning member coupled to the release member;
the positioning member has a first shape when the release member is in the locked configuration and a second shape when the release member is in the released configuration, the positioning member engages a receiving portion of the housing when in the second shape;
the movable seal member is maintained at a third seal position when the positioning member engages the receiving portion of the housing; and
the gas chamber is fluidically isolated from the exterior volume by the movable seal member when the movable seal member is at the third seal position.

5. The apparatus of claim 4, wherein the third seal position is between the first seal position and the second seal position along a path of motion.

6. The apparatus of claim 1, further comprising:
a carrier coupled to the medicament container assembly and configured to move within the housing in response to the force exerted by the pressurized gas, a proximal surface of the carrier defining a portion of a boundary of the gas chamber.

7. The apparatus of claim 6, wherein:
the movable seal member is coupled to the carrier and is positioned between the carrier and an inner wall of the housing; and
the movement of the carrier in response to the force exerted by the pressurized gas moves the movable seal member relative to the housing from the first seal position to the second seal position to isolate the gas chamber from the exterior volume.

8. The apparatus of claim 1, further comprising:
a gas vent assembly configured to selectively place the gas chamber in fluid communication with the exterior volume, the gas vent assembly including a valve member configured to seal a vent opening defined by the housing, wherein:
the valve member occludes the vent opening when at a first valve position prior to actuation of the apparatus,
the valve member is configured to move within the housing from the first valve position to a second valve position in response to the movement of the elastomeric member, and
the gas chamber is in fluid communication with the exterior volume via the vent opening when the valve member is at the second valve position.

9. The apparatus of claim 8, further comprising:
an expandable assembly having a first member, a second member and a third member, the first member coupled to the elastomeric member, the second member coupled between the first member and the third member, the third member coupled to the valve member, the expandable assembly configured to transition from a first configuration to a second configuration when the elastomeric member moves within the container body; and
the valve member moves from the first valve position to the second valve position when the expandable assembly transitions from the first configuration to the second configuration to release the pressurized gas from the gas chamber to the exterior volume.

10. The apparatus of claim 8, wherein the vent opening is sized to maintain the force of the pressurized gas within the gas chamber at a magnitude that is greater than a force applied by a retraction spring during a time to dispense the medicament, the force of the pressurized gas decreasing to a magnitude that is less than a force applied by the retraction spring concurrent with a completion of the dispensing of the medicament.

11. The apparatus of claim 10, wherein the vent opening is sized such that the pressurized gas causes a first 20% of the medicament to dispense over a first time duration and a last 20% of the medicament to dispense over a second time duration that is less than two times the first time duration.

12. The apparatus of claim 8, wherein the movable seal member is a first movable seal member, the apparatus further comprising:
a carrier coupled to the medicament container assembly and configured to move within the housing in response to the force exerted by the pressurized gas, a proximal surface of the carrier defining a first portion of a boundary of the gas chamber; and
a second movable seal member coupled to the carrier and positioned between the carrier and an inner wall of the housing, the second movable seal member defining a second portion of the boundary of the gas chamber.

13. The apparatus of claim 1, wherein:
the movable seal member is configured to move from the first seal position to the second seal position in response to a force exerted by the pressurized gas within the gas chamber; and
the movable seal member is configured to move from the second seal position to a third seal position in response to the movement of the elastomeric member, the gas chamber being in fluid communication with the exterior volume via the equalization bypass when the movable seal member is at the third seal position.

14. The apparatus of claim 13, further comprising:
a gas vent assembly configured to selectively place the gas chamber in fluid communication with the exterior volume, the gas vent assembly including a valve member configured as the movable seal member and a vent opening defined by the housing, the valve member configured to seal the vent opening, wherein the gas chamber is in fluid communication with the vent opening via the equalization bypass when the valve member is at the first seal position.

15. The apparatus of claim 14, further comprising:
an expandable assembly having a first member, a second member and a third member, the first member coupled to the elastomeric member, the second member coupled between the first member and the third member, the third member coupled to the valve member, the expandable assembly configured to transition from a first configuration to a second configuration when the elastomeric member moves within the container body; and
the valve member moves from the second seal position to the third seal position when the expandable assembly transitions from the first configuration to the second configuration to release the pressurized gas from the gas chamber to the exterior volume.

16. The apparatus of claim 1, wherein:
the container body is movable within the housing from a first container position to a second container position in response to the force exerted by the pressurized gas;
the movable seal member is coupled to the container body and is positioned between the container body and an inner wall of the housing; and
the movement of the container body in response to the force exerted by the pressurized gas moves the movable seal member relative to the housing from the first seal position to the second seal position to isolate the gas chamber from the exterior volume.

17. The apparatus of claim 1, further comprising:
a delivery control mechanism coupled to the medicament container assembly, the delivery control mechanism including a flow restriction member configured to regulate flow of the pressurized gas into the container body that acts on the elastomeric member, wherein:
the medicament container assembly is configured to move from a first container position to a second container position in response to the force exerted by the pressurized gas, and
the flow restriction member is configured to limit movement of the elastomeric member prior to the medicament container assembly being placed in the second container position.

18. The apparatus of claim 17, wherein:
the gas chamber is a first gas chamber;
the flow restriction member is configured to permit pressurized gas to pass from the first gas chamber to a second gas chamber, the second gas chamber is in fluid contact with the elastomeric member;
a first portion of the pressurized gas in the first gas chamber has a first pressure magnitude;
a second portion of the pressurized gas in the second gas chamber has a second pressure magnitude; and
the first pressure magnitude is greater than the second pressure magnitude.

19. The apparatus of claim 1, further comprising a needle coupled to a distal end portion of the container body.
